(19) United States
(12) Patent Application Publication
(10) Pub. No.: US 2010/0235103 A1
(43) Pub. Date: Sep. 16, 2010

(54) Method and apparatus for evaluating prothrombotic conditions

(76) Inventors: Wallace E. Carroll, Santa Barbara, CA (US); R. David Jackson, Alexandria, IN (US)

Correspondence Address: Frank J. Bonini, Jr.;86 The Commons at Valley Forge East, 1288 Valley Forge Road, P.O. Box 750, Valley Forge, PA, 19482-0750, US

(21) Appl. No.: 11/906,325

(22) Filed: Oct. 1, 2007

Related U.S. Application Data
(63) Continuation-in-part of application No. 11/359,667, filed on Feb. 22, 2006, now Pat. No. 7,276,377.
Application No. 11/359,667 is a continuation-in-part of application No. 10/662,043, filed on Sep. 12, 2003, now abandoned.
Application No. 10/662,043 is a continuation of application No. 10/428,708, filed on May 2, 2003, now abandoned.
(60) Provisional application No. 60/679,423, filed on May 10, 2005.

(51) Int. Cl. G06F 19/00; G01N 33/48

(52) U.S. Cl. 702/19

(57) ABSTRACT

Methods and apparatus are disclosed for determining a prothombotic condition, including a condition of hypercoagulability. The determination is based on the clotting of a sample of blood or blood components which involves reacting the sample with a clotting agent and recording time and absorbance values. A slope determination is utilized to determine an indicator for a prothrombotic condition. The indicator according to embodiments, may be determined through the derivation of an angle in conjunction with the clotting analysis and slope.

### CROSS-REFERENCE TO RELATED APPLICATIONS

This application is a continuation-in-part of U.S. application Ser. No. 11/359,667, filed on Feb. 22, 2006, issued as U.S. Pat. No. 7,276,377 on Oct. 2, 2007, which is a continuation-in-part of U.S. application Ser. No. 10/662,043, filed on Sep. 12, 2003, which is a continuation of U.S. application Ser. No. 10/428,708 filed on May 2, 2003; the application also claims priority to U.S. Provisional application Ser. No. 60/679,423, filed on May 10, 2005, the disclosures of which are herein incorporated by reference.

### BACKGROUND OF THE INVENTION

1. Field of the Invention

This invention relates to analyzing blood for carrying out coagulation studies and other chemistry procedures, including determining the presence of prothrombotic abnormalities such as conditions of hypercoagulability, and monitoring oral anticoagulant therapy to take into account the platelet count in determining prothrombin times (PT), and a new Anticoagulant Therapy Factor (nATF).

2. Description of the Prior Art

Testing of blood and other body fluids is commonly done in hospitals, labs, clinics and other medical facilities. For example, to prevent excessive bleeding or deleterious blood clots, a patient may receive oral anticoagulant therapy before, during and after surgery. Oral anticoagulant therapy generally involves the use of oral anticoagulants—a class of drugs which inhibit blood clotting. To assure that the oral anticoagulant therapy is properly administered, strict monitoring is accomplished and is more fully described in various medical technical literature, such as the articles entitled “PTs, PR, ISIs and INRs: A Primer on Prothrombin Time Reporting Parts I and II” respectively published November, 1993 and December, 1993 issues of Clinical Hemostasis Review, and herein incorporated by reference.

These technical articles disclose anticoagulant therapy monitoring that takes into account three parameters which are: International Normalized Ratio (INR), International Sensitivity Index (ISI) and prothrombin time (PT), reported in seconds. The prothrombin time (PT) indicates the level of prothrombin and blood factors V, VII, and X in a plasma sample and is a measure of the coagulation response of a patient. Also affecting this response may be plasma coagulation inhibitors, such as, for example, protein C and protein S. Some individuals have deficiencies of protein C and protein S. The INR and ISI parameters are needed so as to take into account various differences in instrumentation, methodologies and in thromboplastins' (Tps) sensitivities, used in anticoagulant therapy. In general, thromboplastins (Tps) used in North America are derived from rabbit brain, those previously used in Great Britain from human brain, and those used in Europe from either rabbit brain or bovine brain. The INR and ISI parameters take into account all of these various factors, such as the differences in thromboplastins (Tps), to provide a standardized system for monitoring oral anticoagulant therapy to reduce serious problems related to prior, during and after surgery, such as excessive bleeding or the formation of blood clots.

The ISI itself according to the WHO 1999 guidelines, Publication no. 889-1999, have coefficients of variation ranging from 1.7% to 8.1%. Therefore, if the ISI is used exponentially to determine the INR of a patient, then the coefficients of variation for the INR's must be even greater than those for the ISI range.

As reported in Part I (Calibration of Thromboplastin Reagents and Principles of Prothrombin Time Report) of the above technical article of the Clinical Hemostasis Review, the determination of the INR and ISI parameters are quite involved, and as reported in Part II (Limitation of INR Reporting) of the above technical article of the Clinical Hemostasis Review, the error yielded by the INR and ISI parameters is quite high, such as about up to 10%. The complexity of the interrelationship between the International Normalized Ratio (INR), the International Sensitivity Index (ISI) and the patient's prothrombin time (PT) may be given by the below expression (A),

wherein the quantity

$$[ \frac{{Patient}^{′} s PT}{Mean of PT Normal Range} ] \qquad ( A )$$

is commonly referred to as prothrombin ratio (PR):

$$INR = {[ \frac{{Patient}^{′} s PT}{Mean of PT Normal Range} ]}^{ISI} \qquad ( B )$$

The possible error involved with the use of International Normalized Ratio (INR) is also discussed in the technical article entitled “Reliability and Clinical Impact of the Normalization of the Prothrombin Times in Oral Anticoagulant Control” of E. A. Loeliger et al., published in Thrombosis and Hemostasis 1985; 53: 148-154, and herein incorporated by reference. As can be seen in the above expression (B), ISI is an exponent of INR which leads to the possible error involved in the use of INR to be about 10% or possibly even more. A procedure related to the calibration of the ISI is described in a technical article entitled “Failure of the International Normalized Ratio to Generate Consistent Results within a Local Medical Community” of V. L. Ng et al., published in Am. J. Clin. Pathol. 1993; 99: 689-694, and herein incorporated by reference.

The unwanted INR deviations are further discussed in the technical article entitled “Minimum Lyophilized Plasma Requirement for ISI Calibration” of L. Poller et al. published in Am. J. Clin. Pathol. February 1998, Vol. 109, No. 2, 196-204, and herein incorporated by reference. As discussed in this article, the INR deviations became prominent when the number of abnormal samples being tested therein was reduced to fewer than 20 which leads to keeping the population of the samples to at least 20. The paper of L. Poller et al. also discusses the usage of 20 high lyophilized INR plasmas and 7 normal lyophilized plasmas to calibrate the INR. Further, in this article, a deviation of +/−10% from means was discussed as being an acceptable limit of INR deviation. Further still, this article discusses the evaluation techniques of taking into account the prothrombin ratio (PR) and the mean normal prothrombin time (MNPT), i.e., the geometric mean of normal plasma samples.

The discrepancies related to the use of the INR are further studied and described in the technical article of V. L. NG et al., entitled, “Highly Sensitive Thromboplastins Do Not Improve INR Precision,” published in Am. J. Clin. Pathol., 1998; 109, No. 3, 338-346 and herein incorporated by reference. In this article, the clinical significance of INR discordance is examined with the results being tabulated in Table 4 therein and which are analyzed to conclude that the level of discordance for paired values of individual specimens tested with different thromboplastins disadvantageously range from 17% to 29%.

U.S. Pat. No. 5,981,285 issued on Nov. 9, 1999 to Wallace E. Carroll et al., which discloses a “Method and Apparatus for Determining Anticoagulant Therapy Factors” provides an accurate method for taking into account varying prothrombin times (PT) caused by different sensitivities of various thromboplastin formed from rabbit brain, bovine brain or other sources used for anticoagulant therapy. This method does not suffer from the relatively high (10%) error sometimes occurring because of the use of the INR and ISI parameters with the exponents used in their determination.

The lack of existing methods to provide reliable results for physicians to utilize in treatment of patients has been discussed, including in a paper by Davis, Kent D., Danielson, Constance F. M., May, Lawrence S., and Han, Zi-Qin, “Use of Different Thromboplastin Reagents Causes Greater Variability in International Normalized Ratio Results Than Prolonged Room Temperature Storage of Specimens,” Archives of Pathol. and Lab. Medicine, November 1998. The authors observed that a change in the thromboplastin reagent can result in statistically and clinically significant differences in the INR.

Considering the current methods for determining anticoagulant therapy factors, there are numerous opportunities for error. For example, it has been reported that patient deaths have occurred at St. Agnes Hospital in Philadelphia, Pa. There the problem did not appear to be the thromboplastin reagent, but rather, was apparently due to a failure to enter the correct ISI in the instrument, used to carry out the prothrombin times when the reagent was changed. This resulted in the incorrect INR's being reported. Doses of coumadin were given to already overanticoagulated patients based on the faulty INR error, and it is apparent that patient deaths were caused by excessive bleeding due to coumadin overdoses.

But even in addition to errors where a value is not input correctly, the known methods for determining anticoagulant therapy factors still may be prone to errors, even when the procedure is carried out in accordance with the reagent manufacturer's ISI data. One can see this in that current methods have reported that reagents used to calculate prothrombin times, may, for healthy (i.e., presumed normal) subjects, give rise to results ranging from 9.7 to 12.3 seconds at the 95th % reference interval for a particular reagent, and 10.6 to 12.4 for another. The wide ranges for normal patients illustrates the mean normal prothrombin time differences. When the manufacturer reference data ranges are considered, if indeed 20 presumed normal patients' data may be reported within a broad range, then there is the potential for introduction of this range into the current anticoagulation therapy factor determinations, since they rely on the data for 20 presumed normal patients. Considering the reagent manufacturer expected ranges for expected normal prothrombin times, INR units may vary up to 30%. This error is apparently what physicians must work with when treating patients. A way to remove the potential for this type of error is needed.

This invention relates to the inventions disclosed in U.S. Pat. Nos. 3,905,769 ('769) of Sep. 16, 1975; 5,197,017 ('017) dated Mar. 23, 1993; and 5,502,651 ('651) dated Mar. 26, 1996, all issued to Wallace E. Carroll and R. David Jackson, and all of which are incorporated herein by reference. The present invention provides apparatus and methods for monitoring anticoagulant therapy and conditions relating to prothrombotic abnormalities, such as, for example, a hypercoagulation condition.

The blood and blood components of individual beings are often measured to evaluate levels of particular substances, including exogenous as well as endogenous molecules and compounds. Blood may be evaluated for blood abnormalities which relate to clotting (or the inability to clot). For example, blood and blood components may be measured in conjunction with blood clotting evaluations and analyses for determining treatment levels for the administration of anticoagulants, such as oral anticoagulant therapy, referred to above. For example, patients being treated or cared for, for certain heart or blood disorders, may receive blood thinning agents as a course of therapy. Some individuals exhibit what is clinically average or normal coagulation, whereas in others, the ability of their blood to coagulate may be referred to as a hypercoagulable condition, where clotting of the blood is considered to occur more rapidly than that of the clinically average individual. Conversely, another clinical condition is hypocoagulability, where the blood clotting requires additional time than that of the clinically average individual.

Hypercoagulability is a state of a person which involves an increased clotting function of the blood relative to what is considered to be presumed normal coagulation. Individuals presenting with hypercoagulable states have the potential to develop arterial or venous thromboembolism (VTE). Components considered to be responsible for effecting clot formation include fibrinogen, Factor VIII, von Willebrand Factor (VWF) and Factor XIII. Factor VIII is considered not to participate in the Prothrombin Time (time to the first clot formation). VWF concerns platelets. Platelets may be removed by centrifugation, such as where a plasma sample of the blood components is separated from the platelets. It is considered that thrombin is the component in blood responsible for the clotting to occur. The presence' of too much free thrombin is considered to be a condition hypercoagulability, and the lack or inactivity of thrombin results in the condition of hypocoagulability. Both, hypercoagulability and hypocoagulability, are conditions or states which may be brought about by various pathological conditions.

It is clinically important to know the state of an individual's clotting function, that is, in particular, where the individual is hypercoagulable, since treatments may be altered to account for this condition. In many cases, the presence of, or suspicion of, hypercoagulability is used to drive further treatments or testing of a patient, which may be very costly. Currently, the determination of hypercoagulability for an individual may take as long as about thirty minutes. See e.g., J. L. Curnow, et al., J. Thrombosis and Haemostasis, 5, 528-534 (2006). During the time it takes to make the determination, many things may happen, and, in many instances, the administration of treatment agents to an individual may be required prior to the time of completion of the hypercoagulability determination.

A prior method is the von Clauss fibrinogen method, which is based on the consideration that the greater the amount of fibrin present, the less the time for the thrombin clot time. However, the prior methods for determining hypercoagulability as a state of a person's blood, including the von Clauss method, have generally involved lengthy durations. Another example of a prior reported attempt to clinically determine hypercoagulable states is discussed in “Reduced fibrinolysis and increased fibrin generation can be detected in hypercoagulable patients using the overall haemostatic potential assay,” J. L. Curnow, et al., J. Thrombosis and Haemostasis, 5, 528-534 (2006). However, the Curnow determination proceeded over a course of minutes, where maximum optical density (OD) was not attained until after about 50 minutes, and where the first detection response appears to be after 5 minutes. (Id. at 530) Given the immediacy with which, in many situations, hypercoagulation must be resolved, or treatment option's for a patient considered, the time duration of thirty minutes, provided by prior methods, or even on the order of magnitude of minutes for prior determinations, may place many patients at a disadvantage or at an increased risk, including any of the risks associated with the condition of hypercoagulability. Often, further costly tests are given to patients who present with symptoms that may be clinically associated with hypercoagulable conditions. In some cases, these tests are unnecessary, adding further costs to patient care, and subjecting the patient to further waiting or discomfort. A need exists for a method and apparatus that may facilitate a determination of a hypercoagulable condition with speed and accuracy, and in an economical manner.

### SUMMARY OF THE INVENTION

Methods and apparatus useful for processing coagulation studies, and other chemistry procedures involving blood and blood components. The apparatus and methods may be used to determine anticoagulant therapy factors which are designated herein, in particular, to determine new Anticoagulant Therapy Factors (nATF's) which preferably may replace International Normalized Ratio (INR) in anticoagulation therapy management. Previously, anticoagulation therapy involved the use of International Normalized Ratios (INR's). The International Normalized Ratio (INR) was utilized in order to arrive at an anticoagulant therapy factor (ATF). The INR based ATF was dependent on the prothrombin time (PT), the prothrombin ratio (PR), a fibrinogen transformation rate (FTR), and a maximum acceleration point (MAP) having an associated time to maximum acceleration (TMA).

Methods and apparatus are disclosed for determining a new anticoagulant therapy factor (nATF) for monitoring oral anticoagulant therapy to help prevent excessive bleeding or deleterious blood clots that might otherwise occur before, during or after surgery. In one embodiment, a new anticoagulant therapy factor (nATF) is based upon a determination of the fibrinogen transformation rate (FTR) which, in turn, is dependent on a maximum acceleration point (MAP) for fibrinogen (FBG) conversion. The nATF quantity is also based upon the time to maximum acceleration from the time of reagent injection (TX) into a plasma sample, but does not require the difficulty of obtaining prior art International Normalized Ratio (INR) and International Sensitivity Index (ISI) parameters. The International Normalized Ratio (INR) was created to relate all species' clotting material to human clotting material, and nATF can replace INR in anticoagulant therapy management.

In accordance with other embodiments, methods and apparatus are provided for determining an anticoagulation therapy factor, which do not require the use of a mean normal prothrombin time (MNPT) and ISI data. In other words, the need to obtain and calculate the prothrombin time of 20 presumed normal patients, is not required to determine an anticoagulant therapy factor.

In accordance with the present invention, there is provided apparatus and methods for carrying out coagulation studies and other chemical procedures and analyses.

According to embodiments of the invention, there is provided a method for determining a hypercoagulability condition in an individual. The method may include monitoring a sample of an individual's blood and/or blood components for changes associated with fibrinogen to fibrin formation.

An apparatus for determining a hypercoagulable condition involving monitoring of a sample of an individual's blood and/or blood components for changes associated with fibrinogen to fibrin formation also is provided by the invention.

The method and apparatus may be used for determining the presence of a hypercoagulable state of a patient in an effective and efficient manner. According to preferred embodiments of the invention, the method and apparatus may facilitate making a determination of hypercoagulability within seconds.

Embodiments of the method and apparatus may include regulating further screening, testing and/or therapy programs by evaluating for a potential hypercoagulable state of a patient. A further object and advantage of the invention is to prevent the ordering of extensive laboratory tests. Preventing unnecessary testing has a benefit of convenience and comfort to a patient, as well as the economic value and benefit of costs savings to patients and healthcare insurers.

According to embodiments of the invention relating to the determination of a hypercoagulable condition, the method and apparatus further may include monitoring a voltage signal of a spectrophotometer to determine fibrinogen to fibrin formation in conjunction with or association with the readings taken of the sample to evaluate the passage and/or absorption of particular wavelengths or a spectral range.

According to preferred embodiments, the method and apparatus may be used to determine hypercoagulable conditions in an individual which are due to one or more or numerous conditions causing the condition. In other words, preferred embodiments may determine the presence of a hypercoagulable condition occurring from a different cause.

The methods and apparatus of the present invention are designed to provide an effective way to detect a hypercoagulability condition in a human, and within times of as short as about thirty seconds, as opposed to prior determinations which were on the order of thirty minutes.

### BRIEF DESCRIPTION OF THE DRAWINGS

FIG. 1 is a diagram of potentiophotometric apparatus constructed in accordance with one embodiment of the present invention for determining blood chemistry analyses such as coagulation studies, including determination of the new anticoagulant therapy factor (nATF), where the output of the analog/digital (A/D) converter is applied to a computer.

FIG. 2 is a plot of the various phases of the fibrinogen concentration occurring in a typical plasma clotting process.

FIG. 3 is another plot of the various phases of the fibrinogen concentration occurring in a typical plasma clotting process.

FIG. 4 is another plot of the various phases of the fibrinogen concentration occurring in a typical plasma clotting process.

FIG. 5 is another plot of the various phases of the fibrinogen concentration occurring in a typical plasma clotting process illustrating the fibrinogen lag phase.

FIG. 6 is another plot of the various phases of the fibrinogen concentration occurring in a typical plasma clotting process, illustrating an embodiment showing a representation of hypercoagulability data, including an example of an angle θ°.

FIGS. 7 and 8 represent graphs illustrating plots of the various phases of the fibrinogen concentration occurring in a plasma clotting process an analysis used to determine the presence of a hypercoagulable condition, wherein FIG. 7 relates to the presumed presence of a hypercoagulable condition and shows data corresponding to a high standard (HSTPC1). FIG. 8 illustrates a plot for a sample of an individual having coagulation which is presumed normal (corresponding to Sample ID 01 (for TPC).

### DETAILED DESCRIPTION OF THE INVENTION

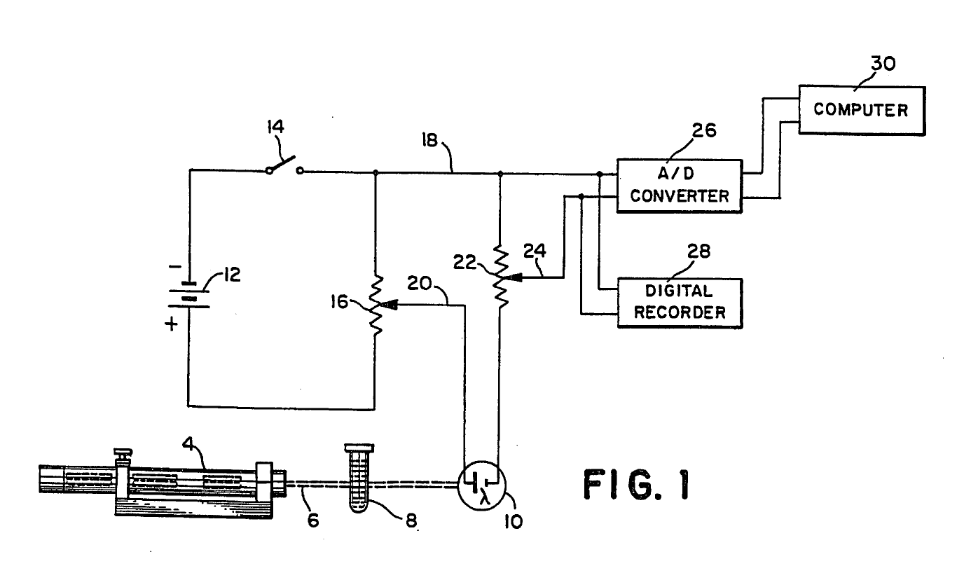

Referring to the drawings, wherein the same reference numbers indicate the same elements throughout, there is shown in FIG. 1 a light source 4 which may be a low power gas laser, or other light producing device, producing a beam of light 6 which passes through a sample test tube, such as the container 8, and is received by detection means which is preferably a silicon or selenium generating photocell 10 (photovoltaic cell). Battery 12 acts as a constant voltage DC source. Its negative terminal is connected through switch 14 to one end of variable resistor 16 and its positive terminal is connected directly to the opposite end of variable resistor 16. The combination of battery 12 and variable resistor 16 provides a variable DC voltage source, the variable voltage being derivable between line 18 at the upper terminal of resistor 16 and wiper 20. This variable DC voltage source is connected in series with detection means photocell 10, the positive output of detection means photocell 10 being connected to the wiper 20 of variable resistor 16 so that the voltage produced by the variable voltage DC source opposes the voltage produced by the detection means photocell 10. The negative output of detection means photocell 10 is connected through variable resistor 22 to line 18. Thus, the voltage across variable resistor 22 is the difference between the voltage produced by the variable voltage DC source and the voltage produced by the photovoltaic cell 10. The output of the electrical network is taken between line 18 and wiper 24 of variable resistor 22. Thus, variable resistor 22 acts as a multiplier, multiplying the voltage produced as a result of the aforesaid subtraction by a selective variable depending on the setting of variable resistor 22. The potentiophotometer just described embodies the electrical-analog solution to Beer's Law and its output is expressed directly in the concentration of the substance being measured.

Wiper 24 is illustrated placed at a position to give a suitable output and is not varied during the running of the test. The output between line 18 and wiper 24 is delivered to an A/D converter 26 and digital recorder 28. As is known, the A/D converter 26 and the digital recorder 28 may be combined into one piece of equipment and may, for example, be a device sold commercially by National Instrument of Austin, Tex. as their type Lab-PC+. The signal across variable resistor 22 is an analog signal and hence the portion of the signal between leads 18 and wiper 24, which is applied to the A/D converter 26 and digital recorder 28, is also analog. A computer 30 is connected to the output of the A/D converter 26, is preferably IBM compatible, and is programmed in a manner described hereinafter.

For example, preferably, the detector cell 10 is positioned adjacent an opposite wall of the sample container 8, and the emitter light source 4 positioned adjacent on opposite wall, so the light 6 emitted from the light source 4 passes through the container 8. The light source 4 is preferably selected to produce light 6 which can be absorbed by one or more components which are to be measured.

The apparatus can be used to Carry out coagulation studies in accordance with the invention. In accordance with a preferred embodiment of the present invention, the light source 4 may, for example, comprise a light emitting diode (LED) emitting a predetermined wavelength, such as for example, a wavelength of 660 nm, and the detector cell 10 may, for example, comprise a silicon photovoltaic cell detector. Optionally, though not shown, a bar code reader may also be provided to read bar code labels placed on the sample container 8. The bar code reader may produce a signal which can be read by the computer 30 to associate a set of data with a particular sample container 8.

To carry out a coagulation study on blood plasma, the citrated blood is separated from the red blood cell component of the blood. Conventional methods of separation, which include centrifugation, may be employed. Also, the use of a container device such as that disclosed in our issued U.S. Pat. No. 6,706,536, may also be used, and the method disclosed therein for reading the plasma volume relative to the sample volume may also be employed.

Illustrative of an apparatus and method according to one embodiment is a coagulation study which can be carried out therewith. A reagent, such as, for example, Thromboplastin-Calcium (Tp-Ca), is added to the plasma sample which is maintained at about 37° C. by any suitable temperature control device, such as a heated sleeve or compartment (not shown). The reagent addition is done by dispensing an appropriate amount of the reagent into the plasma portion of the blood. The plasma portion may be obtained by any suitable separation technique, such as for example, centrifugation. In one embodiment illustrated herein, the container 8 is vented when reagent is added. The reagent for example, may comprise thromboplastin, which is added in an amount equal to twice the volume of the plasma. The reagent is mixed with the plasma. It is preferable to minimize air bubbles so as not to interfere with the results. The plasma sample to which the reagent has been added is heated to maintain a 37° C. temperature, which, for example, may be done by placing the container holding the plasma and reagent in a heating chamber (not shown).

Readings are taken of the optical activity of the components in the sample container 8.

Reaction kinematics may be studied by observing changes in the optical density of the plasma layer. For example, an amount of reagent, such as Thromboplastin-Calcium (Tp-Ca), may be added to the plasma sample in the container. The plasma sample in the container may comprise a known amount of volume. Alternately, the plasma volume may be ascertained through the method and apparatus described in our U.S. Pat. No. 6,706,536. A controlled amount of Tp-Ca reagent is added to the plasma sample. The amount of reagent added corresponds to the amount of plasma volume. The detector cell 10 and emitter light source 4 are preferably positioned so the absorbance of the plasma sample may be read, including when the reagent is added and the sample volume is thereby increased.

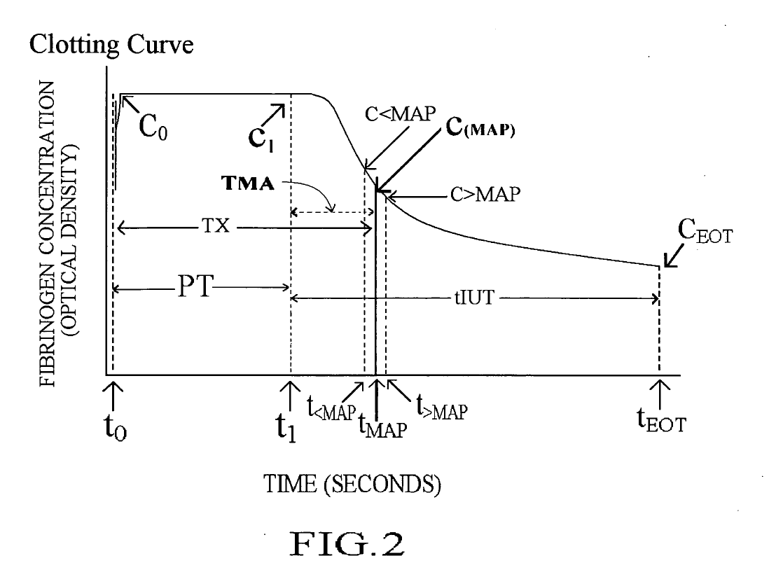

With the detection elements, such as the cell 10 and emitter 4, positioned to read the plasma sample and the reagents added thereto, the reaction analysis of the extended prothrombin time curve can be followed. FIG. 2 shows a graph of a plot of the various phases of the fibrinogen concentration occurring in a typical plasma clotting process. The change in optical density of the plasma level occurs after reagents have been added. The optical density of the plasma sample is monitored, as optically clear fibrinogen converts to turbid fibrin.

The coagulation study of the type described above is used to ascertain the results shown in the graph plotted on FIG. 2. The description of the analysis makes reference to terms, and symbols thereof, having a general description as used herein, all to be further described and all of which are given in Table 1.

TABLE 1

| SYMBOL | TERM | GENERAL DESCRIPTION |
|---|---|---|
| PT | Prothrombin Time | A period of time calculated from the addition of the |
| | | reagent (e.g., thromboplastin-calcium) to a point where |
| | | the conversion of fibrinogen to fibrin begins (i.e. the |
| | | formation of the first clot). |
| TMA | Time to Maximum | The time from PT to a point where the rate of conversion |
| | Acceleration | of fibrinogen to fibrin has reached maximum and begins |
| | | to slow. |
| MAP | Maximum Acceleration Point | A point where the fibrinogen conversion achieves |
| | | maximum acceleration and begins to decelerate. |
| EOT | End of Test | Point where there is no appreciable change in the |
| | | polymerization of fibrin. |
| TEOT | Theoretical End Of Test | The time to convert all fibrinogen based on the time to |
| | | convert the fibrinogen during the simulated Zero Order |
| | | Kinetic rate. |
| TX (or T₂) | Time to Map | Time to reach the Maximum Acceleration Point (MAP) |
| | | from point of injection. |
| MNTX | Mean Normal Time to Map | The mean of the times of at least 20 normal people to |
| | | reach then Maximum Acceleration Point (MAP). |
| FTR | Fibrinogen Transformation | The amount of fibrinogen converted during a particular |
| | Ratio | time period. This is a percentage of the total Fibrinogen. |
| ATF | Anticoagulation Therapy | The calculated value used to monitor the uses of an |
| | Factor | anticoagulant without a need for an International |
| | | Sensitivity Index (ISI) of a thromboplastin. |
| nATF | new Anticoagulation Therapy | A replacement for the INR to provide a standardized |
| | Factor | system for monitoring oral anticoagulant therapy. (Also |
| | | expressed as ATFt and ATFz) |
| PR | Prothrombin Ratio | A value computed by dividing a sample PT by the |
| | | geometric mean of at least 20 normal people (MNPT). |
| INR | International Normalized | A parameter which takes into account the various factors |
| | Ratio | involved in anticoagulation therapy monitoring to provide |
| | | a standardized system for monitoring oral anticoagulant |
| | | therapy. |
| ATFt | Anticoagulation Therapy | Utilizing a calculated Theoretical End Of Test value and |
| | Factor Theoretical | the Natural Log “e” to removed the need for an MNPT. |
| XR | Time to MAP Ratio | The value computed by dividing a sample “TX” by the |
| | | geometric mean of at least 20 normal people “MNTX”. |

Prior patents for obtaining an anticoagulant therapy factor (ATF) relied on the International Normalized Ratio (INR) system which was derived in order to improve the consistency of results from one laboratory to another. The INR system utilized the calculation of INR from the equation:

INR=(PTₚₐₜᵢₑₙₜ/PT_{geometric mean})^{ISI}

wherein the PTₚₐₜᵢₑₙₜ is the prothrombin time (PT) as an absolute value in seconds for a patient, PT_{geometric mean} is the mean, a presumed number of normal patients. The International Sensitivity Index (ISI) is an equalizing number which a reagent manufacturer of thromboplastin specifies. The ISI is a value which is obtained through calibration against a World Health Organization primary reference thromboplastin standard. Local ISI (LSI) values have also been used to provide a further refinement of the manufacturer-assigned ISI of the referenced thromboplastin in order to provide local calibration of the ISI value.

For illustration, the present invention can be employed for accurate determination of a new Anticoagulant Therapy Factor (nATF) from a human blood sample, for use during the monitoring of oral anticoagulant therapy, without the need for an ISI or LSI value, and without the need for an INR value. As is known in the art, blood clotting Factors I, II, V, VII, VIII, IX and X are associated with platelets (Bounameaux, 1957); and, among these, Factors II, VII, IX and X are less firmly attached, since they are readily removed from the platelets by washing (Betterle, Fabris et al, 1977). The role of these platelet-involved clotting factors in blood coagulation is not, however, defined. The present invention provides a method and apparatus for a new Anticoagulant Therapy Factor (nATF) which may be used for anticoagulant therapy monitoring without the need for INR.

The International Normalized Ratio (INR) is previously discussed in already incorporated reference technical articles entitled “PTs, PRs, ISIs and INRs: A Primer on Prothrombin Time Reporting Part I and II respectively,” published in November, 1993 and December, 1993 issues of Clinical Hemostasis Review. The illustrative example of an analysis which is carried out employing the present invention relies upon the maximum acceleration point (MAP) at which fibrinogen conversion achieves a maximum and from there decelerates, the time to reach the MAP (TX), and the mean normal time to MAP (MNTX), and a fibrinogen transformation rate (FTR), that is, the thrombin activity in which fibrinogen (FBG) is converted to fibrin to cause clotting in blood plasma.

More particularly, during the clotting steps used to determine the clotting process of a plasma specimen of a patient under observation, a thromboplastin (Tp) activates factor VII which, activates factor X, which, in turn, under catalytic action of factor V, activates factor II (sometimes referred to as prothrombin) to cause factor IIa (sometimes referred to as thrombin) that converts fibrinogen (FBG) to fibrin with resultant turbidity activity which is measured, in a manner as to be described hereinafter, when the reaction is undergoing simulated zero-order kinetics.

From the above, it should be noted that the thromboplastin (Tp) does not take part in the reaction where factor IIa (thrombin) converts fibrinogen (FBG) to fibrin which is deterministic of the clotting of the plasma of the patient under consideration. The thromboplastin (Tp) only acts to activate factor VII to start the whole cascade rolling. Note also that differing thromboplastins (Tps) have differing rates of effect on factor VII, so the rates of enzyme factor reactions up to II-IIa (the PT) will vary.

Therefore, the prothrombin times (PTs) vary with the different thromboplastins (Tps) which may have been a factor that mislead authorities to the need of taking into account the International Normalized Ratio (INR) and the International Sensitivity Index (ISI) to compensate for the use of different types of thromboplastins (Tps) during the monitoring of oral anticoagulant therapy. It is further noted, that thromboplastins (Tps) have nothing directly to do with factor IIa converting fibrinogen (FBG) to fibrin, so it does not matter which thromboplastin is used when the fibrinogen transformation is a primary factor.

The thromboplastin (Tp) is needed therefore only to start the reactions that give factor IIa. Once the factor IIa is obtained, fibrinogen (FBG) to fibrin conversion goes on its own independent of the thromboplastin (Tp) used.

In one embodiment, the present method and apparatus has use, for example, in coagulation studies where fibrinogen (FBG) standard solutions and a control solution are employed, wherein the fibrinogen standard solutions act as dormant references to which solutions analyzed with the present invention are compared, whereas the control solution acts as a reagent that is used to control a reaction. The fibrinogen standards include both high and low solutions, whereas the control solution is particularly used to control clotting times and fibrinogens of blood samples. It is only necessary to use fibrinogen standards when PT-derived fibrinogens (FBG's) are determined. In connection with other embodiments of the invention, fibrinogen (FBG) standards are not necessary for the INR determination (such as for example INRz described herein).

Another embodiment provides a method and apparatus for determining an anticoagulation therapy factor which does not require the use of fibrinogen standard solutions. In this embodiment, the apparatus and method may be carried out without the need to ascertain the mean normal prothrombin time (MNPT) of 20 presumed normal patients.

Where a fibrinogen standard solution is utilized, a fibrinogen (FBG) solution of about 10 g/l may be prepared from a cryoprecipitate. The cryoprecipitate may be prepared by freezing plasma, letting the plasma thaw in a refrigerator and then, as known in the art, expressing off the plasma so as to leave behind the residue cryoprecipitate. The gathered cryoprecipitate should contain a substantial amount of both desired fibrinogen (FBG) and factor VIII (antihemophilic globulin), along with other elements that are not of particular concern to the present invention. The 10 g/l fibrinogen (FBG) solution, after further treatment, serves as the source for the high fibrinogen (FBG) standard. A 0.5 g/l fibrinogen (FBG) solution may then be prepared by a 1:20 (10 g/l/20=0.5 g/l) dilution of some of the gathered cryoprecipitate to which may be added an Owren's Veronal Buffer (pH 7.35) (known in the art) or normal saline solution and which, after further treatment, may serve as a source of the low fibrinogen (FBG) standard.

The fibrinogen standard can be created by adding fibrinogen to normal plasma in an empty container. Preferably, the fibrinogen standard is formed from a 1:1 fibrinogen to normal plasma solution. For example, 0.5 ml of fibrinogen and 0.5 ml of plasma can be added together in an empty container. Thromboplastin calcium is then added to the fibrinogen standard. Preferably, twice the amount by volume of thromboplastin is added into the container per volume amount of fibrinogen standard which is present in the container. The reaction is watched with the apparatus 10.

Then, 1 ml of each of the high (10 g/l) and low (0.5 g/l) sources of the fibrinogen standards may be added to 1 ml of normal human plasma (so the cryoprecipitate plasma solution can clot). Through analysis, high and low fibrinogen (FBG) standards are obtained. Preferably, a chemical method to determine fibrinogen (FBG) is used, such as, the Ware method to clot, collect and wash the fibrin clot and the Ratnoff method to dissolve the clot and measure the fibrinogen (FBG) by its tyrosine content. The Ware method is used to obtain the clot and generally involves collecting blood using citrate, oxalate or disodium ethylenediaminetetraacetate as anticoagulant, typically adding 1.0 ml to about 30 ml 0.85% or 0.90% sodium chloride (NaCl) in a flask containing 1 ml M/5 phosphate buffer and 0.5 ml 1% calcium chloride CaCl₂, and then adding 0.2 ml (100 units) of a thrombin solution. Preferably, the solution is mixed and allowed to stand at room temperature for fifteen minutes, the fibrin forming in less than one minute forming a solid gel if the fibrinogen concentration is normal. A glass rod may be introduced into the solution and the clot wound around the rod. See Richard J. Henry, M.D., et al., Clinical Chemistry: Principals and Techniques (2^{nd} Edition) 1974, Harper and Row, pp. 458-459, the disclosure of which is incorporated herein by reference. Once the clot is obtained, preferably the Ratnoff method may be utilized to dissolve the clot and measure the fibrinogen (FBG) by its tyrosine content. See “A New Method for the Determination of Fibrinogen in Small Samples of Plasma”, Oscar D. Ratnoff, M. D. et al., J. Lab. Clin. Med., 1951: V. 37 pp. 316-320, the complete disclosure of which is incorporated herein by reference. The Ratnoff method relies on the optical density of the developed color being proportional to the concentration of fibrinogen or tyrosine and sets forth a calibration curve for determining the relationship between optical density and concentration of fibrinogen. The addition of a fibrinogen standard preferably is added to the plasma sample based on the volume of the plasma.

As is known, the addition of the reagent Thromboplastin C serves as a coagulant to cause clotting to occur within a sample of citrated blood under test which may be contained in a container 8. As clotting occurs, the A/D converter 26 of FIG. 1 will count and produce a digital value of voltage at a predetermined period, such as once every 0.05 or 0.01 seconds. As more fully described in the previously incorporated by reference U.S. Pat. No. 5,197,017 ('017), these voltage values are stored and then printed by the recorder as an array of numbers, the printing being from left to right and line by line, top to bottom. There are typically one hundred numbers in the five groups representing voltage values every second and hence, one line represents one-fifth of a second in time (20×0.01 seconds). Individual numbers in the same column are twenty sequential numbers apart. Hence, the time difference between two adjacent numbers in a column is one-fifth of a second. The significance of these recorded values may be more readily appreciated after a general review of the operating principles illustrated in FIG. 2 having a Y axis identified as Fibrinogen Concentration (Optical Density) and an X axis identified in time (seconds).

FIG. 2 illustrates the data point locations of a clotting curve related to a coagulation study which illustrates the activation and conversion of fibrinogen to fibrin. In general, FIG. 2 illustrates a “clot slope” method that may be used in a blood coagulation study carried out for determining a new anticoagulant therapy factor (nATFa). The ATFa represents an anticoagulation therapy factor represented by the expression ATFa=XR^{(2-nFTR)} wherein a maximum acceleration point is obtained, and nFTR=IUX/IUT, where IUX is the change in optical density from a time prior to the MAP time (t_{<MAP} which is t_{MAP} minus some time from MAP) to the optical density at a time after the MAP time (t_{>MAP} which is t_{MAP} plus some time from MAP); and wherein IUT=the change in optical density at the time t₁ to the optical density measured at time t_{EOT}, where time t_{EOT} is the end of the test (EOT). The first delta (IUX) represents the fibrinogen (FBG) for MAP (−a number of seconds) to MAP (+a number of seconds) (that is the fibrinogen (FBG) converted from t_{<MAP} to t_{>MAP} on FIG. 2) The (IUT) represents fibrinogen converted from c₁ to c_{EOT} (that is the fibrinogen converted from t₁ to t_{EOT}, see FIG. 2). The XR for the ATFa expression is XR=TX/MNTX, which is the ratio of time to map (TX) by the mean normal time to map of 20 presumed “normal” patients.

The study which measures the concentration of the fibrinogen (FBG) in the plasma that contributes to the clotting of the plasma and uses an instrument, such as, for example, the potentiophotometer apparatus illustrated in FIG. 1, to provide an output voltage signal that is directly indicative of the fibrinogen (FBG) concentration in the plasma sample under test, is more fully discussed in the previously incorporated by reference U.S. Pat. No. 5,502,651. The quantities given along the Y-axis of FIG. 2 are values (+ and −) that may be displayed by the digital recorder 28. The “clot slope” method comprises detection of the rate or the slope of the curve associated with the formation of fibrin from fibrinogen. The “clot slope” method takes into account the time to maximum acceleration (TX) which is the point at which fibrinogen conversion achieves a maximum and from there decelerates.

As seen in FIG. 2, at time t₀, corresponding to a concentration c₀, the thromboplastin/calcium ion reagent is introduced into the blood plasma which causes a disturbance to the composition of the plasma sample which, in turn, causes the optical density of the plasma sample to increase momentarily. After the injection of the reagent (the time of which is known, as to be described, by the computer 30), the digital quantity of the recorder 28 of FIG. 1 rapidly increases and then levels off in a relatively smooth manner and then continues along until the quantity c₁ is reached at a time t₁. The time which elapses between the injection of thromboplastin at t₀ and the instant time t₁ of the quantity c₁ is the prothrombin time (PT) and is indicated in FIG. 2 by the symbol PT. As shown in FIG. 2, the baseline that develops after the thromboplastin (TP) is introduced or injected into the sample generally is thought to represent the “lag phase” of all of the enzymes preceding prothrombin converting to fibrin. The enzymes types and amounts may vary from person to person, and thus, this would demonstrate the potential for prothrombin times to vary between individuals.

An anticoagulant therapy factor (nATF) is determined. The optical density of a quantity c₁ directly corresponds to a specified minimum amount of fibrinogen (FBG) that must be present for a measuring system, such as the circuit arrangement of FIG. 1, to detect in the plasma sample that a clot is being formed, i.e., through the transformation of fibrinogen to fibrin. The quantities shown in FIG. 2 are of optical densities, which may be measured in instrument units, that are directly correlatable to fibrinogen concentration values. The quantity c₁, may vary from one clot detection system to another, but for the potentiophotometer system of FIG. 1, this minimum is defined by units of mass having a value of about 0.05 grams/liter (g/l).

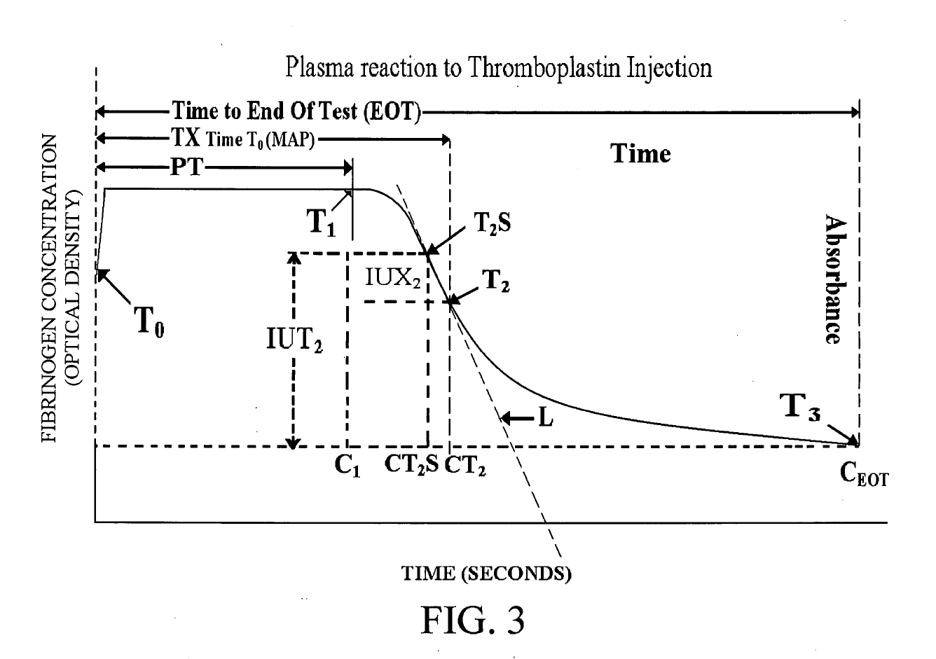

Considering the clotting curve of FIG. 2, detection of a first predetermined quantity c₁ is illustrated occurring at a corresponding time t₁, which is the start of the clotting process. In accordance with one or more embodiments, this process may be monitored with the apparatus of FIG. 1 for determining a new anticoagulant therapy factor (nATF). The time t₁ is the beginning point of the fibrinogen formation, that is, it is the point that corresponds to the beginning of the acceleration of the fibrinogen conversion that lasts for a predetermined time, The acceleration of the fibrinogen conversion proceeds from time (t₁) and continues until a time t_{MAP}, having a corresponding quantity c_{MAP}. The time t_{MAP}, as well as the quantity c_{MAP}, is of primary importance because it is the point of maximum acceleration of the fibrinogen (FBG) to fibrin conversion and is also the point where deceleration of fibrinogen (FBG) to fibrin conversion begins. Further, the elapsed time from t₀ to t_{MAP} is a time to maximum acceleration from reagent injection (TX), shown in FIG. 2. Preferably, the conversion of fibrinogen to fibrin is quantified every 0.1 seconds. The time to maximum acceleration from reagent injection (TX) is defined as, the point on the clotting curve time line where this conversion has reached its maximum value for the last time, simulating a zero-order kinetic rate. To facilitate ascertainment of the location point of the last maximum value, the delta value of two points at a fixed interval may be measured until this value begins to decrease. This value is tracked for a period of time, such as for example five seconds, after the first decreasing value has been determined. This facilitates ascertainment of the last point of what may be referred to as a simulated zero-order kinetic rate. Referring to FIG. 3, a zero order kinetic rate is illustrated by the line (L).

As shown in FIG. 2, a quantity c_{MAP} and a corresponding time t_{MAP} define a maximum acceleration point (MAP). Fibrin formation, after a short lag phase before the MAP, occurs for a period of time, in a linear manner. Fibrinogen (FBG) is in excess during this lag phase, and fibrin formation appears linear up to the MAP.

The deceleration of fibrinogen (FBG) to fibrin conversion continues until a quantity c_{EOT} is reached at a time t_{EOT}. The time t_{EOT} is the point where the deceleration of the fibrinogen (FBG) to fibrin conversion corresponds to a value which is less than the required amount of fibrinogen (FBG) that was present in order to start the fibrinogen (FBG) to fibrin conversion process. Thus, because the desired fibrinogen (FBG) to fibrin conversion is no longer in existence, the time t_{EOT} represents the ending point of the fibrinogen (FBG) to fibrin conversion in accordance with the coagulation study exemplified herein, which may be referred to as the end of the test (EOT). The fibrinogen (FBG) to fibrin conversion has a starting point of t₁ and an ending point of t_{EOT}. The differential of these times, t₁ and t_{EOT}, define a second delta (IUT).

The “clot slope” method that gathers typical data as shown in FIG. 2 has four critical parameters. The first is that the initial delta optical density of substance being analyzed should be greater than about 0.05 g/l in order for the circuit arrangement of FIG. 1 to operate effectively. Second, the acceleration fibrinogen (FBG) to fibrin conversion should be increasing for a minimum period of about 1.5 seconds so as to overcome any false reactions created by bubbles. Third, the total delta optical density (defined by the difference in quantities c₁ and c_{EOT}) should be at least three (3) times the instrument value in order to perform a valid test, i.e., (3)*(0.05 g/l)=0.15 g/l. Fourth, the fibrinogen (FBG) to fibrin conversion is defined, in part, by the point (t_{EOT}) where the deceleration of conversion becomes less than the instrument value of about 0.05 g/l that is used to detect the clot point (t₁). As with most clot detection systems, a specific amount of fibrinogen needs to be present in order to detect a clot forming. Adhering to the four given critical parameters is an example of how the present apparatus and method may be used to carry out a coagulation study to determine a specific quantity of fibrinogen. In order for that specific amount of fibrinogen to be determined, it is first necessary to detect a clot point (t₁). After that clot point (t₁) is detected, it logically follows that when the fibrinogen conversion becomes less than the specific amount (about 0.05 g/l for the circuit arrangement of FIG. 1), the end point (t_{EOT}) of the fibrinogen conversion has been reached.

One embodiment of the method and apparatus is illustrated in accordance with the clotting curve shown in FIG. 3. The clotting curve of FIG. 3 illustrates the values ascertained in arriving at a new anticoagulation therapy factor (nATFz). The embodiment illustrates the determination of a new anticoagulation therapy factor (nATFz), expressed by the following formula:

nATFz=XR^{(2-nFTR)} (1)

This embodiment utilizes a zero order line (L) to obtain a first delta, in particular IUXz, which is a first differential taken along the simulated zero order kinetic line (L), and preferably along the segment between the start of the simulated zero order kinetic (T₂S) to the last highest absorbance value (T₂) (i.e., preferably, the last highest absorbance value of a simulated zero order kinetic). As previously discussed, the acceleration of the fibrinogen conversion proceeds from a first time, here time (T₁) and continues, eventually reaching a time where the last highest delta absorbance value or maximum acceleration point (T₂) having a corresponding quantity c_{T2} is reached. The values for “T” correspond with times, and the values for “c” correspond with quantity, which may be measured in instrument units based on optical density readings (also referred to as optical density or o.d.). The time T₂, as well as the quantity c_{T2}, is the point of maximum acceleration of the fibrinogen (FBG) to fibrin conversion and is also the point where deceleration of fibrinogen (FBG) to fibrin conversion begins. In this embodiment, IUXz is the change in optical density preferably from the beginning of the at the time T₂S at which the simulated zero order kinetic begins to the optical density at time T₂ which is the maximum acceleration point or the last highest delta absorbance value of a simulated zero order kinetic. FIG. 3 shows the differential IUXz taken between a preferred segment of the zero order line. The second delta in particular (IUTz) is the change in optical density at the time T₂S to the optical density measured at time T₃, where time T₃ is the end of the test (EOT).

The (IUXz) represents the fibrinogen (FBG) converted between time T₂S and T₂. The (IUTz) represents fibrinogen converted from the time T₂S to the end of the test or T₃.

The maximum acceleration ratio (XR) for this embodiment is calculated to arrive at the new alternate anticoagulation therapy factor (nATFz). The maximum acceleration ratio (XR) is defined as the time to maximum acceleration from reagent injection (TX) divided by the mean normal TX value of a number of presumed normal specimens (MNTX). For example, the mean normal TX value may be derived based on the value of 20 or more presumed normal specimens. The maximum acceleration ratio (XR) may be expressed through the following formula:

XR=TX/MNTX (2)

The clotting curve of FIG. 3 illustrates the values ascertained in arriving at the new alternate anticoagulation therapy factor (nATFz). The new alternate anticoagulation therapy factor (nATFz) is preferably expressed by the following formula:

nATFz=XR^{(2-nFTR)} (3)

with FTR being IUXz/IUTz.

The preferred IBM-compatible computer 30 of FIG. 1 stores and manipulates these digital values corresponding to related data of FIG. 3 and is preferably programmed as follows:
(a) a sample of blood where the plasma is available, such as, for example, a sample of citrated blood, is obtained and placed in an appropriate container, the computer 30, as well as the recorder 28, sequentially records voltage values for a few seconds before injection of thromboplastin. As previously discussed, thromboplastin (tissue factor) is one of the factors in the human body that causes blood to clot. Prothrombin is another. Fibrinogen is yet another. Before injection of the thromboplastin, the output from the A/D converter 26 is relatively constant. When thromboplastin is injected into the plasma sample in the container, a significant and abrupt change occurs in the recorded voltage values of both the computer 30 and the recorder 28. This abrupt change is recognized by both the recorder 28 and, more importantly, by the computer 30 which uses such recognition to establish T₀. The computer 30 may be programmed so as to correlate the digital quantities of the A/D converter 26 to the analog output of the detector means photocell 10 which, in turn, is directly correlatable to the fibrinogen (FBG) concentration g/l of the sample of blood discussed with reference to FIG. 3;

(b) the computer 30 may be programmed to look for a digital quantity representative of a critical quantity c₁, and when such occurs, record its instant time T₁. (The time span between Tₒ and T₁ is the prothrombin time (PT), and has an normal duration of about 12 seconds, but may be greater than 30 seconds);
(c) following the detection of the quantity c₁, the computer 30 may be programmed to detect for the acceleration of fibrinogen (FBG) to fibrin conversion. The computer 30 is programmed to detect the maximum acceleration quantity c_{MAP} or C_{T2} as illustrated in FIG. 3, and its corresponding time of occurrence t_{MAP}, which is T₂ in FIG. 3.
(d) the computer detects a quantity c_{EOT} occurring at time t_{EOT}. Typically, it is important that the rate of fibrin formation increase for at least 1.5 seconds following the occurrence of (T₁);
(e) The computer 30 is programmed to ascertain the value for the time to start (T₂S) which corresponds with the time at which the simulated zero order kinetic rate begins.
(f) following the detection of the acceleration of fibrinogen conversion to detect the start time T₂S, the computer 30 is programmed to detect for a deceleration of the fibrinogen conversion, wherein the fibrinogen concentration decreases from a predetermined quantity c_{MAP} to a predetermined quantity c_{EOT} having a value which is about equal but less than the first quantity c₁. The computer is programmed to ascertain a first delta (IUTz), by determining the difference between the quantity C_{T2S} and the quantity c_{EOT}; and a second delta (IUXz) by determining the difference between the quantity c_{T2S} and the quantity c₂ (or c_{MAP}).
(g) the computer 30 manipulates the collected data of (a); (b); (c); (d); (e) and (f) above, to determine the new fibrinogen transfer rate (nFTR). The nFTR may be arrived at based on the principle that if a required amount (e.g., 0.05 g/l) of fibrinogen concentration c₁ is first necessary to detect a clot point (T₁); then when the fibrinogen concentration (c_{EOT}) becomes less than the required amount c₁, which occurs at time (T_{EOT}), the fibrinogen end point has been reached. More particularly, the required fibrinogen concentration c₁ is the starting point of fibrinogen conversion of the clotting process and the less than required fibrinogen concentration c_{EOT} is the end point of the fibrinogen conversion of the clotting process.
(h) The computer now has the information needed to determine the new fibrinogen transfer rate (nFTRz) which is expressed by the following formula:

nFTRz=IUXz/IUTz (4)
(i) data collected is manipulated by the computer 30 to calculate the maximum acceleration ratio (XR), which is expressed as TX divided by the mean normal TX value of at least 20 presumed normal specimens (MNTX):

XR=TX/MNTX (2)

The MNTX value may be ascertained and stored in the computer for reference.
(j) the computer 30 now has the information needed to determine the nATFz, (also referred to as INRz) which typically is expressed as:

nATFz or INRz=XR^{(2-nFTR)} (3)

where, in the exponent, the value 2 is the logarithm of the total fibrinogen, which, as expressed in terms of the optical density, is 100% transmittance, the log of 100 being 2.

The new anticoagulation therapy factor (nATFz) does not require an ISI value, as was previously used to determine anticoagulation therapy factors. The new anticoagulation therapy factor (nATFz) uses for its ascertainment the values extracted from the clotting curve (see FIG. 3), in particular (nFTRz) (determined based on IUXz and IUTz), and (TX). In carrying out coagulation studies, the new anticoagulant therapy factor (nATFz) may replace INR in anticoagulant therapy management.

The apparatus and method for obtaining a new anticoagulant therapy factor, (nATFz), may be accomplished without encountering the complications involved with obtaining the prior art quantities International Normalized Ratio (INR) and International Sensitivity Index (ISI).

The new anticoagulant therapy factor (nATFz or ATF) preferably is a replacement for the International Normalized Ratio (INR), hence it may be referred to as INRz. Existing medical literature, instrumentation, and methodologies are closely linked to the International Normalized Ratio (INR). The nATFz was compared for correlation with the INR by comparative testing, to INR quantities, even with the understanding that the INR determination may have an error of about ten (10) % which needs to be taken into account to explain certain inconsistencies.

Table 2, below, includes anticoagulant therapy factors obtained from patients at two different hospitals. The ATFz values were obtained, with GATFz representing one geographic location where patients were located and MATFz being another location. The ATFz was obtained as the new anticoagulant therapy factor, and as illustrated in Tables 4 and 5, below, compares favorably to results obtained for INR determinations.

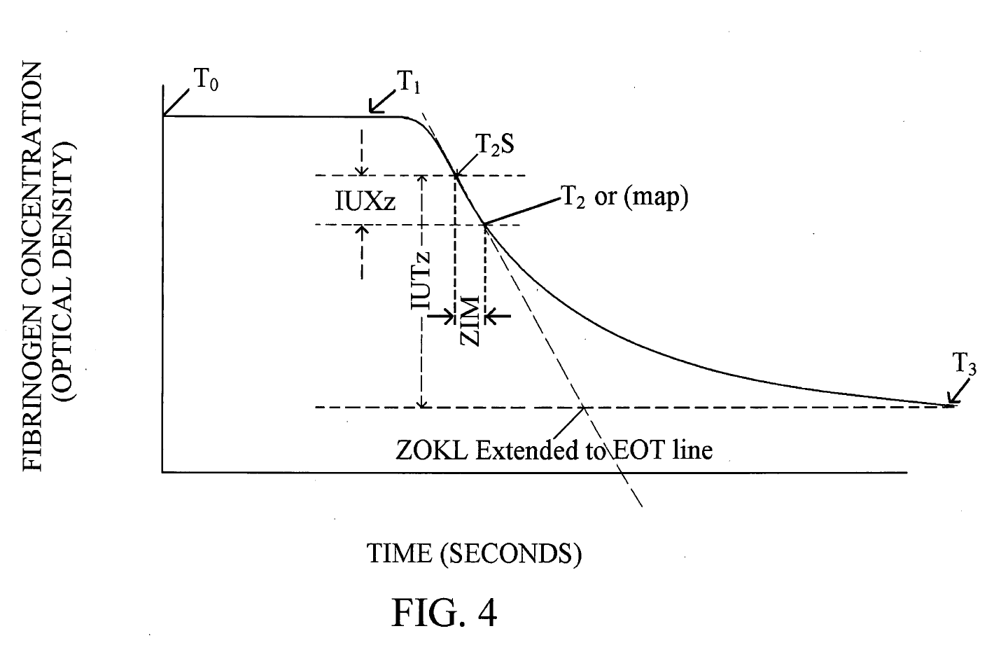

Another alternate embodiment for determining a new anticoagulant therapy factor (ATFt) is provided. The alternate embodiment for determining ATFt eliminates the need for determining a mean normal prothrombin time (MNPT) (or MNXT) and ISI, saving considerable time and costs, and removing potential sources of error, as the MNPT (the expected value of MNPT's depending on the varying 20 presumed normals population) and ISI (generally provided by the manufacturer of the reagent—such as, for example, the thromboplastin, etc.) are not required for the determination of the ATFt. An alternate embodiment for determining ATFt is illustrated in accordance with the clotting curve shown in FIG. 4. The clotting curve of FIG. 4 illustrates values ascertained in arriving at the alternate new anticoagulation therapy factor (nATFt). The alternate new anticoagulation therapy factor (nATFt) is preferably expressed by the following formula:

nATFt=Value 1*Value 2 (4)

The alternate embodiment utilizes the zero order line (L) to obtain a first delta, in particular IUXz, which is a first differential taken along the simulated zero order kinetic line (L), and preferably along the segment between the start of the simulated zero order kinetic (T₂S) to the last highest absorbance value (T₂) (i.e., preferably, the last highest absorbance value of a simulated zero order kinetic). As previously discussed, the acceleration of the fibrinogen conversion proceeds from a first time, here time (T₁) and continues, eventually reaching a time where the last highest delta absorbance value or maximum acceleration point (T₂) having a corresponding quantity c_{T2} is reached. The time T₂, as well as the quantity c_{T2}, is the point of maximum acceleration of the fibrinogen (FBG) to fibrin conversion and also is the point where deceleration of fibrinogen (FBG) to fibrin conversion begins. As illustrated on the clotting chart in FIG. 4, IUXz represents a change in optical density (o.d.) preferably from the beginning of the at the time T₂S at which the simulated zero order kinetic begins to the optical density at time T₂ which is the maximum acceleration point or the last highest delta absorbance value of a simulated zero order kinetic. The value IUXz is generally expressed in instrument units (corresponding to absorbance or percent transmittance) and may generally be referred to as optical density or o.d. FIG. 4 shows the differential IUXz taken between a preferred segment of the zero order line. The second delta in particular (IUTz) represents a change in optical density at a time T₂S to the optical density measured at a time T₃, where time T₃ is the end of the test (EOT).

The (IUXz) represents the fibrinogen (FBG) converted between time T₂S and T₂. The (IUTz) represents fibrinogen converted from the time T₂S to the end of the test or T₃.

The first value V1 corresponds to the value determined for the theoretical end of test (TEOT), which, as illustrated in the clotting curve representation in FIG. 4, is where the zero order kinetic line (L) crosses the line y=T₃. The value TEOT is the elapsed time to convert the total instrument units (TIU) at the zero order kinetic rate, which is representative of the fibrinogen in the sample undergoing the conversion to fibrin. In other words, the expression for the first value (V1), or TEOT, is:

V1=TEOT=ZTM/IUXz*IUTz (5)

where ZTM is the time between Tmap (i.e., T₂ shown on FIG. 4) and T2S. ZTM may be generally represented by the following expression:

ZTM=T₂−T₂S (6)

A second value, V2, also referred to as a multiplier, is determined based on the value T₂S. In the expression for the ATFt, the second value, V2, may be obtained by taking the value of the time (T₂S) corresponding to a second time (t2) or the maximum acceleration point (Tmap), and scaling this value. It is illustrated in this embodiment that the multiplier is derived from the natural log base “e”, which is 2.71828, scaled to provide an appropriately decimaled value. The scaling number used in the example set forth for this embodiment is 100. The second value (V2) may be expressed by the following relationship:

V2=T₂S/100e (7)

where T₂S is the maximum acceleration point for the sample, and 100e is the value 100 multiplied by the natural log base “e” (2.71828) or 271.828. The new anticoagulation therapy factor according to the alternate embodiment may be expressed as follows:

nATFt=[(T₂−T₂S)/IUXz*IUTz]*[T₂S/M] (8)

where M represents a multiplier. In the present example, the multiplier M, corresponds to the value 271.828 (which is 100 times the natural log base “e”).

An alternate embodiment of an anticoagulant therapy factor, ATFt2, which does not require the ascertainment of a mean normal prothrombin time (MNPT) or use of an ISI value, is derived using the expression (5), wherein the IUTz is replaced by the expression (IUTz+IULz). In this alternate expression the method is carried out to ascertain the values for Value1 and Value2, in the manner described herein, with Value 1 being obtained through expression (5.1):

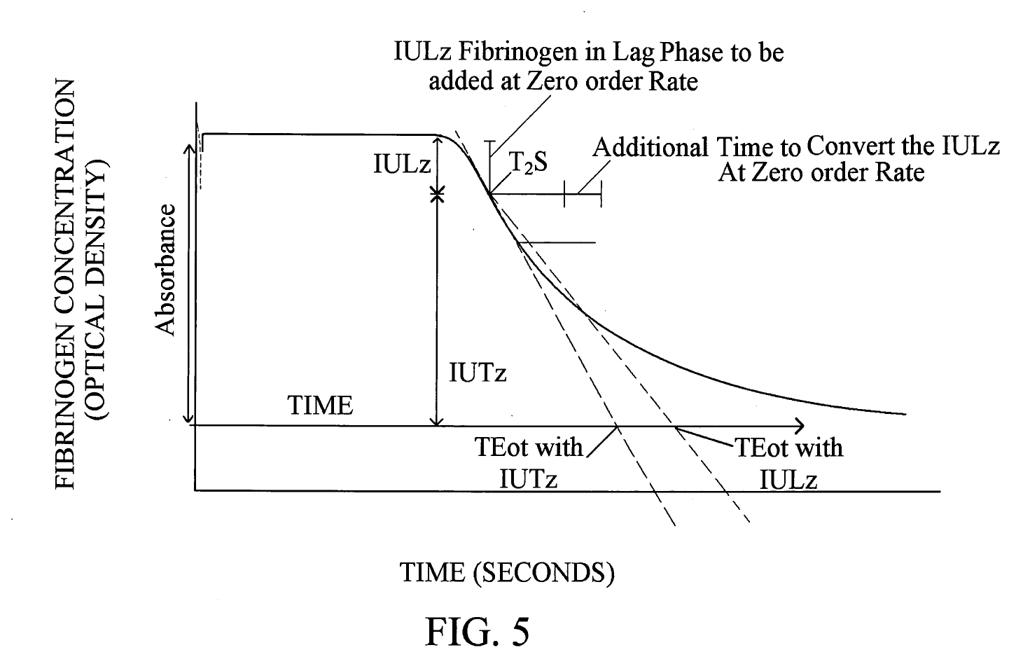

V1=TEOT=ZTM/IUXz*(IUTz+IULz) (5.1)

where IULz is time to convert the lag phase fibrinogen (FBG) measured along the ordinate between T1 and T2S. In expression 5.1, the theoretical end of test (TEOT) is set to include the time to convert the fibrinogen (FBG) in the lag phase of the clotting curve. FIG. 5 illustrates the fibrinogen lag phase and the TEOT obtained from the line L2, and shows the IULz. ATFt2 is expressed by the following:

nATFt2=[(T₂−T₂S)/IUXz*(IUTz+IULz)]*[T₂S/M] (8.1)

The apparatus may comprise a computer which is programmed to record, store and process data. The zero order rate may be determined by ascertaining data from analyzing the sample, and optical density properties. One example of how this may be accomplished is using two arrays, a data array and a sub array. A data array may be ascertained by collecting data over a time interval. In one embodiment, for example, the data array may comprise a sequential list of optical densities, taken of a sample by an optical analytical instrument, such as, for example, a spectrophotometer, for a frequency of time. In the example, the frequency of sample data is taken every 100^{th} of a second. In this embodiment, a computer is programmed to record the optical density of the sample, every 100^{th} of a second. Two values, NOW and THEN, for the data array are provided for ascertaining the Prothrombin Time (PT) (which is the time point T₁), maximum acceleration point (MAP), and end of test point (EOT). Two time definitions may be specified, one being the interval between NOW and THEN on the clotting curve, which may be 2.72 seconds ( 272/100^{th} of a second), the second being the size of the filter used for signal averaging. NOW is the sum of the last 20 optical densities and THEN is the sum of the 10 prior data points 2.72 seconds prior to NOW. A graphical illustration is provided in FIG. 5. As illustrated in FIG. 5, four values are defined: SUM(NOW), SUM(THEN), AVERAGE(NOW) and AVERAGE(THEN). The average is the sum divided by the filter value.

The sub array may be defined as a sequential list of delta absorbance units. This may begin at T₁, the prothrombin time (PT), and continue until the last highest delta absorbance (delta A) has been detected, then continues an additional five (5) seconds to insure the last delta A has been found. A determination of T₂S may be accomplished by locating within the sub array, the first occurrence of when the sub array delta value is greater than or equal to 80% of the highest delta absorbance units. The first derivative is ascertained by computing the difference between (NOW) and (THEN). The PT is ascertained by determining the point prior to the positive difference between AVERAGE(THEN) and AVERAGE(NOW) for a period of 2.72 seconds or 272 ticks. The MAP is the point where the last highest difference between SUM(THEN) and SUM(NOW) has occurred. The computer may be programmed to store this delta A value in the sub array. The EOT may be ascertained by determining the point prior to where the difference between SUM(THEN) and SUM(NOW) is less than one.

Table 2 illustrates examples of samples, identified by ID numbers, along with corresponding data which compares the ATF values obtained for an ATF determined through the prior method, using ISI and INR values (represented as ATFa), an ATF determined through the use of a zero order kinetic reaction using the MNTX (nATFz), and an ATF determined without using the MNXT or ISI (nATFt). The data in table 2 represents universal laboratory data from combined locations for the patients listed. The data is based on analysis of absorbance data, storage of the data by the computer, such as, for example, with a storage device, like a hard drive, and retrieving the data and processing the data. The data, in the example represented in Table 2 was processed using the definitions and NOW and THEN intervals.

TABLE 2

| ID | AINR | GINR | GatfA | GatfZ | GatfT | MINR | MatfA | MatfZ | MatfT |
|---|---|---|---|---|---|---|---|---|---|
| U0047 | 2.10 | 1.70 | 1.76 | 1.74 | 1.62 | 2.00 | 2.08 | 1.78 | 1.68 |
| U0048 | 1.80 | 1.80 | 1.84 | 1.83 | 1.72 | 1.90 | 1.96 | 1.85 | 1.82 |
| U0050 | 1.80 | 1.70 | 1.77 | 1.80 | 1.68 | 1.90 | 2.00 | 1.80 | 1.70 |
| U0056 | 1.60 | 1.50 | 1.54 | 1.54 | 1.40 | 1.80 | 1.83 | 1.61 | 1.48 |
| U0058 | 3.20 | 2.80 | 2.93 | 2.92 | 2.93 | 3.30 | 3.38 | 3.10 | 3.29 |
| U0060 | 2.20 | 2.10 | 2.15 | 2.17 | 2.11 | 2.20 | 2.21 | 2.26 | 2.27 |
| U0062 | 2.80 | 2.60 | 2.69 | 2.72 | 2.69 | 3.00 | 3.19 | 2.86 | 2.91 |
| U0415 | 0.90 | 0.90 | 0.88 | 0.94 | 0.74 | 0.90 | 0.95 | 0.97 | 0.83 |
| U0432 | 1.80 | 1.50 | 1.53 | 1.42 | 1.24 | 1.40 | 1.39 | 1.46 | 1.33 |
| U0436 | 2.40 | 2.40 | 2.57 | 2.24 | 1.99 | 2.40 | 2.41 | 2.28 | 2.17 |
| U0438 | 3.90 | 3.70 | 4.25 | 3.26 | 3.21 | 3.80 | 4.22 | 3.40 | 3.55 |
| U0439 | 2.30 | 2.20 | 2.27 | 1.94 | 1.75 | 2.30 | 2.32 | 2.07 | 2.02 |
| U0440 | 5.80 | 4.80 | 5.41 | 4.33 | 4.50 | 4.60 | 4.84 | 4.55 | 5.18 |
| U0441 | 4.50 | 4.90 | 5.58 | 5.01 | 4.86 | 4.40 | 4.71 | 4.64 | 5.35 |
| U0442 | 1.80 | 1.70 | 1.79 | 1.65 | 1.48 | 1.80 | 1.84 | 1.64 | 1.52 |
| U0800 | 2.00 | 2.00 | 2.02 | 1.78 | 1.64 | 2.10 | 2.11 | 2.12 | 2.09 |
| U0843 | 1.40 | 1.40 | 1.43 | 1.42 | 1.22 | 1.40 | 1.47 | 1.44 | 1.31 |
| U0848 | 1.30 | 1.40 | 1.41 | 1.31 | 1.13 | 1.30 | 1.37 | 1.34 | 1.23 |
| U0849 | 2.40 | 2.30 | 2.44 | 1.94 | 1.77 | 2.30 | 2.38 | 1.98 | 1.93 |
| U0855 | 1.30 | 1.30 | 1.29 | 1.35 | 1.17 | 1.20 | 1.24 | 1.36 | 1.22 |
| U0860 | 1.00 | 1.00 | 0.99 | 1.00 | 0.77 | 1.00 | 0.97 | 1.00 | 0.85 |
| U0861 | 2.80 | 2.90 | 2.98 | 2.70 | 2.58 | 3.00 | 2.99 | 2.88 | 3.00 |
| U0863 | 1.70 | 1.70 | 1.70 | 1.76 | 1.65 | 1.70 | 1.77 | 1.83 | 1.79 |
| U0867 | 3.20 | 2.90 | 3.19 | 2.64 | 2.38 | 3.00 | 3.10 | 2.85 | 2.83 |
| U0875 | 2.20 | 2.00 | 2.16 | 1.80 | 1.60 | 2.00 | 2.02 | 1.81 | 1.71 |
| U1198 | 2.20 | 2.10 | 2.17 | 2.07 | 1.91 | 2.00 | 1.98 | 2.22 | 2.22 |
| U1199 | 2.80 | 3.30 | 3.57 | 2.79 | 2.76 | 3.20 | 3.21 | 2.99 | 3.28 |
| U1201 | 1.90 | 1.90 | 1.95 | 1.76 | 1.62 | 1.80 | 1.84 | 1.82 | 1.80 |
| U1202 | 1.30 | 1.30 | 1.35 | 1.31 | 1.16 | 1.40 | 1.39 | 1.35 | 1.20 |
| U1205 | 1.60 | 1.80 | 1.90 | 1.71 | 1.53 | 1.90 | 1.90 | 1.80 | 1.67 |
| U1207 | 1.90 | 1.90 | 1.96 | 1.68 | 1.49 | 1.90 | 1.87 | 1.78 | 1.61 |
| U1218 | 3.00 | 2.60 | 2.86 | 2.57 | 2.56 | 2.80 | 3.07 | 2.90 | 3.08 |
| U1225 | 2.20 | 2.30 | 2.34 | 2.01 | 1.83 | 2.60 | 2.40 | 2.21 | 2.16 |
| U1230 | 1.30 | 1.40 | 1.45 | 1.47 | 1.32 | 1.40 | 1.45 | 1.50 | 1.45 |
| U1575 | 1.40 | 1.30 | 1.30 | 1.53 | 1.41 | 1.40 | 1.44 | 1.49 | 1.35 |
| U1576 | 2.20 | 2.10 | 2.11 | 2.10 | 2.02 | 2.30 | 2.32 | 2.19 | 2.17 |
| U1579 | 1.50 | 1.70 | 1.72 | 1.64 | 1.49 | 1.80 | 1.81 | 1.61 | 1.44 |
| U1581 | 1.70 | 1.70 | 1.74 | 1.85 | 1.81 | 1.70 | 1.77 | 1.74 | 1.73 |
| U1599 | 2.00 | 1.70 | 1.78 | 2.01 | 1.96 | 2.00 | 2.14 | 2.04 | 1.93 |
| U1600 | 3.50 | 3.30 | 3.39 | 3.58 | 3.63 | 3.90 | 4.21 | 3.37 | 3.64 |
| U1649 | 0.90 | 0.80 | 0.80 | 0.94 | 0.76 | 0.90 | 0.89 | 0.89 | 0.74 |
| U3050 | 2.70 | 2.80 | 3.08 | 2.34 | 2.17 | 2.30 | 2.34 | 2.05 | 2.02 |
| U3077 | 1.30 | 1.40 | 1.44 | 1.34 | 1.17 | 1.30 | 1.28 | 1.31 | 1.16 |
| U3083 | 1.60 | 1.60 | 1.58 | 1.47 | 1.31 | 1.60 | 1.68 | 1.48 | 1.37 |
| U3395 | 2.70 | 3.20 | 3.51 | 2.80 | 2.70 | 2.80 | 2.90 | 2.38 | 2.32 |
| U3398 | 1.50 | 1.70 | 1.77 | 1.60 | 1.47 | 1.60 | 1.65 | 1.61 | 1.47 |
| U3408 | 1.10 | 1.20 | 1.18 | 1.13 | 0.92 | 1.10 | 1.03 | 1.09 | 0.94 |
| U3453 | 1.10 | 1.20 | 1.24 | 1.19 | 0.97 | 1.20 | 1.18 | 1.11 | 1.00 |
| U3456 | 1.10 | 1.00 | 0.96 | 0.99 | 0.81 | 1.00 | 0.98 | 1.04 | 0.90 |
| U3457 | 2.20 | 2.30 | 2.38 | 2.03 | 1.94 | 2.10 | 2.28 | 1.94 | 1.86 |
| U3459 | 2.90 | 2.60 | 2.81 | 2.40 | 2.22 | 2.40 | 2.53 | 2.11 | 2.04 |
| U3724 | 2.70 | 2.40 | 2.47 | 2.16 | 1.95 | 2.60 | 2.72 | 2.31 | 2.25 |
| U4471 | 1.50 | 1.60 | 1.67 | 1.63 | 1.43 | 1.70 | 1.71 | 1.71 | 1.62 |
| U4737 | 2.90 | 2.60 | 2.79 | 2.42 | 2.26 | 2.70 | 2.87 | 2.51 | 2.42 |
| U4752 | 1.40 | 1.50 | 1.55 | 1.47 | 1.26 | 1.50 | 1.48 | 1.46 | 1.33 |
| U4757 | 2.00 | 2.10 | 2.09 | 1.95 | 1.77 | 2.00 | 2.02 | 2.00 | 1.92 |
| U4767 | 2.60 | 2.40 | 2.52 | 2.16 | 1.95 | 2.60 | 2.56 | 2.33 | 2.27 |
| U4772 | 2.50 | 2.70 | 2.78 | 2.59 | 2.58 | 2.80 | 2.84 | 2.55 | 2.56 |
| U4801 | 1.30 | 1.40 | 1.41 | 1.33 | 1.13 | 1.50 | 1.49 | 1.41 | 1.22 |
| U5133 | 0.90 | 0.90 | 0.91 | 0.92 | 0.74 | 1.00 | 0.97 | 0.97 | 0.78 |
| U5158 | 5.50 | 5.10 | 5.90 | 5.34 | 5.64 | 6.00 | 6.57 | 6.50 | 7.00 |
| U5169 | 2.60 | 2.90 | 3.16 | 3.14 | 3.09 | 3.20 | 3.35 | 3.35 | 3.67 |
| U5173 | 1.10 | 1.20 | 1.17 | 1.19 | 1.02 | 1.20 | 1.21 | 1.16 | 1.03 |
| U5175 | 1.70 | 1.80 | 1.86 | 1.85 | 1.67 | 1.90 | 1.92 | 1.82 | 1.70 |
| U5178 | 2.30 | 2.20 | 2.28 | 2.02 | 1.79 | 2.60 | 2.85 | 2.03 | 2.01 |
| U5183 | 2.90 | 2.60 | 2.83 | 2.43 | 2.23 | 3.60 | 3.86 | 2.88 | 3.01 |
| U5190 | 2.80 | 2.70 | 2.82 | 2.85 | 2.70 | 3.20 | 3.36 | 3.00 | 3.15 |
| U5193 | 3.10 | 3.00 | 3.13 | 2.93 | 2.81 | 3.60 | 3.73 | 3.33 | 3.30 |
| U5565 | 2.70 | 3.20 | 3.34 | 3.16 | 3.04 | 3.50 | 3.48 | 3.31 | 3.50 |
| U5589 | 1.60 | 1.80 | 1.86 | 1.69 | 1.52 | 1.90 | 1.96 | 1.64 | 1.44 |
| U5591 | 2.00 | 2.20 | 2.33 | 2.16 | 1.98 | 2.30 | 2.28 | 2.19 | 2.24 |
| U5592 | 1.10 | 1.20 | 1.23 | 1.26 | 1.09 | 1.40 | 1.35 | 1.49 | 1.37 |
| U5593 | 1.70 | 1.80 | 1.89 | 1.76 | 1.55 | 1.80 | 1.85 | 1.76 | 1.70 |
| U5594 | 2.30 | 2.60 | 2.79 | 2.84 | 2.81 | 2.80 | 2.84 | 2.85 | 2.96 |
| U5597 | 3.30 | 3.30 | 3.64 | 3.25 | 2.96 | 4.10 | 4.03 | 3.85 | 4.08 |
| U5992 | 1.40 | 1.40 | 1.42 | 1.45 | 1.29 | 1.30 | 1.37 | 1.37 | 1.30 |
| U5993 | 1.00 | 0.90 | 0.94 | 1.03 | 0.84 | 1.00 | 0.98 | 1.03 | 0.84 |
| U6017 | 1.00 | 0.90 | 0.95 | 0.99 | 0.77 | 0.90 | 0.89 | 0.97 | 0.79 |
| U6047 | 2.30 | 2.30 | 2.36 | 2.17 | 1.97 | 2.20 | 2.28 | 2.23 | 2.22 |
| U6056 | 1.00 | 1.00 | 1.01 | 1.03 | 0.87 | 1.00 | 1.01 | 1.02 | 0.85 |
| U6060 | 1.90 | 2.10 | 2.17 | 2.10 | 1.94 | 2.30 | 2.00 | 2.16 | 2.12 |
| U6065 | 3.10 | 2.80 | 2.93 | 2.77 | 2.60 | 3.00 | 3.13 | 2.74 | 2.76 |
| U6928 | 1.20 | 1.20 | 1.17 | 1.34 | 1.17 | 1.20 | 1.24 | 1.22 | 1.05 |
| U6929 | 1.20 | 1.20 | 1.20 | 1.23 | 1.06 | 1.20 | 1.19 | 1.15 | 0.98 |
| U6936 | 2.40 | 2.50 | 2.45 | 3.02 | 3.15 | 2.60 | 2.61 | 2.51 | 2.60 |
| U6938 | 2.10 | 2.10 | 2.12 | 2.30 | 2.22 | 2.30 | 2.26 | 2.25 | 2.21 |
| U6951 | 1.50 | 1.50 | 1.51 | 1.59 | 1.42 | 1.60 | 1.66 | 1.49 | 1.36 |
| U6972 | 2.40 | 2.40 | 2.47 | 2.57 | 2.49 | 2.80 | 2.84 | 2.54 | 2.51 |
| U6977 | 1.30 | 1.30 | 1.34 | 1.35 | 1.19 | 1.30 | 1.37 | 1.23 | 1.08 |
| U6987 | 5.10 | 4.50 | 4.43 | 5.29 | 5.42 | 5.70 | 5.44 | 6.16 | 6.82 |
| U7316 | 1.20 | 1.10 | 1.15 | 1.28 | 1.14 | 1.30 | 1.28 | 1.26 | 1.11 |
| U7317 | 2.00 | 1.60 | 1.68 | 1.66 | 1.56 | 1.90 | 1.90 | 1.68 | 1.56 |
| U7318 | 2.80 | 2.70 | 2.86 | 2.71 | 2.57 | 3.30 | 3.40 | 2.70 | 2.72 |
| U7320 | 2.00 | 1.90 | 1.92 | 2.17 | 2.13 | 2.00 | 2.06 | 2.12 | 2.13 |
| U7321 | 1.50 | 1.40 | 1.38 | 1.59 | 1.50 | 1.60 | 1.60 | 1.61 | 1.51 |
| U7322 | 1.80 | 1.70 | 1.72 | 1.63 | 1.46 | 1.70 | 1.76 | 1.55 | 1.42 |
| U7324 | 1.30 | 1.20 | 1.25 | 1.33 | 1.17 | 1.40 | 1.40 | 1.30 | 1.13 |
| U7440 | 2.60 | 3.00 | 2.98 | 2.90 | 2.89 | 3.00 | 3.01 | 3.05 | 3.37 |
| U7443 | 2.00 | 2.00 | 2.03 | 1.87 | 1.73 | 2.10 | 2.17 | 1.90 | 1.79 |
| U7458 | 1.40 | 1.40 | 1.43 | 1.38 | 1.20 | 1.40 | 1.40 | 1.40 | 1.26 |
| U7465 | 9.70 | 7.40 | 8.12 | 6.47 | 7.80 | 7.10 | 7.54 | 7.06 | 7.63 |
| U7469 | 1.10 | 1.10 | 1.11 | 1.11 | 0.86 | 1.20 | 1.14 | 1.10 | 0.90 |
| U7470 | 3.20 | 3.40 | 3.65 | 3.27 | 3.12 | 3.60 | 3.67 | 3.62 | 3.70 |
| U7707 | 2.20 | 2.20 | 2.27 | 2.34 | 2.28 | 2.30 | 2.29 | 2.23 | 2.22 |
| U7708 | 1.60 | 1.60 | 1.60 | 1.73 | 1.61 | 1.70 | 1.73 | 1.71 | 1.62 |
| U7710 | 2.30 | 2.50 | 2.64 | 2.71 | 2.73 | 2.70 | 2.85 | 2.75 | 2.96 |
| U7713 | 1.40 | 1.60 | 1.59 | 1.57 | 1.50 | 1.60 | 1.64 | 1.58 | 1.48 |
| U7724 | 2.40 | 2.40 | 2.47 | 2.62 | 2.65 | 2.70 | 2.73 | 2.75 | 2.84 |
| U7727 | 1.70 | 1.70 | 1.73 | 1.78 | 1.68 | 1.90 | 1.90 | 1.91 | 1.86 |
| U7738 | 2.40 | 2.30 | 2.45 | 2.27 | 2.21 | 2.40 | 2.54 | 2.29 | 2.32 |
| U7794 | 1.90 | 1.80 | 1.91 | 1.72 | 1.58 | 1.70 | 1.78 | 1.71 | 1.55 |
| U8080 | 3.10 | 3.60 | 3.63 | 3.41 | 3.54 | 3.30 | 3.33 | 3.18 | 3.34 |
| U8087 | 1.90 | 1.90 | 1.95 | 1.80 | 1.62 | 1.90 | 1.91 | 1.79 | 1.74 |
| U8092 | 1.70 | 1.70 | 1.76 | 1.67 | 1.49 | 1.90 | 1.93 | 1.67 | 1.57 |
| U8210 | 2.60 | 2.90 | 3.04 | 2.72 | 2.63 | 2.70 | 2.77 | 2.54 | 2.56 |
| U8221 | 3.20 | 3.70 | 3.99 | 3.42 | 3.35 | 3.50 | 3.47 | 3.24 | 3.46 |
| U8555 | 2.60 | 2.40 | 2.54 | 2.56 | 2.52 | 2.90 | 3.09 | 2.57 | 2.56 |
| U8558 | 2.30 | 2.20 | 2.26 | 2.16 | 2.15 | 2.30 | 2.33 | 2.31 | 2.35 |
| U8559 | 1.60 | 1.40 | 1.45 | 1.42 | 1.24 | 1.60 | 1.65 | 1.45 | 1.28 |
| U8563 | 2.20 | 2.30 | 2.30 | 2.32 | 2.30 | 2.40 | 2.43 | 2.34 | 2.42 |
| U8570 | 1.20 | 1.20 | 1.20 | 1.34 | 1.23 | 1.20 | 1.21 | 1.35 | 1.25 |
| U8575 | 0.90 | 0.80 | 0.84 | 0.96 | 0.80 | 0.90 | 0.89 | 0.95 | 0.78 |
| U9031 | 2.10 | 2.40 | 2.33 | 2.42 | 2.42 | 2.60 | 2.38 | 2.34 | 2.35 |
| U9032 | 1.70 | 1.70 | 1.75 | 1.78 | 1.58 | 1.90 | 1.93 | 1.68 | 1.53 |
| U9034 | 3.00 | 2.90 | 2.82 | 3.79 | 3.97 | 3.40 | 3.37 | 3.49 | 3.80 |
| U9039 | 2.70 | 3.00 | 3.17 | 2.99 | 3.03 | 3.20 | 3.20 | 3.12 | 3.27 |
| U9040 | 1.40 | 1.40 | 1.44 | 1.36 | 1.20 | 1.40 | 1.39 | 1.33 | 1.15 |
| U9049 | 3.50 | 3.30 | 3.46 | 3.33 | 3.45 | 3.60 | 3.77 | 3.33 | 3.72 |
| U9055 | 2.40 | 2.10 | 2.14 | 2.15 | 2.04 | 2.40 | 2.39 | 2.15 | 2.13 |

A statistical comparison of the above data from Table 2 is presented below in Tables 4 and 5. The value AINR in Table 2 represents the INR value obtained pursuant to the World Health Organization (WHO), using expressions (A) and (B) above. GINR and MINR correspond to INR values used to determine the comparison data set forth in Tables 4 and 5.

The determination of the new anticoagulant therapy factor (ATFt) may be carried out with a computer. According to one example, the gathering, storing, and manipulation of the data generally illustrated in FIG. 4, may be accomplished by computer 30 of FIG. 1 that receives digital voltage values converted, by the A/D converter 26, from analog voltage quantities of the photocell 10 detection means.

In accordance with one embodiment, the IBM-compatible computer 30 of FIG. 1 stores and manipulates these digital values corresponding to related data of FIG. 4 and may be programmed as follows:
(a) a sample of blood where the plasma is available, such as, for example, a sample of citrated blood, is obtained and placed in an appropriate container, the computer 30, as well as the recorder 28, sequentially records voltage values for a few seconds before injection of thromboplastin. As previously discussed, thromboplastin (tissue factor) is one of the factors in the human body that causes blood to clot. Prothrombin is another. Fibrinogen is yet another. Before injection of the thromboplastin, the output from the A/D converter 26 is relatively constant. When thromboplastin is injected into the plasma sample in the container, a significant and abrupt change occurs in the recorded voltage values of both the computer 30 and the recorder 28. This abrupt change is recognized by both the recorder 28 and, more importantly, by the computer 30 which uses such recognition to establish Tₒ. The computer 30 may be programmed so as to correlate the digital quantities of the A/D converter 26 to the analog output of the detector means photocell 10 which, in turn, is directly correlatable to the fibrinogen (FBG) concentration g/l of the sample of blood discussed with reference to FIG. 3;
(b) the computer 30 may be programmed to look for a digital quantity representative of a critical quantity c₁, and when such occurs, record its instant time T₁. (The time span between Tₒ and T₁ is the prothrombin time (PT), and has an normal duration of about 12 seconds, but may be greater than 30 seconds);
(c) following the detection of the quantity c₁, the computer 30 may be programmed to detect for the acceleration of fibrinogen (FBG) to fibrin conversion. The computer 30 is programmed to detect the maximum acceleration quantity, c_{MAP} or c_{T2} as illustrated in FIG. 3, and its corresponding time of occurrence t_{MAP}, which is T₂ in FIG. 3.
(d) the computer detects a quantity c_{EOT} occurring at time t_{EOT}. Typically; it is important that the rate of fibrin formation increase for at least 1.5 seconds following the occurrence of (T₁); the computer determines a theoretical end of test (TEOT) based on the determination of the zero order kinetic rate. The computer may be programmed to determine the zero order rate, which is expressed as a Line (L) in FIG. 4. The TEOT may be determined by the corresponding time value (TEOT) along the line L which corresponds with the quantity c_{EOT} (i.e., that quantity corresponding to the time, T₃).
(e) following the detection of the maximum acceleration quantity c_{T2} (also representing c_{MAP}) and the time T₂ (also representing t_{MAP}) both of which define the maximum acceleration point (MAP), and the TEOT, the computer is programmed to determine a new fibrinogen transformation rate (nFTR) covering a predetermined range starting prior to the maximum acceleration point (MAP) and ending after the maximum acceleration point (MAP). The elapsed time from T₀ to T₂ (which is t_{MAP}) is the time to maximum acceleration (TMA), shown in FIG. 4, and is represented by TX (i.e., time to MAP);
The new fibrinogen transformation rate (nFTR) has an upwardly rising (increasing quantities) slope prior to the maximum acceleration point (MAP) and, conversely, has a downwardly falling (decreasing quantities) slope after the maximum acceleration point (MAP).
The computer 30 is programmed to ascertain the value for the time to start (T₂S) which corresponds with the time at which the simulated zero order kinetic rate begins.
(f) following the detection of the acceleration of fibrinogen conversion to detect the start time T₂S, the computer 30 is programmed to detect for a deceleration of the fibrinogen conversion, wherein the fibrinogen concentration decreases from a predetermined quantity c_{MAP} to a predetermined quantity c_{EOT} having a value which is about equal but less than the first quantity c₁. The computer is programmed to ascertain a first delta (IUTz), by determining the difference between the quantity c_{T2S} and the quantity c_{EOT}; and a second delta (IUXz) by determining the difference between the quantity c_{T2S} and the quantity c_{2 (or CMAP)}; the computer also determines the value ZTM by determining the difference between the time T₂ (which is Tmap) and the time T₂S;
(g) the computer 30 manipulates the collected data of (a); (b); (c); (d), (e) and (f) above, to determine the new fibrinogen transfer rate (nFTR). The nFTR may be arrived at based on the principle that if a required amount (e.g., 0.05 g/l) of fibrinogen concentration c₁ is first necessary to detect a clot point (t₁); then when the fibrinogen concentration (c_{EOT}) becomes less than the required amount c₁, which occurs at time (t_{EOT}), the fibrinogen end point has been reached. More particularly, the required fibrinogen concentration c₁ is the starting point of fibrinogen conversion of the clotting process and the less than required fibrinogen concentration c_{EOT} is the end point of the fibrinogen conversion of the clotting process.
(h) the duration of the fibrinogen conversion of the clotting process of the present invention is defined by the zero order time period between TEOT and T₂S and is generally indicated in FIG. 3 as IUTz. The difference between the corresponding concentrations c_{T2S} and cT2 is used to define a delta IUXz. The computer now has the information needed to determine the TEOT, which is expressed by the following formula:

TEOT=ZTM/IUXz*IUTz (5)
The value TEOT may be assigned VALUE 1;
(i) data collected is manipulated by the computer 30 to calculate a second value, VALUE 2, using T₂S and a multiplier M (which in this example, in expression 7 below, is a fraction). The computer may be programmed to use as a multiplier a value based on the natural log base “e” (which is 2.71828), scaled by a scaling value. Here, the scaling value is 100, and the multiplier may be expressed as follows:

M=100e (9)
VALUE 2 is determined using the information which the computer has ascertained and stored, by the following expression:

VALUE 2=T2S/100e (7)

The data may be ascertained and stored in the computer for reference.
(j) the computer 30 now has the information needed to determine the nATFt, which typically is expressed as:

nATFt=VALUE 1*VALUE 2 (4)

The computer 30 may be used to manipulate and derive the quantities of expression (4) to determine a new anticoagulant therapy factor nATFt utilizing known programming routines and techniques. The data collected by a computer 30 may be used to manipulate and derive the new anticoagulant therapy factor (nATFt) of expression (4). Similarly, one skilled in the art, using known mathematical techniques may derive the theoretical end of test TEOT of expression (5) and the second value VALUE 2 of expression (7) which, in turn, are used to determine the new anticoagulant therapy (nATFt) of expression (4). In the nATFt determination, the determination is based on the patient's own sample, and does not rely on the determination of normal prothrombin times for the reagent used (e.g., thromboplastin, innovin or the like). With the nATFt, no longer does the accuracy of the quantities determined depend, in whole or part, on the number of specimens used, that is, the number of stable (or presumed stable) patients.

The new anticoagulation therapy factor (nATFt) does not require an ISI value, as was previously used to determine anticoagulation therapy factors. The new anticoagulation therapy factor (nATFt) uses for its ascertainment the values extracted from the clotting curve (see FIG. 4), in particular T₂S, Tmap, TEOT, c_{T2S}, cmap and ceot. In determining the new anticoagulant therapy factor (nATFt), the ISI is not required, nor is the MNPT, or the need to obtain and calculate the prothrombin times (PT's) for 20 presumed normal patients. In carrying out coagulation studies, the new anticoagulant therapy factor (nATFt) may replace INR in anticoagulant therapy management. In addition, using the sample from the patient, the computer 30 has knowledge of the values obtained for the fibrinogen reaction, to ascertain the (nATFt).

It should now be appreciated that the present invention provides an apparatus and method for obtaining a new anticoagulant therapy factor (nATF) without encountering the complications involved with obtaining the prior art quantities International Normalized Ratio (INR) and International Sensitivity Index (ISI).

The new anticoagulant therapy factor (nATFt) preferably is a replacement for the International Normalized Ratio (INR). Existing medical literature, instrumentation, and methodologies are closely linked to the International Normalized Ratio (INR). The nATFt was compared for correlation with the INR by comparative testing, to INR quantities, even with the understanding that the INR determination may have an error of about +/−15%, at a 95% confidence interval, which needs to be taken into account to explain certain inconsistencies.

The hereinbefore description of the new anticoagulant therapy factor (nATFt) does correlate at least as well as, and preferably better than, studies carried out using the International Normalized Ratio (INR). For some comparisons, see the tables below, and in particular Table 4 and Table 5.

Table 3 (Part A) and Table 3 (Part B) provide corresponding data for a coagulation study. In Table 3 (Part A and B), the following references are used:

| Column | Label | Definition |
|---|---|---|
| A | ID | Sample ID |
| B | OD@T₂S | OD at the start of Zero Order Kinetic |
| C | OD@Map | OD at the Maximum Acceleration Point |
| | | (MAP) |
| D | OD@Eot | OD at the END OF TEST (Eot) |
| E | ΔT₂SMap | Delta of Column B and C creating the IUXz |
| F | ΔT₂SEot | Delta of Column B and D creating the IUTz |
| G | FTR od | Ratio of Column E divided by F |
| | | The FTR od is subtracted from 2 creating the |
| | | Exponent that replaces the ISI |
| H | Time@T₂S | Time at the start of Zero Order Kinetics |
| I | Time@Map | Time at the Maximum Acceleration Point |
| | | (MAP) |
| J | Time@TEot | Time at the Theoretical End of Test (TEOT) |
| K | ΔT₂SMap | Delta of Column H and I creating the IUXz |
| | | (and ZTM) |
| L | ΔT₂STEot | Delta of Column H and J creating the IUTz |
| M | FTR Time | Ration of Column K divided by L |

TABLE 3 (Part A)

| ID | OD@T2S | OD@Map | OD@Eot | ΔT2SMap | ΔT2SEot |
|---|---|---|---|---|---|
| A001 | 3719 | 3707 | 3664 | 12 | 55 |
| A002 | 3713 | 3704 | 3686 | 9 | 27 |
| A003 | 3729 | 3720 | 3705 | 9 | 24 |
| A004 | 3708 | 3696 | 3663 | 12 | 45 |
| A005 | 3727 | 3715 | 3700 | 12 | 27 |
| A007 | 3725 | 3718 | 3698 | 7 | 27 |
| A008 | 3714 | 3693 | 3646 | 21 | 68 |
| A009 | 3727 | 3716 | 3697 | 11 | 30 |
| A010 | 3727 | 3714 | 3701 | 13 | 26 |
| A011 | 3690 | 3676 | 3647 | 14 | 43 |
| A012 | 3728 | 3716 | 3695 | 12 | 33 |
| A013 | 3715 | 3690 | 3641 | 25 | 74 |
| A014 | 3717 | 3708 | 3694 | 9 | 23 |
| A015 | 3726 | 3718 | 3706 | 8 | 20 |
| A016 | 3722 | 3715 | 3678 | 7 | 44 |
| A017 | 3720 | 3707 | 3681 | 13 | 39 |
| A018 | 3723 | 3709 | 3697 | 14 | 26 |
| A019 | 3716 | 3695 | 3653 | 21 | 63 |
| A020 | 3727 | 3716 | 3698 | 11 | 29 |
| A021 | 3727 | 3720 | 3694 | 7 | 33 |
| A022 | 3717 | 3700 | 3667 | 17 | 50 |
| A023 | 3719 | 3706 | 3663 | 13 | 56 |
| A024 | 3717 | 3702 | 3661 | 15 | 56 |
| A025 | 3731 | 3727 | 3716 | 4 | 15 |
| A026 | 3717 | 3705 | 3673 | 12 | 44 |
| A027 | 3714 | 3698 | 3667 | 16 | 47 |
| A028 | 3713 | 3696 | 3651 | 17 | 62 |
| A029 | 3712 | 3691 | 3647 | 21 | 65 |
| A030 | 3716 | 3695 | 3635 | 21 | 81 |
| A031 | 3715 | 3704 | 3687 | 11 | 28 |
| A032 | 3716 | 3710 | 3675 | 6 | 41 |
| A033 | 3718 | 3704 | 3671 | 14 | 47 |
| A034 | 3721 | 3705 | 3674 | 16 | 47 |
| A035 | 3723 | 3715 | 3699 | 8 | 24 |
| A036 | 3722 | 3710 | 3681 | 12 | 41 |
| A037 | 3715 | 3700 | 3669 | 15 | 46 |
| A038 | 3722 | 3707 | 3686 | 15 | 36 |
| A039 | 3721 | 3712 | 3698 | 9 | 23 |
| A040 | 3720 | 3706 | 3664 | 14 | 56 |
| A041 | 3711 | 3695 | 3638 | 16 | 73 |
| A042 | 3722 | 3709 | 3687 | 13 | 35 |
| A044 | 3723 | 3709 | 3683 | 14 | 40 |
| A045 | 3712 | 3697 | 3647 | 15 | 65 |
| A047 | 3716 | 3697 | 3668 | 19 | 48 |
| A048 | 3720 | 3708 | 3682 | 12 | 38 |
| A049 | 3725 | 3711 | 3690 | 14 | 35 |
| A050 | 3724 | 3712 | 3685 | 12 | 39 |
| A051 | 3705 | 3688 | 3634 | 17 | 71 |
| A052 | 3725 | 3714 | 3687 | 11 | 38 |
| A053 | 3724 | 3717 | 3696 | 7 | 28 |
| A054 | 3715 | 3701 | 3679 | 14 | 36 |
| A055 | 3718 | 3684 | 3627 | 34 | 91 |
| A056 | 3710 | 3689 | 3624 | 21 | 86 |
| A057 | 3709 | 3701 | 3683 | 8 | 26 |
| A058 | 3725 | 3710 | 3669 | 15 | 56 |
| A059 | 3722 | 3712 | 3696 | 10 | 26 |
| A060 | 3719 | 3712 | 3698 | 7 | 21 |
| A061 | 3720 | 3708 | 3680 | 12 | 40 |
| A062 | 3719 | 3701 | 3651 | 18 | 68 |
| A063 | 3728 | 3715 | 3697 | 13 | 31 |
| A064 | 3718 | 3707 | 3685 | 11 | 33 |
| A065 | 3721 | 3704 | 3680 | 17 | 41 |
| A066 | 3727 | 3717 | 3707 | 10 | 20 |
| A067 | 3708 | 3689 | 3641 | 19 | 67 |
| A068 | 3726 | 3712 | 3686 | 14 | 40 |
| A069 | 3719 | 3715 | 3695 | 4 | 24 |
| A070 | 3716 | 3705 | 3671 | 11 | 45 |
| A071 | 3714 | 3696 | 3660 | 18 | 54 |
| A072 | 3713 | 3693 | 3646 | 20 | 67 |
| A073 | 3707 | 3686 | 3639 | 21 | 68 |
| A074 | 3699 | 3684 | 3665 | 15 | 34 |
| A075 | 3734 | 3730 | 3726 | 4 | 8 |
| A076 | 3719 | 3704 | 3665 | 15 | 54 |
| A077 | 3718 | 3694 | 3634 | 24 | 84 |
| A078 | 3723 | 3707 | 3684 | 16 | 39 |
| A080 | 3729 | 3712 | 3637 | 17 | 92 |
| A081 | 3710 | 3694 | 3626 | 16 | 84 |
| A082 | 3716 | 3703 | 3654 | 13 | 62 |
| A083 | 3720 | 3710 | 3686 | 10 | 34 |
| A084 | 3731 | 3721 | 3667 | 10 | 64 |
| A085 | 3727 | 3704 | 3675 | 23 | 52 |
| A086 | 3717 | 3699 | 3650 | 18 | 67 |
| A087 | 3715 | 3694 | 3654 | 21 | 61 |
| A088 | 3704 | 3681 | 3630 | 23 | 74 |
| A089 | 3723 | 3714 | 3687 | 9 | 36 |
| A090 | 3714 | 3685 | 3588 | 29 | 126 |
| A091 | 3724 | 3710 | 3659 | 14 | 65 |
| A092 | 3696 | 3657 | 3582 | 39 | 114 |
| A093 | 3730 | 3716 | 3693 | 14 | 37 |
| A094 | 3720 | 3708 | 3676 | 12 | 44 |
| A095 | 3710 | 3689 | 3638 | 21 | 72 |
| A096 | 3725 | 3717 | 3700 | 8 | 25 |
| A097 | 3721 | 3713 | 3692 | 8 | 29 |
| A098 | 3716 | 3696 | 3659 | 20 | 57 |
| A099 | 3720 | 3712 | 3685 | 8 | 35 |
| A100 | 3709 | 3685 | 3625 | 24 | 84 |
| A101 | 3727 | 3715 | 3690 | 12 | 37 |
| A102 | 3722 | 3708 | 3661 | 14 | 61 |
| A103 | 3714 | 3693 | 3640 | 21 | 74 |
| A104 | 3719 | 3705 | 3682 | 14 | 37 |
| A105 | 3725 | 3706 | 3660 | 19 | 65 |
| A107 | 3720 | 3707 | 3660 | 13 | 60 |
| A108 | 3731 | 3723 | 3709 | 8 | 22 |
| A109 | 3727 | 3711 | 3689 | 16 | 38 |
| A110 | 3719 | 3693 | 3635 | 26 | 84 |
| A111 | 3723 | 3701 | 3667 | 22 | 56 |
| A112 | 3714 | 3695 | 3614 | 19 | 100 |
| A113 | 3717 | 3702 | 3664 | 15 | 53 |
| A114 | 3711 | 3687 | 3655 | 24 | 56 |
| A115 | 3716 | 3697 | 3652 | 19 | 64 |
| A116 | 3726 | 3717 | 3698 | 9 | 28 |
| A117 | 3710 | 3688 | 3630 | 22 | 80 |
| A118 | 3729 | 3721 | 3699 | 8 | 30 |
| A119 | 3729 | 3716 | 3679 | 13 | 50 |
| A120 | 3722 | 3713 | 3688 | 9 | 34 |
| A121 | 3730 | 3722 | 3704 | 8 | 26 |
| A122 | 3713 | 3688 | 3650 | 25 | 63 |
| A123 | 3729 | 3721 | 3704 | 8 | 25 |
| A124 | 3721 | 3712 | 3696 | 9 | 25 |
| A125 | 3683 | 3668 | 3600 | 15 | 83 |
| A126 | 3736 | 3723 | 3714 | 13 | 22 |
| A127 | 3715 | 3703 | 3640 | 12 | 75 |
| A128 | 3723 | 3714 | 3682 | 9 | 41 |
| A129 | 3728 | 3715 | 3677 | 13 | 51 |
| A130 | 3715 | 3700 | 3656 | 15 | 59 |
| A131 | 3723 | 3711 | 3690 | 12 | 33 |
| A132 | 3720 | 3700 | 3665 | 20 | 55 |
| A133 | 3728 | 3706 | 3673 | 22 | 55 |
| A134 | 3725 | 3696 | 3667 | 29 | 58 |
| A135 | 3717 | 3703 | 3676 | 14 | 41 |
| A136 | 3725 | 3712 | 3659 | 13 | 66 |
| A137 | 3712 | 3691 | 3662 | 21 | 50 |
| A138 | 3714 | 3691 | 3641 | 23 | 73 |
| A139 | 3717 | 3700 | 3642 | 17 | 75 |
| A140 | 3710 | 3690 | 3642 | 20 | 68 |
| A141 | 3715 | 3698 | 3661 | 17 | 54 |
| A142 | 3729 | 3719 | 3706 | 10 | 23 |
| A143 | 3726 | 3709 | 3693 | 17 | 33 |
| A144 | 3709 | 3693 | 3641 | 16 | 68 |
| A145 | 3704 | 3688 | 3639 | 16 | 65 |
| A146 | 3718 | 3706 | 3664 | 12 | 54 |
| A147 | 3713 | 3698 | 3661 | 15 | 52 |
| A148 | 3714 | 3701 | 3646 | 13 | 68 |
| A149 | 3711 | 3692 | 3653 | 19 | 58 |
| A150 | 3701 | 3678 | 3608 | 23 | 93 |
| A151 | 3701 | 3668 | 3587 | 33 | 114 |
| A152 | 3717 | 3706 | 3683 | 11 | 34 |
| A153 | 3691 | 3669 | 3596 | 22 | 95 |
| A154 | 3706 | 3690 | 3645 | 16 | 61 |
| A155 | 3724 | 3703 | 3667 | 21 | 57 |
| A156 | 3717 | 3711 | 3688 | 6 | 29 |
| A157 | 3717 | 3702 | 3678 | 15 | 39 |
| A158 | 3723 | 3715 | 3689 | 8 | 34 |
| A159 | 3714 | 3696 | 3652 | 18 | 62 |
| A160 | 3717 | 3690 | 3655 | 27 | 62 |
| A161 | 3720 | 3713 | 3676 | 7 | 44 |
| A162 | 3722 | 3706 | 3653 | 16 | 69 |
| A163 | 3725 | 3715 | 3683 | 10 | 42 |
| A164 | 3721 | 3712 | 3685 | 9 | 36 |
| A165 | 3707 | 3693 | 3636 | 14 | 71 |
| A166 | 3704 | 3683 | 3631 | 21 | 73 |
| A167 | 3718 | 3712 | 3690 | 6 | 28 |
| A168 | 3722 | 3700 | 3669 | 22 | 53 |
| A169 | 3705 | 3694 | 3624 | 11 | 81 |
| A170 | 3717 | 3704 | 3680 | 13 | 37 |
| A171 | 3721 | 3699 | 3666 | 22 | 55 |
| A172 | 3726 | 3719 | 3691 | 7 | 35 |
| A173 | 3718 | 3708 | 3680 | 10 | 38 |
| A174 | 3707 | 3692 | 3648 | 15 | 59 |
| A175 | 3689 | 3671 | 3642 | 18 | 47 |
| A176 | 3724 | 3711 | 3671 | 13 | 53 |
| A177 | 3721 | 3710 | 3689 | 11 | 32 |
| A178 | 3716 | 3700 | 3655 | 16 | 61 |
| A179 | 3717 | 3707 | 3672 | 10 | 45 |
| A180 | 3718 | 3706 | 3686 | 12 | 32 |
| A181 | 3722 | 3703 | 3676 | 19 | 46 |
| A182 | 3716 | 3706 | 3667 | 10 | 49 |
| A183 | 3711 | 3703 | 3689 | 8 | 22 |
| A184 | 3717 | 3705 | 3661 | 12 | 56 |
| A185 | 3711 | 3694 | 3639 | 17 | 72 |
| A186 | 3721 | 3675 | 3620 | 46 | 101 |
| A187 | 3715 | 3704 | 3668 | 11 | 47 |
| A188 | 3717 | 3703 | 3672 | 14 | 45 |
| A189 | 3709 | 3689 | 3658 | 20 | 51 |
| A190 | 3718 | 3709 | 3688 | 9 | 30 |
| A191 | 3725 | 3717 | 3696 | 8 | 29 |
| A192 | 3722 | 3714 | 3691 | 8 | 31 |
| A193 | 3727 | 3718 | 3685 | 9 | 42 |
| A194 | 3720 | 3710 | 3688 | 10 | 32 |
| A195 | 3691 | 3667 | 3589 | 24 | 102 |
| A196 | 3718 | 3707 | 3673 | 11 | 45 |
| A197 | 3706 | 3692 | 3637 | 14 | 69 |
| A198 | 3717 | 3707 | 3692 | 10 | 25 |
| A199 | 3720 | 3705 | 3684 | 15 | 36 |
| A200 | 3718 | 3709 | 3686 | 9 | 32 |
| A201 | 3725 | 3713 | 3681 | 12 | 44 |
| A202 | 3723 | 3713 | 3694 | 10 | 29 |
| A203 | 3715 | 3704 | 3670 | 11 | 45 |
| A204 | 3723 | 3713 | 3697 | 10 | 26 |
| A205 | 3717 | 3706 | 3674 | 11 | 43 |
| A207 | 3710 | 3702 | 3668 | 8 | 42 |
| A208 | 3722 | 3708 | 3680 | 14 | 42 |
| A209 | 3725 | 3709 | 3682 | 16 | 43 |
| A210 | 3724 | 3714 | 3688 | 10 | 36 |
| A211 | 3712 | 3694 | 3637 | 18 | 75 |
| A212 | 3727 | 3711 | 3689 | 16 | 38 |
| A213 | 3724 | 3705 | 3652 | 19 | 72 |
| A214 | 3727 | 3715 | 3687 | 12 | 40 |
| A215 | 3715 | 3703 | 3668 | 12 | 47 |
| A216 | 3722 | 3707 | 3667 | 15 | 55 |
| A217 | 3716 | 3695 | 3630 | 21 | 86 |
| A218 | 3699 | 3665 | 3583 | 34 | 116 |
| A219 | 3727 | 3716 | 3699 | 11 | 28 |
| A220 | 3717 | 3704 | 3674 | 13 | 43 |
| A222 | 3713 | 3704 | 3684 | 9 | 29 |
| A223 | 3724 | 3715 | 3695 | 9 | 29 |
| A224 | 3718 | 3703 | 3676 | 15 | 42 |
| A225 | 3721 | 3707 | 3683 | 14 | 38 |

TABLE 3 (Part B)

| ID | FTR od | Time@T2S | Time@Map | Time@TEot | ΔT2SMap | ΔT2STEot | FTR time | FTR od |
|---|---|---|---|---|---|---|---|---|
| A001 | 0.218 | 2211 | 2366 | 2921 | 155 | 710 | 0.218 | 0.218 |
| A002 | 0.333 | 2279 | 2464 | 2834 | 185 | 555 | 0.333 | 0.333 |
| A003 | 0.375 | 2329 | 2523 | 2846 | 194 | 517 | 0.375 | 0.375 |
| A004 | 0.267 | 1975 | 2107 | 2470 | 132 | 495 | 0.267 | 0.267 |
| A005 | 0.444 | 2166 | 2387 | 2663 | 221 | 497 | 0.444 | 0.444 |
| A007 | 0.259 | 1838 | 1931 | 2197 | 93 | 359 | 0.259 | 0.259 |
| A008 | 0.309 | 2160 | 2369 | 2837 | 209 | 677 | 0.309 | 0.309 |
| A009 | 0.367 | 2391 | 2598 | 2956 | 207 | 565 | 0.367 | 0.367 |
| A010 | 0.500 | 1716 | 1925 | 2134 | 209 | 418 | 0.500 | 0.500 |
| A011 | 0.326 | 1788 | 1935 | 2240 | 147 | 452 | 0.326 | 0.326 |
| A012 | 0.364 | 2233 | 2428 | 2769 | 195 | 536 | 0.364 | 0.364 |
| A013 | 0.338 | 2409 | 2667 | 3173 | 258 | 764 | 0.338 | 0.338 |
| A014 | 0.391 | 1701 | 1836 | 2046 | 135 | 345 | 0.391 | 0.391 |
| A015 | 0.400 | 1715 | 1877 | 2120 | 162 | 405 | 0.400 | 0.400 |
| A016 | 0.159 | 2233 | 2336 | 2880 | 103 | 647 | 0.159 | 0.159 |
| A017 | 0.333 | 1728 | 1882 | 2190 | 154 | 462 | 0.333 | 0.333 |
| A018 | 0.538 | 1862 | 2175 | 2443 | 313 | 581 | 0.538 | 0.538 |
| A019 | 0.333 | 1756 | 1927 | 2269 | 171 | 513 | 0.333 | 0.333 |
| A020 | 0.379 | 2535 | 2761 | 3131 | 226 | 596 | 0.379 | 0.379 |
| A021 | 0.212 | 2151 | 2283 | 2773 | 132 | 622 | 0.212 | 0.212 |
| A022 | 0.340 | 1900 | 2089 | 2456 | 189 | 556 | 0.340 | 0.340 |
| A023 | 0.232 | 2251 | 2384 | 2824 | 133 | 573 | 0.232 | 0.232 |
| A024 | 0.268 | 2522 | 2676 | 3097 | 154 | 575 | 0.268 | 0.268 |
| A025 | 0.267 | 1708 | 1775 | 1959 | 67 | 251 | 0.267 | 0.267 |
| A026 | 0.273 | 1611 | 1730 | 2047 | 119 | 436 | 0.273 | 0.273 |
| A027 | 0.340 | 1537 | 1689 | 1984 | 152 | 447 | 0.340 | 0.340 |
| A028 | 0.274 | 1780 | 1927 | 2316 | 147 | 536 | 0.274 | 0.274 |
| A029 | 0.323 | 1839 | 2023 | 2409 | 184 | 570 | 0.323 | 0.323 |
| A030 | 0.259 | 2051 | 2245 | 2799 | 194 | 748 | 0.259 | 0.259 |
| A031 | 0.393 | 2107 | 2321 | 2652 | 214 | 545 | 0.393 | 0.393 |
| A032 | 0.146 | 2584 | 2678 | 3226 | 94 | 642 | 0.146 | 0.146 |
| A033 | 0.298 | 2251 | 2426 | 2839 | 175 | 588 | 0.298 | 0.298 |
| A034 | 0.340 | 1909 | 2107 | 2491 | 198 | 582 | 0.340 | 0.340 |
| A035 | 0.333 | 3037 | 3305 | 3841 | 268 | 804 | 0.333 | 0.333 |
| A036 | 0.293 | 2211 | 2417 | 2915 | 206 | 704 | 0.293 | 0.293 |
| A037 | 0.326 | 2173 | 2335 | 2670 | 162 | 497 | 0.326 | 0.326 |
| A038 | 0.417 | 1543 | 1713 | 1951 | 170 | 408 | 0.417 | 0.417 |
| A039 | 0.391 | 1572 | 1721 | 1953 | 149 | 381 | 0.391 | 0.391 |
| A040 | 0.250 | 1959 | 2119 | 2599 | 160 | 640 | 0.250 | 0.250 |
| A041 | 0.219 | 1993 | 2144 | 2682 | 151 | 689 | 0.219 | 0.219 |
| A042 | 0.371 | 2660 | 2929 | 3384 | 269 | 724 | 0.371 | 0.371 |
| A044 | 0.350 | 2657 | 2858 | 3231 | 201 | 574 | 0.350 | 0.350 |
| A045 | 0.231 | 2175 | 2325 | 2825 | 150 | 650 | 0.231 | 0.231 |
| A047 | 0.396 | 2197 | 2458 | 2856 | 261 | 659 | 0.396 | 0.396 |
| A048 | 0.316 | 2535 | 2783 | 3320 | 248 | 785 | 0.316 | 0.316 |
| A049 | 0.400 | 2004 | 2256 | 2634 | 252 | 630 | 0.400 | 0.400 |
| A050 | 0.308 | 2193 | 2403 | 2876 | 210 | 683 | 0.308 | 0.308 |
| A051 | 0.239 | 1745 | 1867 | 2255 | 122 | 510 | 0.239 | 0.239 |
| A052 | 0.289 | 2073 | 2247 | 2674 | 174 | 601 | 0.289 | 0.289 |
| A053 | 0.250 | 2239 | 2353 | 2695 | 114 | 456 | 0.250 | 0.250 |
| A054 | 0.389 | 1816 | 2005 | 2302 | 189 | 486 | 0.389 | 0.389 |
| A055 | 0.374 | 3127 | 3668 | 4575 | 541 | 1448 | 0.374 | 0.374 |
| A056 | 0.244 | 2538 | 2728 | 3316 | 190 | 778 | 0.244 | 0.244 |
| A057 | 0.308 | 2125 | 2263 | 2574 | 138 | 449 | 0.308 | 0.308 |
| A058 | 0.268 | 4120 | 4529 | 5647 | 409 | 1527 | 0.268 | 0.268 |
| A059 | 0.385 | 2164 | 2358 | 2668 | 194 | 504 | 0.385 | 0.385 |
| A060 | 0.333 | 2325 | 2494 | 2832 | 169 | 507 | 0.333 | 0.333 |
| A061 | 0.300 | 2006 | 2205 | 2669 | 199 | 663 | 0.300 | 0.300 |
| A062 | 0.265 | 3718 | 4058 | 5002 | 340 | 1284 | 0.265 | 0.265 |
| A063 | 0.419 | 2231 | 2584 | 3073 | 353 | 842 | 0.419 | 0.419 |
| A064 | 0.333 | 1926 | 2076 | 2376 | 150 | 450 | 0.333 | 0.333 |
| A065 | 0.415 | 2225 | 2494 | 2874 | 269 | 649 | 0.415 | 0.415 |
| A066 | 0.500 | 1761 | 1968 | 2175 | 207 | 414 | 0.500 | 0.500 |
| A067 | 0.284 | 1701 | 1852 | 2233 | 151 | 532 | 0.284 | 0.284 |
| A068 | 0.350 | 1979 | 2215 | 2653 | 236 | 674 | 0.350 | 0.350 |
| A069 | 0.167 | 1935 | 1998 | 2313 | 63 | 378 | 0.167 | 0.167 |
| A070 | 0.244 | 1939 | 2063 | 2446 | 124 | 507 | 0.244 | 0.244 |
| A071 | 0.333 | 1762 | 1950 | 2326 | 188 | 564 | 0.333 | 0.333 |
| A072 | 0.299 | 1723 | 1912 | 2356 | 189 | 633 | 0.299 | 0.299 |
| A073 | 0.309 | 1614 | 1774 | 2132 | 160 | 518 | 0.309 | 0.309 |
| A074 | 0.441 | 1698 | 1884 | 2120 | 186 | 422 | 0.441 | 0.441 |
| A075 | 0.500 | 1489 | 1620 | 1751 | 131 | 262 | 0.500 | 0.500 |
| A076 | 0.278 | 1529 | 1684 | 2087 | 155 | 558 | 0.278 | 0.278 |
| A077 | 0.286 | 2845 | 3154 | 3927 | 309 | 1082 | 0.286 | 0.286 |
| A078 | 0.410 | 1867 | 2081 | 2389 | 214 | 522 | 0.410 | 0.410 |
| A080 | 0.185 | 3548 | 3924 | 5583 | 376 | 2035 | 0.185 | 0.185 |
| A081 | 0.190 | 2698 | 2853 | 3512 | 155 | 814 | 0.190 | 0.190 |
| A082 | 0.210 | 1625 | 1744 | 2193 | 119 | 568 | 0.210 | 0.210 |
| A083 | 0.294 | 1583 | 1692 | 1954 | 109 | 371 | 0.294 | 0.294 |
| A084 | 0.156 | 3394 | 3647 | 5013 | 253 | 1619 | 0.156 | 0.156 |
| A085 | 0.442 | 2416 | 2867 | 3436 | 451 | 1020 | 0.442 | 0.442 |
| A086 | 0.269 | 2111 | 2293 | 2788 | 182 | 677 | 0.269 | 0.269 |
| A087 | 0.344 | 1740 | 1924 | 2274 | 184 | 534 | 0.344 | 0.344 |
| A088 | 0.311 | 1715 | 1881 | 2249 | 166 | 534 | 0.311 | 0.311 |
| A089 | 0.250 | 1876 | 1981 | 2296 | 105 | 420 | 0.250 | 0.250 |
| A090 | 0.230 | 3411 | 3775 | 4993 | 364 | 1582 | 0.230 | 0.230 |
| A091 | 0.215 | 3897 | 4201 | 5308 | 304 | 1411 | 0.215 | 0.215 |
| A092 | 0.342 | 1906 | 2151 | 2622 | 245 | 716 | 0.342 | 0.342 |
| A093 | 0.378 | 2821 | 3197 | 3815 | 376 | 994 | 0.378 | 0.378 |
| A094 | 0.273 | 2447 | 2600 | 3008 | 153 | 561 | 0.273 | 0.273 |
| A095 | 0.292 | 1573 | 1726 | 2098 | 153 | 525 | 0.292 | 0.292 |
| A096 | 0.320 | 1784 | 1913 | 2187 | 129 | 403 | 0.320 | 0.320 |
| A097 | 0.276 | 1374 | 1479 | 1755 | 105 | 381 | 0.276 | 0.276 |
| A098 | 0.351 | 1480 | 1655 | 1979 | 175 | 499 | 0.351 | 0.351 |
| A099 | 0.229 | 1679 | 1770 | 2077 | 91 | 398 | 0.229 | 0.229 |
| A100 | 0.286 | 1538 | 1705 | 2123 | 167 | 585 | 0.286 | 0.286 |
| A101 | 0.324 | 2137 | 2344 | 2775 | 207 | 638 | 0.324 | 0.324 |
| A102 | 0.230 | 2473 | 2657 | 3275 | 184 | 802 | 0.230 | 0.230 |
| A103 | 0.284 | 1868 | 2069 | 2576 | 201 | 708 | 0.284 | 0.284 |
| A104 | 0.378 | 2344 | 2732 | 3369 | 388 | 1025 | 0.378 | 0.378 |
| A105 | 0.292 | 2427 | 2750 | 3532 | 323 | 1105 | 0.292 | 0.292 |
| A107 | 0.217 | 2140 | 2305 | 2902 | 165 | 762 | 0.217 | 0.217 |
| A108 | 0.364 | 1876 | 2034 | 2311 | 158 | 435 | 0.364 | 0.364 |
| A109 | 0.421 | 1900 | 2206 | 2627 | 306 | 727 | 0.421 | 0.421 |
| A110 | 0.310 | 2621 | 3048 | 4001 | 427 | 1380 | 0.310 | 0.310 |
| A111 | 0.393 | 2064 | 2409 | 2942 | 345 | 878 | 0.393 | 0.393 |
| A112 | 0.190 | 2000 | 2165 | 2868 | 165 | 868 | 0.190 | 0.190 |
| A113 | 0.283 | 1699 | 1872 | 2310 | 173 | 611 | 0.283 | 0.283 |
| A114 | 0.429 | 1838 | 2101 | 2452 | 263 | 614 | 0.429 | 0.429 |
| A115 | 0.297 | 2091 | 2281 | 2731 | 190 | 640 | 0.297 | 0.297 |
| A116 | 0.321 | 1571 | 1707 | 1994 | 136 | 423 | 0.321 | 0.321 |
| A117 | 0.275 | 1691 | 1874 | 2356 | 183 | 665 | 0.275 | 0.275 |
| A118 | 0.267 | 1835 | 1969 | 2338 | 134 | 503 | 0.267 | 0.267 |
| A119 | 0.260 | 2118 | 2320 | 2895 | 202 | 777 | 0.260 | 0.260 |
| A120 | 0.265 | 1833 | 1960 | 2313 | 127 | 480 | 0.265 | 0.265 |
| A121 | 0.308 | 1825 | 1992 | 2368 | 167 | 543 | 0.308 | 0.308 |
| A122 | 0.397 | 1674 | 1931 | 2322 | 257 | 648 | 0.397 | 0.397 |
| A123 | 0.320 | 1669 | 1824 | 2153 | 155 | 484 | 0.320 | 0.320 |
| A124 | 0.360 | 1627 | 1766 | 2013 | 139 | 386 | 0.360 | 0.360 |
| A125 | 0.181 | 1485 | 1591 | 2072 | 106 | 587 | 0.181 | 0.181 |
| A126 | 0.591 | 2476 | 2969 | 3310 | 493 | 834 | 0.591 | 0.591 |
| A127 | 0.160 | 1935 | 2040 | 2591 | 105 | 656 | 0.160 | 0.160 |
| A128 | 0.220 | 2485 | 2627 | 3132 | 142 | 647 | 0.220 | 0.220 |
| A129 | 0.255 | 3083 | 3385 | 4268 | 302 | 1185 | 0.255 | 0.255 |
| A130 | 0.254 | 3137 | 3330 | 3896 | 193 | 759 | 0.254 | 0.254 |
| A131 | 0.364 | 1729 | 1930 | 2282 | 201 | 553 | 0.364 | 0.364 |
| A132 | 0.364 | 2288 | 2601 | 3149 | 313 | 861 | 0.364 | 0.364 |
| A133 | 0.400 | 2132 | 2531 | 3130 | 399 | 998 | 0.400 | 0.400 |
| A134 | 0.500 | 3654 | 4285 | 4916 | 631 | 1262 | 0.500 | 0.500 |
| A135 | 0.341 | 1511 | 1652 | 1924 | 141 | 413 | 0.341 | 0.341 |
| A136 | 0.197 | 2697 | 2874 | 3596 | 177 | 899 | 0.197 | 0.197 |
| A137 | 0.420 | 1797 | 1980 | 2233 | 183 | 436 | 0.420 | 0.420 |
| A138 | 0.315 | 1931 | 2137 | 2585 | 206 | 654 | 0.315 | 0.315 |
| A139 | 0.227 | 1905 | 2069 | 2629 | 164 | 724 | 0.227 | 0.227 |
| A140 | 0.294 | 1483 | 1623 | 1959 | 140 | 476 | 0.294 | 0.294 |
| A141 | 0.315 | 1872 | 2044 | 2418 | 172 | 546 | 0.315 | 0.315 |
| A142 | 0.435 | 2390 | 2573 | 2811 | 183 | 421 | 0.435 | 0.435 |
| A143 | 0.515 | 2047 | 2421 | 2773 | 374 | 726 | 0.515 | 0.515 |
| A144 | 0.235 | 2017 | 2143 | 2553 | 126 | 536 | 0.235 | 0.235 |
| A145 | 0.246 | 1492 | 1602 | 1939 | 110 | 447 | 0.246 | 0.246 |
| A146 | 0.222 | 1899 | 2068 | 2660 | 169 | 761 | 0.222 | 0.222 |
| A147 | 0.288 | 1608 | 1738 | 2059 | 130 | 451 | 0.288 | 0.288 |
| A148 | 0.191 | 1967 | 2090 | 2610 | 123 | 643 | 0.191 | 0.191 |
| A149 | 0.328 | 1581 | 1718 | 1999 | 137 | 418 | 0.328 | 0.328 |
| A150 | 0.247 | 1558 | 1690 | 2092 | 132 | 534 | 0.247 | 0.247 |
| A151 | 0.289 | 2177 | 2402 | 2954 | 225 | 777 | 0.289 | 0.289 |
| A152 | 0.324 | 1876 | 2006 | 2278 | 130 | 402 | 0.324 | 0.324 |
| A153 | 0.232 | 1713 | 1859 | 2343 | 146 | 630 | 0.232 | 0.232 |
| A154 | 0.262 | 1887 | 2053 | 2520 | 166 | 633 | 0.262 | 0.262 |
| A155 | 0.368 | 2906 | 3327 | 4049 | 421 | 1143 | 0.368 | 0.368 |
| A156 | 0.207 | 2191 | 2291 | 2674 | 100 | 483 | 0.207 | 0.207 |
| A157 | 0.385 | 1886 | 2065 | 2351 | 179 | 465 | 0.385 | 0.385 |
| A158 | 0.235 | 2424 | 2551 | 2964 | 127 | 540 | 0.235 | 0.235 |
| A159 | 0.290 | 2678 | 2973 | 3694 | 295 | 1016 | 0.290 | 0.290 |
| A160 | 0.435 | 2160 | 2489 | 2915 | 329 | 755 | 0.435 | 0.435 |
| A161 | 0.159 | 1674 | 1762 | 2227 | 88 | 553 | 0.159 | 0.159 |
| A162 | 0.232 | 3480 | 3835 | 5011 | 355 | 1531 | 0.232 | 0.232 |
| A163 | 0.238 | 2505 | 2697 | 3311 | 192 | 806 | 0.238 | 0.238 |
| A164 | 0.250 | 2535 | 2718 | 3267 | 183 | 732 | 0.250 | 0.250 |
| A165 | 0.197 | 2072 | 2189 | 2665 | 117 | 593 | 0.197 | 0.197 |
| A166 | 0.288 | 1883 | 2051 | 2467 | 168 | 584 | 0.288 | 0.288 |
| A167 | 0.214 | 2228 | 2321 | 2662 | 93 | 434 | 0.214 | 0.214 |
| A168 | 0.415 | 2366 | 2847 | 3525 | 481 | 1159 | 0.415 | 0.415 |
| A169 | 0.136 | 2543 | 2661 | 3412 | 118 | 869 | 0.136 | 0.136 |
| A170 | 0.351 | 1456 | 1589 | 1835 | 133 | 379 | 0.351 | 0.351 |
| A171 | 0.400 | 2463 | 2761 | 3208 | 298 | 745 | 0.400 | 0.400 |
| A172 | 0.200 | 1944 | 2070 | 2574 | 126 | 630 | 0.200 | 0.200 |
| A173 | 0.263 | 1505 | 1600 | 1866 | 95 | 361 | 0.263 | 0.263 |
| A174 | 0.254 | 1687 | 1816 | 2194 | 129 | 507 | 0.254 | 0.254 |
| A175 | 0.383 | 1681 | 1821 | 2047 | 140 | 366 | 0.383 | 0.383 |
| A176 | 0.245 | 2344 | 2544 | 3159 | 200 | 815 | 0.245 | 0.245 |
| A177 | 0.344 | 1596 | 1733 | 1995 | 137 | 399 | 0.344 | 0.344 |
| A178 | 0.262 | 2019 | 2183 | 2644 | 164 | 625 | 0.262 | 0.262 |
| A179 | 0.222 | 2056 | 2181 | 2619 | 125 | 563 | 0.222 | 0.222 |
| A180 | 0.375 | 1891 | 2096 | 2438 | 205 | 547 | 0.375 | 0.375 |
| A181 | 0.413 | 2575 | 2959 | 3505 | 384 | 930 | 0.413 | 0.413 |
| A182 | 0.204 | 1828 | 1930 | 2328 | 102 | 500 | 0.204 | 0.204 |
| A183 | 0.364 | 1523 | 1644 | 1856 | 121 | 333 | 0.364 | 0.364 |
| A184 | 0.214 | 2049 | 2187 | 2693 | 138 | 644 | 0.214 | 0.214 |
| A185 | 0.236 | 2417 | 2606 | 3217 | 189 | 800 | 0.236 | 0.236 |
| A186 | 0.455 | 2223 | 2909 | 3729 | 686 | 1506 | 0.455 | 0.455 |
| A187 | 0.234 | 1654 | 1755 | 2086 | 101 | 432 | 0.234 | 0.234 |
| A188 | 0.311 | 2229 | 2460 | 2972 | 231 | 743 | 0.311 | 0.311 |
| A189 | 0.392 | 2320 | 2588 | 3003 | 268 | 683 | 0.392 | 0.392 |
| A190 | 0.300 | 2473 | 2670 | 3130 | 197 | 657 | 0.300 | 0.300 |
| A191 | 0.276 | 1782 | 1907 | 2235 | 125 | 453 | 0.276 | 0.276 |
| A192 | 0.258 | 2127 | 2255 | 2623 | 128 | 496 | 0.258 | 0.258 |
| A193 | 0.214 | 1788 | 1920 | 2404 | 132 | 616 | 0.214 | 0.214 |
| A194 | 0.313 | 1930 | 2107 | 2496 | 177 | 566 | 0.313 | 0.313 |
| A195 | 0.235 | 1581 | 1710 | 2129 | 129 | 548 | 0.235 | 0.235 |
| A196 | 0.244 | 1821 | 1958 | 2381 | 137 | 560 | 0.244 | 0.244 |
| A197 | 0.203 | 1743 | 1835 | 2196 | 92 | 453 | 0.203 | 0.203 |
| A198 | 0.400 | 1696 | 1912 | 2236 | 216 | 540 | 0.400 | 0.400 |
| A199 | 0.417 | 1498 | 1665 | 1899 | 167 | 401 | 0.417 | 0.417 |
| A200 | 0.281 | 1441 | 1554 | 1843 | 113 | 402 | 0.281 | 0.281 |
| A201 | 0.273 | 2036 | 2205 | 2656 | 169 | 620 | 0.273 | 0.273 |
| A202 | 0.345 | 1898 | 2080 | 2426 | 182 | 528 | 0.345 | 0.345 |
| A203 | 0.244 | 1768 | 1880 | 2226 | 112 | 458 | 0.244 | 0.244 |
| A204 | 0.385 | 1642 | 1820 | 2105 | 178 | 463 | 0.385 | 0.385 |
| A205 | 0.256 | 1851 | 1983 | 2367 | 132 | 516 | 0.256 | 0.256 |
| A207 | 0.190 | 2173 | 2299 | 2835 | 126 | 662 | 0.190 | 0.190 |
| A208 | 0.333 | 2277 | 2531 | 3039 | 254 | 762 | 0.333 | 0.333 |
| A209 | 0.372 | 1721 | 1937 | 2302 | 216 | 581 | 0.372 | 0.372 |
| A210 | 0.278 | 1907 | 2066 | 2479 | 159 | 572 | 0.278 | 0.278 |
| A211 | 0.240 | 2153 | 2306 | 2791 | 153 | 638 | 0.240 | 0.240 |
| A212 | 0.421 | 2143 | 2458 | 2891 | 315 | 748 | 0.421 | 0.421 |
| A213 | 0.264 | 2057 | 2332 | 3099 | 275 | 1042 | 0.264 | 0.264 |
| A214 | 0.300 | 2116 | 2363 | 2939 | 247 | 823 | 0.300 | 0.300 |
| A215 | 0.255 | 1982 | 2118 | 2515 | 136 | 533 | 0.255 | 0.255 |
| A216 | 0.273 | 2799 | 3061 | 3760 | 262 | 961 | 0.273 | 0.273 |
| A217 | 0.244 | 2021 | 2237 | 2906 | 216 | 885 | 0.244 | 0.244 |
| A218 | 0.293 | 2319 | 2571 | 3179 | 252 | 860 | 0.293 | 0.293 |
| A219 | 0.393 | 2098 | 2309 | 2635 | 211 | 537 | 0.393 | 0.393 |
| A220 | 0.302 | 1803 | 1943 | 2266 | 140 | 463 | 0.302 | 0.302 |
| A222 | 0.310 | 1705 | 1876 | 2256 | 171 | 551 | 0.310 | 0.310 |
| A223 | 0.310 | 1593 | 1732 | 2041 | 139 | 448 | 0.310 | 0.310 |
| A224 | 0.357 | 1649 | 1811 | 2103 | 162 | 454 | 0.357 | 0.357 |
| A225 | 0.368 | 1655 | 1824 | 2114 | 169 | 459 | 0.368 | 0.368 |

Comparative Results of nATFt's and nATFz's

Results between patients in two different geographic locations (i.e., two different hospitals) were compared for correlation with each other. This comparison is expressed in Table 4 below, and includes a comparison of INR values calculated by the WHO method for each respective location, with GInr representing one location for these traditionally WHO determined values, and MInr representing values based on data obtained at the other location. The values identified as ATFz and ATFt, such as, GATFt and MATFt, and GATFz and MATFz, represent anticoagulant therapy factors derived from the expressions (1) through (9) above.

The ATFa represents an anticoagulation therapy factor derived from our method and apparatus for the expression ATFa=XR^{(2-nFTR)} wherein a maximum acceleration point is obtained, and nFTR=IUX/IUT, where IUX is the change in optical density from a time prior to the MAP time (t_{<MAP} which is t_{MAP} minus some time from MAP) to the optical density at a time after the MAP time (t_{>MAP} which is t_{MAP} plus some time from MAP); and wherein IUT=the change in optical density at the time t₁ to the optical density measured at time t_{EOT}, where time t_{EOT} is the end of the test (EOT). The (IUX) represents the fibrinogen (FBG) for MAP (−a number of seconds) to MAP (+a number of seconds) (that is the fibrinogen (FBG) converted from t_{<MAP} to t_{>MAP} on FIG. 2) The (IUT) represents fibrinogen converted from c₁ to c_{EOT} (that is the fibrinogen converted from t₁ to t_{EOT}, see FIG. 2). The XR for the ATFa expression is XR=TX/MNTX, which is the ratio of time to map (TX) by the mean normal time to map of 20 presumed “normal” patients.

TABLE 4
COMPARATIVE RESULTS FOR ATFt and ATFz

| | | | | | Std. | | |
|---|---|---|---|---|---|---|---|
| Comparison | n | r | m | b | Error | Ng | Lassen |
| GInr vs. | 129 | 0.996 | 0.891 | 0.148 | 0.082 | 6/129 = | delta <= 0.4 5@96.1% |
| GATFa | | | | | | 4.7% | delta <= 0.7 2@98.4% |
| | | | | | | mismatches | |
| GInr vs. | 129 | 0.975 | 1.014 | −0.016 | 0.215 | 15/129 = | delta <= 0.4 9@93% |
| GATFz | | | | | | 11.6% | delta <= 0.7 3@97.7% |
| | | | | | | mismatches | |
| GInr vs. | 129 | 0.971 | 0.895 | 0.332 | 0.232 | 26/129 = | delta <= 0.4 18@86.0% |
| GATFt | | | | | | 20.2% | delta <= 0.7 2@98.4% |
| | | | | | | mismatches | |
| MInr vs. | 129 | 0.996 | 0.943 | 0.082 | 0.094 | 18/129 = | delta <= 0.4 15@88.4% |
| MATFa | | | | | | 14.0% | delta <= 0.7 5@96.1% |
| | | | | | | mismatches | |
| MInr vs. | 129 | 0.985 | 0.993 | −0.058 | 0.177 | 2/129 = | delta <= 0.4 0@100% |
| MATFz | | | | | | 1.6% | delta <= 0.7 0@100% |
| | | | | | | mismatches | |
| MInr vs. | 129 | 0.981 | 0.851 | 0.420 | 0.200 | 8/129 = | delta <= 0.4 6@95.3 |
| MATFt | | | | | | 6.2% | delta <= 0.7 2@98.4% |
| | | | | | | mismatches | |

A comparison of combined location data is shown in Table 5, below. The sample size was 217.

TABLE 5
STATISTICAL SUMMARY OF MHTL DATA

| Com- | | | | | Std. | | |
|---|---|---|---|---|---|---|---|
| parison | n | r | m | b | Error | Ng | Lassen |
| Inr vs | 217 | 0.984 | 1.006 | 0.011 | 0.215 | 30/217 = | delta <= 0.4 |
| ATFa | | | | | | 13.8% | 16@92.6% |
| | | | | | | mismatches | delta <= 0.7 |
| | | | | | | | 1@99.5% |
| Inr vs. | 217 | 0.984 | 1.002 | 0.120 | 0.214 | 26/217 = | delta <= 0.4 |
| ATFz | | | | | | 12.0% | 18@91.7% |
| | | | | | | mismatched | delta <= 0.7 |
| | | | | | | | 3@98.6% |
| Inr vs. | 217 | 0.984 | 0.900 | 0.482 | 1.218 | 45/217 = | delta <= 0.4 |
| ATFt | | | | | | 20.7% | 43@80.2% |
| | | | | | | mismatches | delta <= 0.7 |
| | | | | | | | 6@97.2% |

Comparative results were also calculated for the ATFt which includes the lag phase fibrinogen, in accordance with the IULz, using the expression (5.1) for the TEOT value. Table 6 below provides the values for the ATFz, ATFt, and the ATFt2 (which is obtained from expression 5.1 using the IULz).

TABLE 6

| ID | INR | INRz | ATFt | ATFt2 |
|---|---|---|---|---|
| A001 | 3.1 | 2.9 | 2.4 | 2.6 |
| A002 | 3.3 | 2.9 | 2.4 | 2.6 |
| A003 | 3.3 | 2.9 | 2.4 | 2.6 |
| A004 | 2.1 | 2.3 | 1.8 | 2.0 |
| A005 | 2.9 | 2.6 | 2.1 | 2.3 |
| A007 | 2.1 | 2.0 | 1.5 | 1.6 |
| A008 | 2.8 | 2.8 | 2.3 | 2.5 |
| A009 | 3.4 | 3.1 | 2.6 | 2.8 |
| A010 | 1.9 | 1.8 | 1.3 | 1.5 |
| A011 | 2.1 | 1.9 | 1.5 | 1.6 |
| A012 | 3.2 | 2.8 | 2.3 | 2.5 |
| A013 | 3.5 | 3.3 | 2.8 | 3.0 |
| A014 | 1.8 | 1.7 | 1.3 | 1.4 |
| A015 | 1.9 | 1.8 | 1.3 | 1.5 |
| A016 | 3.2 | 2.9 | 2.4 | 2.6 |
| A017 | 1.8 | 1.9 | 1.4 | 1.6 |
| A018 | 2.2 | 2.1 | 1.7 | 1.8 |
| A019 | 1.8 | 1.9 | 1.5 | 1.6 |
| A020 | 3.5 | 3.4 | 2.9 | 3.2 |
| A021 | 2.8 | 2.7 | 2.2 | 2.4 |
| A022 | 2.2 | 2.2 | 1.7 | 1.9 |
| A023 | 3.2 | 2.9 | 2.3 | 2.5 |
| A024 | 3.7 | 3.5 | 2.9 | 3.1 |
| A025 | 1.8 | 1.7 | 1.2 | 1.4 |
| A026 | 1.6 | 1.6 | 1.2 | 1.4 |
| A027 | 1.5 | 1.5 | 1.1 | 1.3 |
| A028 | 1.9 | 2.0 | 1.5 | 1.7 |
| A029 | 2.1 | 2.1 | 1.6 | 1.8 |
| A030 | 2.6 | 2.6 | 2.1 | 2.3 |
| A031 | 2.7 | 2.5 | 2.1 | 2.3 |
| A032 | 4.1 | 3.8 | 3.1 | 3.3 |
| A033 | 2.9 | 2.9 | 2.4 | 2.6 |
| A034 | 2.2 | 2.2 | 1.7 | 1.9 |
| A035 | 4.9 | 4.7 | 4.3 | 4.7 |
| A036 | 3.2 | 2.9 | 2.4 | 2.6 |
| A037 | 2.5 | 2.7 | 2.1 | 2.4 |
| A038 | 1.6 | 1.6 | 1.1 | 1.2 |
| A039 | 1.4 | 1.6 | 1.1 | 1.3 |
| A040 | 2.4 | 2.4 | 1.9 | 2.1 |
| A041 | 2.3 | 2.4 | 2.0 | 2.2 |
| A042 | 4.1 | 3.8 | 3.3 | 3.6 |
| A044 | 4.2 | 3.7 | 3.2 | 3.4 |
| A045 | 2.7 | 2.8 | 2.3 | 2.5 |
| A047 | 2.8 | 2.8 | 2.3 | 2.5 |
| A048 | 3.9 | 3.6 | 3.1 | 3.3 |
| A049 | 2.6 | 2.4 | 1.9 | 2.1 |
| A050 | 2.8 | 2.8 | 2.3 | 2.5 |
| A051 | 1.9 | 1.9 | 1.4 | 1.6 |
| A052 | 2.8 | 2.6 | 2.0 | 2.2 |
| A053 | 3.0 | 2.8 | 2.2 | 2.4 |
| A054 | 2.1 | 2.0 | 1.5 | 1.7 |
| A055 | 5.6 | 5.4 | 5.3 | 5.6 |
| A056 | 3.6 | 3.7 | 3.1 | 3.4 |
| A057 | 2.8 | 2.6 | 2.0 | 2.2 |
| A058 | 8.5 | 8.7 | 8.6 | 9.1 |
| A059 | 2.9 | 2.6 | 2.1 | 2.3 |
| A060 | 3.5 | 3.0 | 2.4 | 2.6 |
| A061 | 2.4 | 2.5 | 2.0 | 2.1 |
| A062 | 7.0 | 7.2 | 6.8 | 7.3 |
| A063 | 3.0 | 3.0 | 2.5 | 2.7 |
| A064 | 2.2 | 2.2 | 1.7 | 1.9 |
| A065 | 2.6 | 2.8 | 2.4 | 2.6 |
| A066 | 2.0 | 1.9 | 1.4 | 1.6 |
| A067 | 1.8 | 1.8 | 1.4 | 1.6 |
| A068 | 2.6 | 2.4 | 1.9 | 2.1 |
| A069 | 2.4 | 2.2 | 1.6 | 1.8 |
| A070 | 2.4 | 2.3 | 1.7 | 1.9 |
| A071 | 1.9 | 2.0 | 1.5 | 1.7 |
| A072 | 1.8 | 1.9 | 1.5 | 1.6 |
| A073 | 1.5 | 1.7 | 1.3 | 1.4 |
| A074 | 1.7 | 1.8 | 1.3 | 1.5 |
| A075 | 1.6 | 1.4 | 1.0 | 1.1 |
| A076 | 1.4 | 1.6 | 1.2 | 1.3 |
| A077 | 4.5 | 4.6 | 4.1 | 4.4 |
| A078 | 2.2 | 2.1 | 1.6 | 1.8 |
| A080 | 7.3 | 7.4 | 7.3 | 7.6 |
| A081 | 3.8 | 4.2 | 3.5 | 3.8 |
| A082 | 1.6 | 1.7 | 1.3 | 1.5 |
| A083 | 1.6 | 1.6 | 1.1 | 1.3 |
| A084 | 6.7 | 6.7 | 6.3 | 6.6 |
| A085 | 3.3 | 3.4 | 3.1 | 3.3 |
| A086 | 2.8 | 2.7 | 2.2 | 2.4 |
| A087 | 1.8 | 1.9 | 1.5 | 1.6 |
| A088 | 1.7 | 1.9 | 1.4 | 1.6 |
| A089 | 2.3 | 2.1 | 1.6 | 1.7 |
| A090 | 6.3 | 6.6 | 6.3 | 6.7 |
| A091 | 7.6 | 8.1 | 7.6 | 8.1 |
| A092 | 1.9 | 2.3 | 1.8 | 2.0 |
| A093 | 4.9 | 4.3 | 4.0 | 4.2 |
| A094 | 3.2 | 3.3 | 2.7 | 2.9 |
| A095 | 1.5 | 1.6 | 1.2 | 1.4 |
| A096 | 2.3 | 1.9 | 1.4 | 1.6 |
| A097 | 1.3 | 1.3 | 0.9 | 1.0 |
| A098 | 1.4 | 1.5 | 1.1 | 1.2 |
| A099 | 1.8 | 1.7 | 1.3 | 1.4 |
| A100 | 1.4 | 1.6 | 1.2 | 1.3 |
| A101 | 2.7 | 2.7 | 2.2 | 2.4 |
| A102 | 3.8 | 3.6 | 3.0 | 3.2 |
| A103 | 2.0 | 2.2 | 1.8 | 1.9 |
| A104 | 3.2 | 3.3 | 2.9 | 3.2 |
| A105 | 3.7 | 3.6 | 3.2 | 3.4 |
| A107 | 2.9 | 2.8 | 2.3 | 2.5 |
| A108 | 2.1 | 2.1 | 1.6 | 1.8 |
| A109 | 2.2 | 2.3 | 1.8 | 2.0 |
| A110 | 3.9 | 4.2 | 3.9 | 4.1 |
| A111 | 2.5 | 2.7 | 2.2 | 2.4 |
| A112 | 2.5 | 2.5 | 2.1 | 2.3 |
| A113 | 1.9 | 1.9 | 1.4 | 1.6 |
| A114 | 2.1 | 2.1 | 1.7 | 1.8 |
| A115 | 2.4 | 2.6 | 2.1 | 2.3 |
| A116 | 1.7 | 1.6 | 1.2 | 1.3 |
| A117 | 1.6 | 1.9 | 1.5 | 1.6 |
| A118 | 2.1 | 2.1 | 1.6 | 1.7 |
| A119 | 3.0 | 2.7 | 2.3 | 2.4 |
| A120 | 2.1 | 2.0 | 1.6 | 1.7 |
| A121 | 2.2 | 2.1 | 1.6 | 1.7 |
| A122 | 1.7 | 1.9 | 1.4 | 1.6 |
| A123 | 1.8 | 1.8 | 1.3 | 1.5 |
| A124 | 1.8 | 1.7 | 1.2 | 1.3 |
| A125 | 1.4 | 1.4 | 1.1 | 1.3 |
| A126 | 3.7 | 3.2 | 3.0 | 3.3 |
| A127 | 2.4 | 2.3 | 1.8 | 2.0 |
| A128 | 3.8 | 3.5 | 2.9 | 3.1 |
| A129 | 5.3 | 5.3 | 4.8 | 5.3 |
| A130 | 4.7 | 5.2 | 4.5 | 4.9 |
| A131 | 1.7 | 1.9 | 1.5 | 1.6 |
| A132 | 2.8 | 3.1 | 2.7 | 2.9 |
| A133 | 2.6 | 2.9 | 2.5 | 2.7 |
| A134 | 6.6 | 6.0 | 6.6 | 7.1 |
| A135 | 1.5 | 1.5 | 1.1 | 1.2 |
| A136 | 4.3 | 4.2 | 3.6 | 3.8 |
| A137 | 1.9 | 1.9 | 1.5 | 1.6 |
| A138 | 2.0 | 2.3 | 1.8 | 2.0 |
| A139 | 2.1 | 2.3 | 1.8 | 2.0 |
| A140 | 1.3 | 1.5 | 1.1 | 1.2 |
| A141 | 2.2 | 2.1 | 1.7 | 1.8 |
| A142 | 3.4 | 2.9 | 2.5 | 2.7 |
| A143 | 2.5 | 2.5 | 2.1 | 2.3 |
| A144 | 2.5 | 2.4 | 1.9 | 2.1 |
| A145 | 1.4 | 1.4 | 1.1 | 1.2 |
| A146 | 2.3 | 2.3 | 1.9 | 2.0 |
| A147 | 1.7 | 1.6 | 1.2 | 1.4 |
| A148 | 2.3 | 2.4 | 1.9 | 2.1 |
| A149 | 1.6 | 1.6 | 1.2 | 1.3 |
| A150 | 1.6 | 1.6 | 1.2 | 1.3 |
| A151 | 2.8 | 2.9 | 2.4 | 2.6 |
| A152 | 2.2 | 2.1 | 1.6 | 4.7 |
| A153 | 1.8 | 1.9 | 1.5 | 1.6 |
| A154 | 2.2 | 2.2 | 1.7 | 1.9 |
| A155 | 4.8 | 4.6 | 4.3 | 4.7 |
| A156 | 2.9 | 2.8 | 2.2 | 2.4 |
| A157 | 2.1 | 2.1 | 1.6 | 1.8 |
| A158 | 3.6 | 3.3 | 2.6 | 2.8 |
| A159 | 3.9 | 4.1 | 3.6 | 3.9 |
| A160 | 2.7 | 2.8 | 2.3 | 2.5 |
| A161 | 1.7 | 1.8 | 1.4 | 1.5 |
| A162 | 6.6 | 6.8 | 6.4 | 6.9 |
| A163 | 3.9 | 3.6 | 3.1 | 3.3 |
| A164 | 4.0 | 3.6 | 3.0 | 3.3 |
| A165 | 2.7 | 2.6 | 2.0 | 2.2 |
| A166 | 2.2 | 2.2 | 1.7 | 1.9 |
| A167 | 2.9 | 2.8 | 2.2 | 2.4 |
| A168 | 3.6 | 3.5 | 3.1 | 3.3 |
| A169 | 4.1 | 3.8 | 3.2 | 3.4 |
| A170 | 1.4 | 1.4 | 1.0 | 1.1 |
| A171 | 3.4 | 3.3 | 2.9 | 3.1 |
| A172 | 2.5 | 2.3 | 1.8 | 2.0 |
| A173 | 1.6 | 1.4 | 1.0 | 1.1 |
| A174 | 1.8 | 1.8 | 1.4 | 1.5 |
| A175 | 1.8 | 1.7 | 1.3 | 1.4 |
| A176 | 3.4 | 3.3 | 2.7 | 2.9 |
| A177 | 1.7 | 1.6 | 1.2 | 1.3 |
| A178 | 2.3 | 2.5 | 2.0 | 2.1 |
| A179 | 2.6 | 2.5 | 2.0 | 2.2 |
| A180 | 2.3 | 2.2 | 1.7 | 1.9 |
| A181 | 3.5 | 3.7 | 3.3 | 3.6 |
| A182 | 2.1 | 2.0 | 1.6 | 1.7 |
| A183 | 1.5 | 1.5 | 1.0 | 1.2 |
| A184 | 2.6 | 2.5 | 2.0 | 2.2 |
| A185 | 3.3 | 3.4 | 2.9 | 3.1 |
| A186 | 3.1 | 3.5 | 3.1 | 3.3 |
| A187 | 1.8 | 1.7 | 1.3 | 1.4 |
| A188 | 3.1 | 2.9 | 2.4 | 2.6 |
| A189 | 3.0 | 3.0 | 2.6 | 2.8 |
| A190 | 3.6 | 3.4 | 2.8 | 3.1 |
| A191 | 2.0 | 1.9 | 1.5 | 1.6 |
| A192 | 2.7 | 2.6 | 2.1 | 2.3 |
| A193 | 2.1 | 2.0 | 1.6 | 1.7 |
| A194 | 2.2 | 2.3 | 1.8 | 2.0 |
| A195 | 1.4 | 1.6 | 1.2 | 1.4 |
| A196 | 2.0 | 2.1 | 1.6 | 1.8 |
| A197 | 1.8 | 1.9 | 1.4 | 1.5 |
| A198 | 2.0 | 1.9 | 1.4 | 1.5 |
| A199 | 1.5 | 1.5 | 1.0 | 1.2 |
| A200 | 1.4 | 1.4 | 1.0 | 1.1 |
| A201 | 2.6 | 2.5 | 2.0 | 2.2 |
| A202 | 2.5 | 2.2 | 1.7 | 1.9 |
| A203 | 2.0 | 1.9 | 1.4 | 1.6 |
| A204 | 1.8 | 1.7 | 1.3 | 1.4 |
| A205 | 1.9 | 2.1 | 1.6 | 1.8 |
| A207 | 2.7 | 2.8 | 2.3 | 2.5 |
| A208 | 3.0 | 3.0 | 2.5 | 2.8 |
| A209 | 1.9 | 1.9 | 1.5 | 1.6 |
| A210 | 2.4 | 2.2 | 1.7 | 1.9 |
| A211 | 2.9 | 2.7 | 2.2 | 2.4 |
| A212 | 2.8 | 2.7 | 2.3 | 2.5 |
| A213 | 2.7 | 2.8 | 2.3 | 2.5 |
| A214 | 2.8 | 2.8 | 2.3 | 2.5 |
| A215 | 2.5 | 2.3 | 1.8 | 2.0 |
| A216 | 4.1 | 4.4 | 3.9 | 4.2 |
| A217 | 2.3 | 2.6 | 2.2 | 2.3 |
| A218 | 2.9 | 3.2 | 2.7 | 3.0 |
| A219 | 2.7 | 2.5 | 2.0 | 2.2 |
| A220 | 2.0 | 2.0 | 1.5 | 1.7 |
| A222 | 2.0 | 1.9 | 1.4 | 1.6 |
| A223 | 1.7 | 1.6 | 1.2 | 1.4 |
| A224 | 1.6 | 1.7 | 1.3 | 1.4 |
| A225 | 1.8 | 1.7 | 1.3 | 1.4 |

Table 7 represents a comparison of the data from Table 6.

TABLE 7

| | | “r” | “m” | “b” | StdErr | StdDev |
|---|---|---|---|---|---|---|
| INR | INRz | 0.988 | 0.988 | 0.059 | 0.190 | 1.201 |
| vs | ATFt | 0.984 | 0.966 | 0.568 | 0.215 | 1.238 |
| | ATFt2 | 0.983 | 0.913 | 0.504 | 0.219 | 1.257 |
| ATFt vs | ATFt2 | 1.000 | 0.946 | −0.068 | 0.022 | 1.264 |

Table 8 provides comparative data for the anticoagulant therapy factors, similar to Table 2, but using the ATFt2 method from expressions (4) and (5.1) for corresponding GINRt2 and MINRt2 values.

TABLE 8

| ID | AINR | GINR | GINRa | GINRz | GINRt2 | MINR | MINRa | MINRz | MINRt2 |
|---|---|---|---|---|---|---|---|---|---|
| U0800 | 2.0 | 2.0 | 2.0 | 2.0 | 1.7 | 2.1 | 2.1 | 2.2 | 2.1 |
| U7440 | 2.6 | 3.0 | 3.0 | 2.9 | 2.9 | 3.0 | 3.0 | 2.8 | 3.4 |
| U7443 | 2.0 | 2.0 | 2.0 | 2.0 | 1.8 | 2.1 | 2.2 | 2.1 | 1.8 |
| U7458 | 1.4 | 1.4 | 1.4 | 1.4 | 1.2 | 1.4 | 1.4 | 1.3 | 1.3 |
| U7465 | 9.7 | 7.4 | 8.1 | 6.6 | 7.9 | 7.1 | 7.5 | 8.1 | 7.8 |
| U7469 | 1.1 | 1.1 | 1.1 | 1.1 | 0.9 | 1.2 | 1.1 | 1.1 | 1.0 |
| U7470 | 3.2 | 3.4 | 3.6 | 3.4 | 3.2 | 3.6 | 3.7 | 3.8 | 3.8 |
| U8080 | 3.1 | 3.6 | 3.6 | 3.3 | 3.6 | 3.3 | 3.3 | 3.5 | 3.4 |
| U8087 | 1.9 | 1.9 | 1.9 | 1.8 | 1.6 | 1.9 | 1.9 | 1.9 | 1.7 |
| U8092 | 1.7 | 1.7 | 1.8 | 1.7 | 1.6 | 1.9 | 1.9 | 1.9 | 1.6 |
| U3050 | 2.7 | 2.8 | 3.1 | 2.6 | 2.2 | 2.3 | 2.3 | 2.3 | 2.0 |
| U3077 | 1.3 | 1.4 | 1.4 | 1.4 | 1.1 | 1.3 | 1.3 | 1.3 | 1.2 |
| U3083 | 1.6 | 1.6 | 1.6 | 1.6 | 1.3 | 1.6 | 1.7 | 1.6 | 1.4 |
| U8210 | 2.6 | 2.9 | 3.0 | 2.8 | 2.7 | 2.7 | 2.8 | 2.8 | 2.6 |
| U8221 | 3.2 | 3.7 | 4.0 | 3.7 | 3.4 | 3.5 | 3.5 | 3.3 | 3.6 |
| U3408 | 1.1 | 1.2 | 1.2 | 1.2 | 0.9 | 1.1 | 1.0 | 1.0 | 0.9 |
| U3453 | 1.1 | 1.2 | 1.2 | 1.2 | 1.0 | 1.2 | 1.2 | 1.2 | 1.0 |
| U3457 | 2.2 | 2.3 | 2.4 | 2.2 | 1.9 | 2.1 | 2.3 | 2.2 | 1.8 |
| U3395 | 2.7 | 3.2 | 3.5 | 3.2 | 2.7 | 2.8 | 2.9 | 2.5 | 2.3 |
| U3398 | 1.5 | 1.7 | 1.8 | 1.8 | 1.5 | 1.6 | 1.6 | 1.6 | 1.5 |
| U3456 | 1.1 | 1.0 | 1.0 | 1.0 | 0.8 | 1.0 | 1.0 | 1.0 | 0.9 |
| U3459 | 2.9 | 2.6 | 2.8 | 2.6 | 2.2 | 2.4 | 2.5 | 2.5 | 2.0 |
| U0415 | 0.9 | 0.9 | 0.9 | 0.9 | 0.8 | 0.9 | 1.0 | 1.0 | 0.8 |
| U0432 | 1.8 | 1.5 | 1.5 | 1.5 | 1.3 | 1.4 | 1.4 | 1.4 | 1.3 |
| U0436 | 2.4 | 2.4 | 2.6 | 2.3 | 2.1 | 2.4 | 2.4 | 2.4 | 2.2 |
| U0438 | 3.9 | 3.7 | 4.2 | 3.7 | 3.2 | 3.8 | 4.2 | 3.9 | 3.6 |
| U0439 | 2.3 | 2.2 | 2.3 | 2.1 | 1.8 | 2.3 | 2.3 | 2.2 | 2.0 |
| U0440 | 5.8 | 4.8 | 5.4 | 5.2 | 4.4 | 4.6 | 4.8 | 4.3 | 5.2 |
| U0441 | 4.5 | 4.9 | 5.6 | 6.0 | 5.0 | 4.4 | 4.7 | 4.7 | 5.4 |
| U0442 | 1.8 | 1.7 | 1.8 | 1.7 | 1.5 | 1.8 | 1.8 | 1.8 | 1.6 |
| U3724 | 2.7 | 2.4 | 2.5 | 2.4 | 2.0 | 2.6 | 2.7 | 2.6 | 2.3 |
| U0849 | 2.4 | 2.3 | 2.4 | 2.1 | 1.8 | 2.3 | 2.4 | 2.2 | 2.0 |
| U0860 | 1.0 | 1.0 | 1.0 | 1.0 | 0.8 | 1.0 | 1.0 | 1.0 | 0.9 |
| U0861 | 2.8 | 2.9 | 3.0 | 2.8 | 2.6 | 3.0 | 3.0 | 2.9 | 3.0 |
| U0863 | 1.7 | 1.7 | 1.7 | 1.7 | 1.7 | 1.7 | 1.8 | 1.8 | 1.8 |
| U0875 | 2.2 | 2.0 | 2.2 | 2.1 | 1.6 | 2.0 | 2.0 | 2.0 | 1.7 |
| U0843 | 1.4 | 1.4 | 1.4 | 1.4 | 1.2 | 1.4 | 1.5 | 1.5 | 1.3 |
| U0848 | 1.3 | 1.4 | 1.4 | 1.4 | 1.2 | 1.3 | 1.4 | 1.4 | 1.2 |
| U0855 | 1.3 | 1.3 | 1.3 | 1.3 | 1.2 | 1.2 | 1.2 | 1.2 | 1.3 |
| U0867 | 3.2 | 2.9 | 3.2 | 2.8 | 2.5 | 3.0 | 3.1 | 3.0 | 2.9 |
| U1201 | 1.9 | 1.9 | 2.0 | 1.9 | 1.7 | 1.8 | 1.8 | 1.9 | 1.8 |
| U1202 | 1.3 | 1.3 | 1.3 | 1.3 | 1.2 | 1.4 | 1.4 | 1.4 | 1.2 |
| U1205 | 1.6 | 1.8 | 1.9 | 1.8 | 1.6 | 1.9 | 1.9 | 1.8 | 1.7 |
| U1207 | 1.9 | 1.9 | 2.0 | 1.8 | 1.5 | 1.9 | 1.9 | 1.7 | 1.7 |
| U1230 | 1.3 | 1.4 | 1.5 | 1.4 | 1.3 | 1.4 | 1.5 | 1.5 | 1.5 |
| U1198 | 2.2 | 2.1 | 2.2 | 2.1 | 1.9 | 2.0 | 2.0 | 2.0 | 2.3 |
| U1199 | 2.8 | 3.3 | 3.6 | 3.1 | 2.8 | 3.2 | 3.2 | 2.8 | 3.3 |
| U1218 | 3.0 | 2.6 | 2.9 | 2.9 | 2.7 | 2.8 | 3.1 | 3.1 | 3.2 |
| U1225 | 2.2 | 2.3 | 2.3 | 2.1 | 1.9 | 2.6 | 2.4 | 2.2 | 2.2 |
| U1575 | 1.4 | 1.3 | 1.3 | 1.3 | 1.4 | 1.4 | 1.4 | 1.4 | 1.4 |
| U1579 | 1.5 | 1.7 | 1.7 | 1.7 | 1.5 | 1.8 | 1.8 | 1.7 | 1.5 |
| U1649 | 0.9 | 0.8 | 0.8 | 0.8 | 0.8 | 0.9 | 0.9 | 0.9 | 0.8 |
| U1576 | 2.2 | 2.1 | 2.1 | 2.1 | 2.1 | 2.3 | 2.3 | 2.3 | 2.2 |
| U1581 | 1.7 | 1.7 | 1.7 | 1.8 | 1.9 | 1.7 | 1.8 | 1.8 | 1.7 |
| U1599 | 2.0 | 1.7 | 1.8 | 1.8 | 2.0 | 2.0 | 2.1 | 2.1 | 2.0 |
| U1600 | 3.5 | 3.2 | 3.4 | 3.4 | 3.7 | 3.9 | 4.2 | 3.5 | 3.7 |
| U4471 | 1.5 | 1.6 | 1.7 | 1.6 | 1.5 | 1.7 | 1.7 | 1.7 | 1.7 |
| U4757 | 2.0 | 2.1 | 2.1 | 2.0 | 1.8 | 2.0 | 2.0 | 2.1 | 2.0 |
| U4767 | 2.6 | 2.4 | 2.5 | 2.6 | 2.0 | 2.6 | 2.6 | 2.5 | 2.3 |
| U4772 | 2.5 | 2.7 | 2.8 | 2.5 | 2.6 | 2.8 | 2.8 | 2.9 | 2.5 |
| U4801 | 1.3 | 1.4 | 1.4 | 1.4 | 1.2 | 1.5 | 1.5 | 1.4 | 1.2 |
| U4737 | 2.9 | 2.6 | 2.8 | 2.7 | 2.3 | 2.7 | 2.9 | 2.8 | 2.5 |
| U4752 | 1.4 | 1.5 | 1.6 | 1.5 | 1.3 | 1.5 | 1.5 | 1.5 | 1.4 |
| U5133 | 0.9 | 0.9 | 0.9 | 0.9 | 0.7 | 1.0 | 1.0 | 1.0 | 0.8 |
| U5173 | 1.1 | 1.2 | 1.2 | 1.2 | 1.1 | 1.2 | 1.2 | 1.2 | 1.0 |
| U5175 | 1.7 | 1.8 | 1.9 | 1.8 | 1.7 | 1.9 | 1.9 | 1.9 | 1.7 |
| U5178 | 2.3 | 2.2 | 2.3 | 2.1 | 1.9 | 2.6 | 2.9 | 2.8 | 2.0 |
| U5183 | 2.9 | 2.6 | 2.8 | 2.6 | 2.3 | 3.6 | 3.9 | 3.7 | 3.0 |
| U5158 | 5.5 | 5.1 | 5.9 | 5.7 | 5.8 | 6.0 | 6.6 | 7.1 | 7.0 |
| U5169 | 2.6 | 2.9 | 3.2 | 3.2 | 3.2 | 3.2 | 3.4 | 3.6 | 3.7 |
| U5190 | 2.8 | 2.7 | 2.8 | 2.9 | 2.8 | 3.2 | 3.4 | 3.5 | 3.2 |
| U5193 | 3.1 | 3.0 | 3.1 | 3.0 | 2.9 | 3.6 | 3.7 | 3.7 | 3.4 |
| U5589 | 1.6 | 1.8 | 1.9 | 1.8 | 1.6 | 1.9 | 2.0 | 1.8 | 1.5 |
| U5592 | 1.1 | 1.2 | 1.2 | 1.2 | 1.1 | 1.4 | 1.3 | 1.3 | 1.4 |
| U5593 | 1.7 | 1.8 | 1.9 | 1.8 | 1.6 | 1.8 | 1.9 | 1.8 | 1.7 |
| U5565 | 2.7 | 3.2 | 3.3 | 3.3 | 3.1 | 3.5 | 3.5 | 3.6 | 3.5 |
| U5591 | 2.0 | 2.2 | 2.3 | 2.3 | 2.1 | 2.3 | 2.3 | 2.1 | 2.3 |
| U5594 | 2.3 | 2.6 | 2.8 | 2.8 | 2.8 | 2.8 | 2.8 | 3.0 | 3.0 |
| U5597 | 3.3 | 3.3 | 3.6 | 3.6 | 3.1 | 4.1 | 4.0 | 4.3 | 4.0 |
| U5993 | 1.0 | 0.9 | 0.9 | 0.9 | 0.8 | 1.0 | 1.0 | 1.0 | 0.8 |
| U6017 | 1.0 | 0.9 | 1.0 | 1.0 | 0.8 | 0.9 | 0.9 | 0.9 | 0.8 |
| U6056 | 1.0 | 1.0 | 1.0 | 1.0 | 0.9 | 1.0 | 1.0 | 1.0 | 0.9 |
| U5992 | 1.4 | 1.4 | 1.4 | 1.4 | 1.3 | 1.3 | 1.4 | 1.4 | 1.3 |
| U6047 | 2.3 | 2.3 | 2.4 | 2.3 | 2.0 | 2.2 | 2.3 | 2.3 | 2.2 |
| U6060 | 1.9 | 2.1 | 2.2 | 2.2 | 2.0 | 2.3 | 2.0 | 2.0 | 2.1 |
| U6065 | 3.1 | 2.8 | 2.9 | 2.8 | 2.7 | 3.0 | 3.1 | 2.9 | 2.8 |
| U6928 | 1.2 | 1.2 | 1.2 | 1.2 | 1.2 | 1.2 | 1.2 | 1.2 | 1.1 |
| U6929 | 1.2 | 1.2 | 1.2 | 1.2 | 1.1 | 1.2 | 1.2 | 1.2 | 1.0 |
| U6951 | 1.5 | 1.5 | 1.5 | 1.5 | 1.5 | 1.6 | 1.7 | 1.6 | 1.4 |
| U6977 | 1.3 | 1.3 | 1.3 | 1.3 | 1.2 | 1.3 | 1.4 | 1.4 | 1.1 |
| U6936 | 2.4 | 2.5 | 2.4 | 2.6 | 3.2 | 2.6 | 2.6 | 2.7 | 2.6 |
| U6938 | 2.1 | 2.1 | 2.1 | 2.2 | 2.3 | 2.3 | 2.3 | 2.3 | 2.3 |
| U6972 | 2.4 | 2.4 | 2.5 | 2.4 | 2.5 | 2.8 | 2.8 | 2.8 | 2.5 |
| U6987 | 5.1 | 4.5 | 4.4 | 5.0 | 5.5 | 5.7 | 5.4 | 5.7 | 7.0 |
| U7316 | 1.2 | 1.1 | 1.1 | 1.1 | 1.1 | 1.3 | 1.3 | 1.3 | 1.1 |
| U7321 | 1.5 | 1.4 | 1.4 | 1.4 | 1.5 | 1.6 | 1.6 | 1.6 | 1.5 |
| U7324 | 1.3 | 1.2 | 1.3 | 1.2 | 1.2 | 1.4 | 1.4 | 1.4 | 1.2 |
| U7317 | 2.0 | 1.6 | 1.7 | 1.7 | 1.6 | 1.9 | 1.9 | 1.8 | 1.6 |
| U7318 | 2.8 | 2.7 | 2.9 | 2.9 | 2.6 | 3.3 | 3.4 | 3.3 | 2.7 |
| U7320 | 2.0 | 1.9 | 1.9 | 1.9 | 2.2 | 2.0 | 2.1 | 2.1 | 2.2 |
| U7322 | 1.8 | 1.7 | 1.7 | 1.7 | 1.5 | 1.7 | 1.8 | 1.7 | 1.4 |
| U7708 | 1.6 | 1.6 | 1.6 | 1.6 | 1.6 | 1.7 | 1.7 | 1.7 | 1.7 |
| U7713 | 1.4 | 1.6 | 1.6 | 1.6 | 1.5 | 1.6 | 1.6 | 1.6 | 1.5 |
| U7727 | 1.7 | 1.7 | 1.7 | 1.8 | 1.7 | 1.9 | 1.9 | 1.9 | 1.9 |
| U7794 | 1.9 | 1.8 | 1.9 | 1.8 | 1.6 | 1.7 | 1.8 | 1.7 | 1.6 |
| U7707 | 2.2 | 2.2 | 2.3 | 2.3 | 2.3 | 2.3 | 2.3 | 2.3 | 2.2 |
| U7710 | 2.3 | 2.5 | 2.6 | 2.7 | 2.8 | 2.7 | 2.9 | 3.0 | 3.0 |
| U7724 | 2.4 | 2.4 | 2.5 | 2.6 | 2.7 | 2.7 | 2.7 | 2.8 | 2.9 |
| U7738 | 2.4 | 2.3 | 2.4 | 2.5 | 2.2 | 2.4 | 2.5 | 2.6 | 2.3 |
| U8559 | 1.6 | 1.4 | 1.4 | 1.4 | 1.3 | 1.6 | 1.7 | 1.6 | 1.3 |
| U8570 | 1.2 | 1.2 | 1.2 | 1.2 | 1.3 | 1.2 | 1.2 | 1.2 | 1.3 |
| U8575 | 0.9 | 0.8 | 0.8 | 0.8 | 0.8 | 0.9 | 0.9 | 0.9 | 0.8 |
| U8555 | 2.6 | 2.4 | 2.5 | 2.6 | 2.6 | 2.9 | 3.1 | 3.0 | 2.6 |
| U8558 | 2.3 | 2.2 | 2.3 | 2.3 | 2.2 | 2.3 | 2.3 | 2.4 | 2.4 |
| U8563 | 2.2 | 2.3 | 2.3 | 2.4 | 2.3 | 2.4 | 2.4 | 2.5 | 2.5 |
| U9031 | 2.1 | 2.4 | 2.3 | 2.3 | 2.5 | 2.6 | 2.4 | 2.3 | 2.4 |
| U9032 | 1.7 | 1.7 | 1.7 | 1.7 | 1.6 | 1.9 | 1.9 | 1.7 | 1.5 |
| U9040 | 1.4 | 1.4 | 1.4 | 1.4 | 1.2 | 1.4 | 1.4 | 1.3 | 1.1 |
| U9034 | 3.0 | 2.9 | 2.8 | 3.0 | 4.0 | 3.4 | 3.4 | 3.5 | 3.8 |
| U9039 | 2.7 | 3.0 | 3.2 | 3.1 | 3.1 | 3.2 | 3.2 | 3.2 | 3.3 |
| U9049 | 3.5 | 3.3 | 3.5 | 3.5 | 3.5 | 3.6 | 3.8 | 3.6 | 3.7 |
| U9055 | 2.4 | 2.1 | 2.1 | 2.2 | 2.1 | 2.4 | 2.4 | 2.4 | 2.1 |
| U0048 | 1.8 | 1.8 | 1.8 | 1.8 | 1.7 | 1.9 | 2.0 | 2.0 | 1.8 |
| U0050 | 1.8 | 1.7 | 1.8 | 1.8 | 1.7 | 1.9 | 2.0 | 2.0 | 1.7 |
| U0056 | 1.6 | 1.5 | 1.5 | 1.5 | 1.4 | 1.8 | 1.8 | 1.7 | 1.5 |
| U0047 | 2.1 | 1.7 | 1.8 | 1.8 | 1.6 | 2.0 | 2.1 | 2.0 | 1.7 |
| U0058 | 3.2 | 2.8 | 2.9 | 3.0 | 3.0 | 3.3 | 3.4 | 3.2 | 3.3 |
| U0060 | 2.2 | 2.1 | 2.1 | 2.2 | 2.1 | 2.2 | 2.2 | 2.2 | 2.3 |
| U0062 | 2.8 | 2.6 | 2.7 | 2.8 | 2.7 | 3.0 | 3.2 | 3.2 | 2.9 |

TABLE 9
COMPARATIVE RESULTS

| Comparison | | | | | | | |
|---|---|---|---|---|---|---|---|
| on | n | r | m | b | Std. Error | Ng | Lassen |
| GInr vs | 129 | 0.997 | 0.879 | 0.163 | 0.079 | 7/129 = | Delta <= 0.4\|5 @ 96.1% |
| GATFa | | | | | | 5.4% | Delta <= 0.7\|2 @ 98.4% |
| GInr vs | 129 | 0.986 | 0.948 | 0.078 | 0.162 | 3/129 = | Delta <= 0.4\|4 @ 96.9% |
| GATFz | | | | | | 2.3% | Delta <= 0.7\|2 @ 98.4% |
| GInr vs | 129 | 0.974 | 0.935 | 0.413 | 0.221 | 20/129 = | Delta <= 0.4\|16 @ 87.6% |
| GATFt2 | | | | | | 15.5% | Delta <= 0.7\|4 @ 96.9% |
| MInr vs | 129 | 0.996 | 0.921 | 0.122 | 0.092 | 9/129 = | Delta <= 0.4\|2 @ 98.4% |
| MATFa | | | | | | 7.0% | Delta <= 0.7\|0 @ 100.0% |
| MInr vs | 129 | 0.989 | 0.908 | 0.190 | 0.155 | 7/129 = | Delta <= 0.4\|4 @ 96.9% |
| MATFz | | | | | | 5.4% | Delta <= 0.7\|2 @ 98.4% |
| MInr vs | 129 | 0.983 | 0.893 | 0.491 | 0.193 | 8/129 = | Delta <= 0.4\|13 @ 89.9% |
| MATFt2 | | | | | | 6.2% | Delta <= 0.7\|4 @ 96.9% |

Table 9 provides comparative data for the ATFa, ATFz and ATFt2 and INR values calculated by the WHO method for each respective location, with GInr representing one location for these traditionally WHO determined values, and MInr representing values based on data obtained at the other location. The values identified as ATFz and ATFt2, such as, GATFt2 and MATFt2, and GATFz and MATFz, represent anticoagulant therapy factors derived from the expressions (1) through (9) above, inclusive of expressions (5.1) and (8.1).

Further comparative results are provided in Table 10 to illustrate the effect of prothrombin time (PT) on INR values. Table 10 provides a comparison based on data from Table 3, and provides INR values for PT's of PT=PT (under the heading “INR”), PT=PT+0.5 (under the heading “+0.5”), PT=PT+1.0 (under the heading “+1.0”), PT=PT+1.5 (under the heading “+1.5”), and PT=+2.0 (under the heading “+2.0”). The new anticoagulation therapy factor (ATFt2) was compared with the WHO method for determining ATF. The WHO method utilizes the mean prothrombin time of 20 presumed normal patients. The thromboplastin reagents list MNPT “expected ranges” listed in the accompanying thromboplastin-reagent (Tp) brochures. These brochures acknowledge that MNPT differences are inevitable because of variations in the 20 “normal donor” populations. Geometric, rather than arithmetic mean calculation limits MNPT variation somewhat, but simulated 0.5 second incremented increases over a total 2.5 second range, show ever-increasing INR differences notably at higher INR levels. To exemplify this, Table 10 shows these changes with Thromboplastin C Plus (which has a manufacturer's reported ISI=1.74 and MNPT=9.89 seconds) in POTENS+.

TABLE 10

| ID | PT | INR | +0.5 | +1.0 | +1.5 | +2.0 |
|---|---|---|---|---|---|---|
| WEC | 9.8 | 1.0 | 0.9 | 0.8 | 0.8 | 0.7 |
| A095 | 12.5 | 1.5 | 1.4 | 1.3 | 1.2 | 1.1 |
| A191 | 14.8 | 2.0 | 1.9 | 1.7 | 1.6 | 1.5 |
| A112 | 16.9 | 2.5 | 2.3 | 2.2 | 2.0 | 1.8 |
| A208 | 18.6 | 3.0 | 2.8 | 2.5 | 2.3 | 2.2 |
| A020 | 20.3 | 3.5 | 3.2 | 3.0 | 2.7 | 2.5 |
| A164 | 21.9 | 4.0 | 3.7 | 3.4 | 3.1 | 2.9 |
| A093 | 24.5 | 4.9 | 4.5 | 4.1 | 3.8 | 3.5 |
| A055 | 26.5 | 5.6 | 5.1 | 4.7 | 4.4 | 4.0 |
| A090 | 28.5 | 6.3 | 5.8 | 5.3 | 4.9 | 4.6 |
| R091 | 32.2 | 7.8 | 7.2 | 6.6 | 6.1 | 5.7 |
| A058 | 33.8 | 8.5 | 7.8 | 7.2 | 6.6 | 6.2 |

Since the in-house determined MNPT would continue with that Tp lot, intralaboratory results would be relatively unaffected. However, between laboratory INR agreements, or interlab results, are compromised. As a denominator, considering the expression used to derive the MNPT, such as expression (B), above, MNPT is, of course, less problematic for INRs than the exponent, ISI. Comparative results, showing interlab results, are provided in Table 11. ATFt is seen to be numerically equal to WHO/INRs determined in both analytical instruments, namely, the MDA-Electra 9000C and the POTENS+. Identical computer bits derived in POTENS+from the absorbances creating the thrombin-fibrinogen-fibrin clotting curve are used for the POTENS+WHO/INR and ATFt (NO ISI, NO MNPT) determinations. MNPT is, of course, still necessary for the WHO method. For ATFt, Zero Order Kinetics Line's slope is extended in both directions to intersect with the Tp-plasma baseline and the absorbance at total fibrin formation. The sum of this interval and the time from the Tp injection to the beginning of Zero Order Kinetics (T₂S) is Value 1. Value 2 is T₂S/100e. “e” is the Natural Logarithm, base 2.71828. ATFt=(Value 1)*(Value 2), in accordance with expression (4) herein (and the expression (8.1) for ATFt2).

Table 11 provides statistical comparisons for results obtained using two POTENS+coagulometers (one designated as GINR and another designated as MINR), and using a Bio Merieux MDA-180 coagulometer (designated as AINR). The POTENS+, WHO/INRs, INRzs, and ATFts and the MDA-180 (AINR) WHO/INRs are compared. Statistical data and Bland-Altman plot data demonstrate that the new anticoagulant therapy factor ATFt may replace WHO/INR and provide results which are within the parameters of traditional therapeutic or reference ranges.

TABLE 11

| | | “r” | “m” | “b” | StdErr | StdDev | mY | mX | My/mX |
|---|---|---|---|---|---|---|---|---|---|
| AINR | | | | | | | | | |
| vs | GINR | 0.937 | 0.872 | 0.290 | 0.388 | 1.148 | 2.169 | 2.155 | 1.007 |
| | GATFz | 0.941 | 1.119 | −0.208 | 0.378 | 1.022 | 2.169 | 2.124 | 1.021 |
| | GATFt2 | 0.951 | 1.003 | 0.146 | 0.343 | 1.081 | 2.169 | 2.016 | 1.076 |
| | MINR | 0.950 | 1.018 | −0.126 | 0.349 | 1.070 | 2.169 | 2.253 | 0.963 |
| | MATFz | 0.943 | 1.020 | −0.040 | 0.371 | 1.065 | 2.169 | 2.167 | 1.001 |
| | MATFt2 | 0.937 | 0.872 | 0.290 | 0.388 | 1.148 | 2.169 | 2.155 | 1.007 |
| MINR | | | | | | | | | |
| vs | GINR | 0.971 | 1.036 | 0.039 | 0.247 | 1.001 | 2.253 | 2.136 | 1.055 |
| MINRz | | | | | | | | | |
| vs | GINRz | 0.984 | 1.082 | −0.132 | 0.186 | 0.978 | 2.167 | 2.124 | 1.020 |
| MINRt2 | | | | | | | | | |
| vs | GINRt2 | 0.979 | 1.110 | −0.083 | 0.242 | 1.123 | 2.155 | 2.016 | 1.069 |

The linear regression analysis expression y=mx+b, when solved for the slope, m, is expressed as (y−b)/x. This is biased, so the expression is y/x is when b is equal to zero. The comparison in Table 11, above, provides comparative data for mean y (mY) and mean x (mX) values, including the slope mY/mX. The use of mY/mX is used to provide comparative results.

In another embodiment, an article may be provided to derive an anticoagulant therapy factor (ATF). The article may comprise stored instructions on a storage media which can be read and processed with a processor. For example, the computer may be provided with a stored set of instructions, or chip, which is programmed to determine a new ATF for the spectral data obtained from the coagulation activity of a sample. For example, the computer chip may be preprogrammed with a set of instructions for cooperating with the output of a photodetection device, such as, the device shown and described in FIG. 1, which provides electrical data to said computer processor and/or storage device as a function of the optical density for a sample being analyzed. The chip may be employed in, or used with, an apparatus having input means and storage means for storing data. The set of instructions on the chip includes instructions for carrying out the steps of determining one or more anticoagulant therapy factors based on the expressions (1) through (9), inclusive of expressions (5.1) and (8.1).

According to alternate embodiments of the invention, the method and apparatus may involve the detection of additional prothrombotic abnormalities (or disorders) in a patient. According to preferred embodiments of the invention, methods and apparatus for conducting a determination relating to a hypercoagulation condition are provided. According to preferred embodiments of the invention, the prothrombotic abnormality evaluated is hypercoagulability. In other words, the hypercoagulable condition in an individual may be evaluated in a relatively short duration of time. The method and apparatus include analyzing a blood sample as a clotting activator, such as thromboplastin, is added, to track the change in the optical density corresponding to a blood component, such as fibrin formation based on the fibrinogen content of the blood sample. The absorbance curve develops when optically-clear fibrinogen is converted into turbid fibrin after the clotting agent. Preferred embodiments of the method and apparatus evaluate fibrinogen conversion activity.

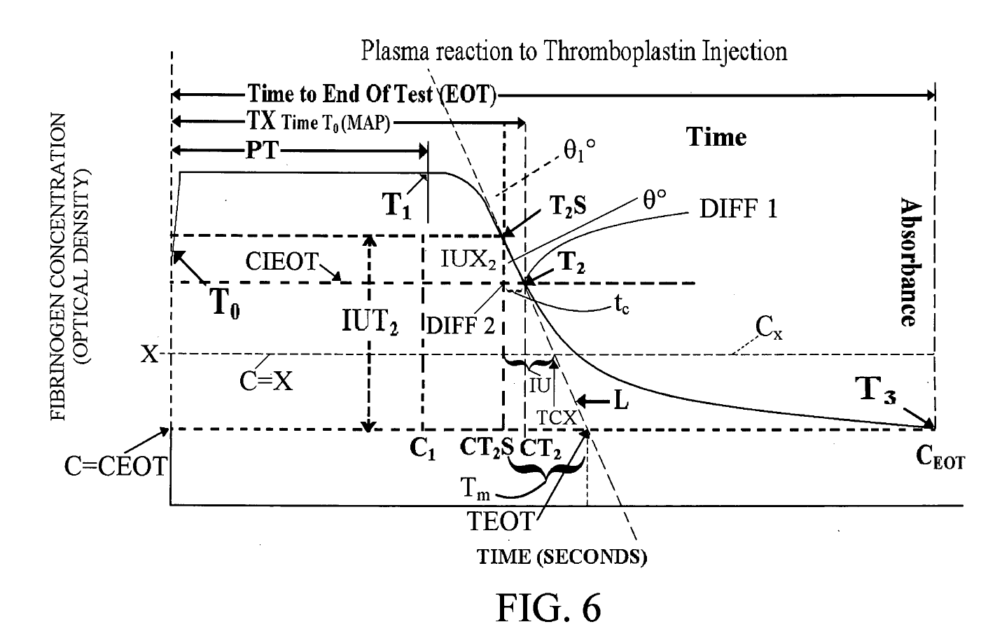

The method involves taking readings and using the readings, such as, for example, by recording the readings over a particular time interval or duration. The absorbance value (which may be measured in instrument units) is plotted against time (which may be measured in seconds or fractions of seconds). The method involves the collection and processing of the data. The data may be represented in a clotting curve plot. A plot is formed which has a slope where the maximum acceleration of the rate of conversion of fibrinogen to fibrin occurs. According to preferred embodiments, the method and apparatus utilize the tangent of the slope to derive a zero order kinetic. According to preferred embodiments, a portion of the zero order slope line (such as the zero order line L shown in FIG. 3 and also in FIGS. 6-8) may be utilized to derive an indicator corresponding with a coagulation state, such as, a hypercoagulable condition. Through the utilization of the slope, correspondence with a hypercoagulable condition of an individual may be evaluated. The slope may be used in conjunction with values relating to trigonometric functions, such as a tangent of an angle, to provide an indicator of a hypercoagulable condition.

Referring to FIG. 6, the zero order line L is illustrated passing through the horizontal line c=cEOT representing the instrument value (e.g., a value relating to the fibrinogen to fibrin conversion) at the end of the test. The point of intersection of the horizontal line c=cEOT with the line L provides a corresponding time of TEOT, or theoretical end of test. The difference between that time value TEOT and the time value T2S may be assigned to represent a first trigonometric function value Tm. A second trigonometric function value may be represented by the difference (e.g., in instrument units) between the unit value (cT2S) corresponding with the time to start T2S and the unit value (cEOT) corresponding with the end of the test (T3), or, in other words, expressed as the differential IUT, as shown in FIG. 6.

The derivation of a hypercoagulability indicator may be carried out by evaluating trigonometric values associated with the slope. According to a preferred embodiment of the method, an angle may be derived as an indicator of potential prothrombotic abnormality. FIG. 6 illustrates the method where the prothrombotic condition to be indicated is a hypercoagulability condition. The angle θ° may be derived using a tangent function. For example, a tangent function may utilize values derived in conjunction with the clotting curve slope, such as the line L. According to preferred embodiments, the first trigonometric function value Tm and second trigonometric function value IUT may be used to determine a tangent corresponding to an angle θ°. FIG. 6 illustrates an example of a clot slope curve where a tangent is determined for the angle θ° based on the expression:

tangent θ°=Tm/IUT (10)

where Tm=TEOT−T2S, and wherein IUT=cT2S−cEOT.

Though the actual absorbance values themselves would not be less than zero, it is conceivable that the line representing t=T2S may be extended below the abscissa (the line t=0), and the additive absolute value of c determined (e.g., where c=a negative ordinate) adding the absolute value of the ordinate C₁=|−X| with the value C_{T2S}. The value of the sum of C₁ and C_{T2S} may be determined to yield a trigonometric function value, an adjacent leg (AL). The opposite leg (OL) may be determined by T_{(CI/L)}−T2S, where T_{(CI/L)} is the time where L crosses (i.e., intersects with) the line c=C₁. According to preferred embodiments, the determination of a hypercoagulable condition may be made using the slope of the clotting curve and a reference, namely T=T2S. In other words determining absorbance values for a sample during a coagulation reaction at time T2S and T2 may be utilized by the present method and apparatus to determine the presence of a hypercoagulable condition.

The method involves reacting a sample of a patient's blood or blood components with a clotting agent, such as thrombin. The clotting curve in FIG. 6 illustrates the introduction of the clotting agent, which, for example, corresponds with time T₀. A zero order kinetic line having a slope based on absorbance and time values for the clotting activity of the sample, is determined. The zero order line may be determined as discussed herein, including as shown in conjunction with FIGS. 3, 4 and 5. The slope of the zero order kinetic line may be different from sample to sample, though some samples may exhibit identical or similar slopes. The slope is evaluated for a corresponding patient sample whose clotting analysis data forms the slope. Information obtained from the clotting curve data, including data forming the slope, is compared with ranges or values indicative of a condition of clinically unremarkable (or normal) coagulability, or ranges or values considered to correspond to a prothrombotic abnormality, such as the condition of hypercoagulability. The determination may be carried out and results obtained within a matter of seconds. The hypercoagulability determination may be obtained within a minute, and more preferably may be determined within seconds. For example, hypercoagulability determinations according to the method and apparatus described herein have been accomplished within as little as thirty seconds.

The invention also includes an apparatus useful for carrying out analyses on a sample of a patient's blood or blood components. According to a preferred embodiment, the apparatus may include a light source and a photocell for detecting the light emitted from the light source. The light source may be provided to generate energy of a particular wavelength, such as, for example, 660 nm, or other useful wavelength or spectral range, which is capable of detecting the formation of fibrin. The sample may be placed between the path of the light source and a photocell detector. According to preferred embodiments, the wavelength selected is capable of distinguishing the fibrin formation. The apparatus also may include or be linked with a processor, such as a computer, which may record absorbance values over a time interval, where the absorbance values represent the fibrin level in the sample at the corresponding given time values. According to the examples shown in FIGS. 2-8, the recorded absorbance values are collected and displayed to form the clotting curves. The clotting curves are representative of the absorbance and time data for the fibrin conversion which is commenced by the addition of an agent to a sample containing fibrinogen. The apparatus, according to other embodiments, may include means which may be used to collect and process the data ascertained with a spectrophotometer. One embodiment includes an article for determining the presence of a hypercoagulable condition. The article preferably includes storage media with stored instructions which may be read and processed with a processor to determine whether a slope value obtained for clotting curve data corresponds to to a value indicative of a hypercoagulable condition. Another embodiment includes an apparatus having a processor and a computer chip (or other readable media) preprogrammed with a set of instructions for cooperating with the output of a photodetection device which provides electrical data to the processor as a function of the optical density for a sample being analyzed. The apparatus may include input means for inputting information or making selections and storage means for storing data. The set of instructions includes instructions for determining a prothrombotic abnormality or disorder, such as, for example a hypercoagulable condition. The instructions may be programmed to determine an angle and undertake an angle analysis in accordance with the steps described herein:

It is noted that the prothrombin times (PT's) for a blood sample of a patient with a hypercoagulable condition may be in the shorter range, as compared with the mean or average PT, (that is a PT that is presumed to correspond with that of a patient considered to have normal coagulation). The shorter PT may be an indicator of the possibility of a hypercoagulable condition, but the PT being short does not necessarily mean that the patient has a hypercoagulability state.

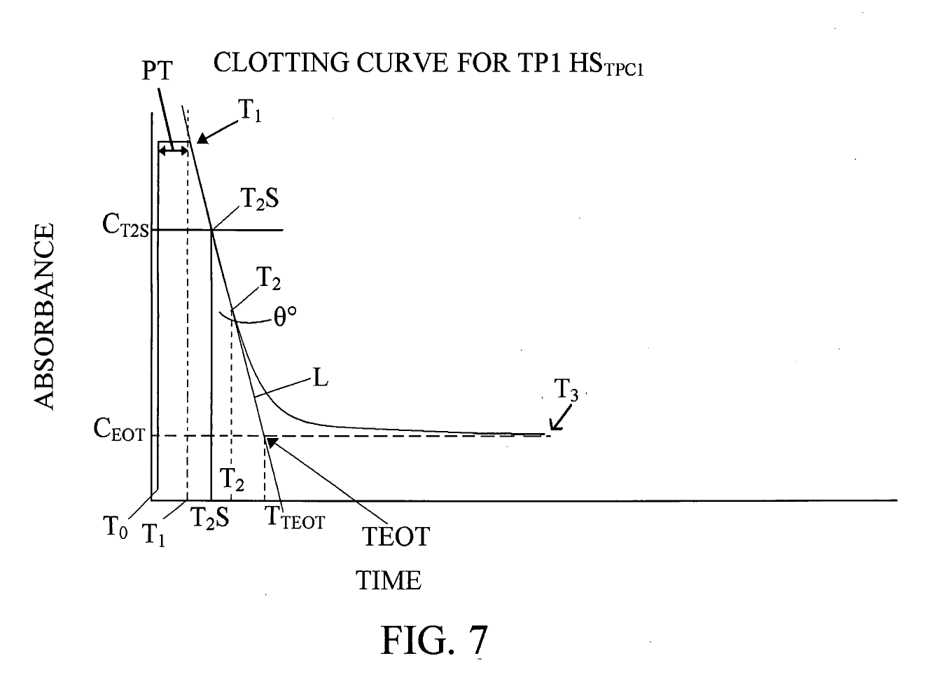

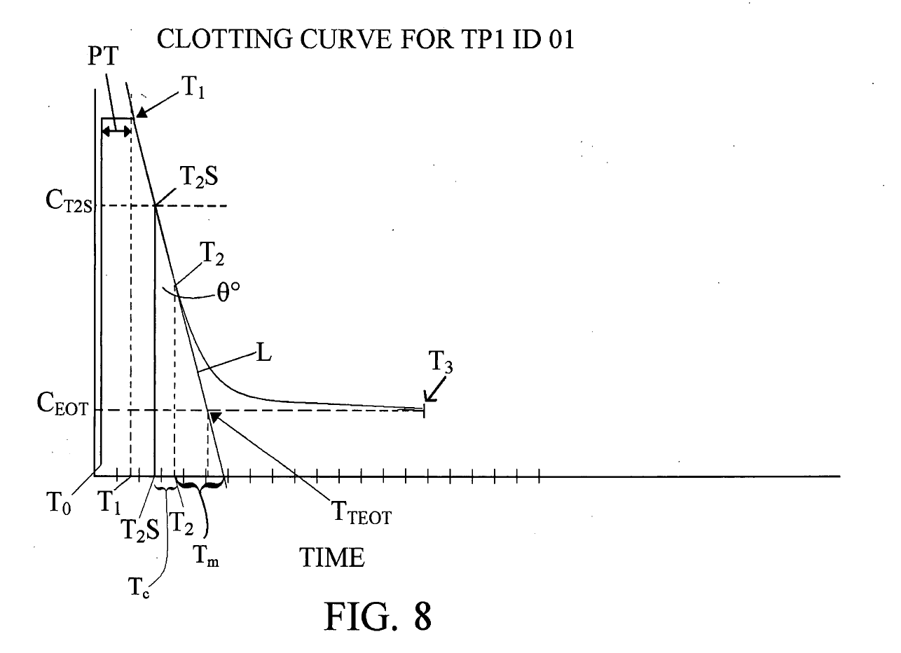

The hypercoagulability condition is illustrated in relation to clotting curves showing both the presumed normal coagulation patients and those exhibiting the characteristics of a hypercoagulable condition. As discussed herein, from the results of the absorbance values, expressed in units, and the corresponding time values of the clotting reaction, a zero order kinetic slope is obtained. The indicator value is derived from the zero order line. Examples of the slopes obtained are illustrated in the clotting plots of FIGS. 2-8. According to embodiments of the invention, the method for determining hypercoagulability may be illustrated with reference to the plots of FIGS. 6-8. FIG. 7 illustrates correspondence of a hypercoagulable condition, while FIG. 8 illustrates correspondence with a presumed normal coagulation state (that is, a state which is not hypercoagulable).

As illustrated in FIGS. 6-8, the angle θ° is completed by XT (or T2) on the graph. XT (or T2) represents a maximum acceleration point of the rate of conversion of fibrinogen to fibrin. The line L may be completed upon the determination of XT (or T2), as the line L is formed based on the times T2S and T2. Upon completion of the line L, the angle that the line T2S makes with L is formed. The end of the test time T3 may be used to provide a horizontal line c=cEOT, and thereby derive a theoretical end of test TEOT which is considered to be the time corresponding to the intersection of L and c=cEOT.

According to an alternate embodiment of the invention, in order to provide even more rapid results, the method, upon ascertaining the clotting curve data to form the line L, may utilize an implied end of test line, IEOT, which corresponds with c=cIEOT. The implied end of test line IEOT may be selected from the values cIEOT=x, where x is: 0<x<c_{T2S}. The time differential between a first differential reference (DIFF1) tIEOT/L (which is at the intersection of cIEOT and L) and a second differential reference (DIFF2) (which is at the intersection of cIEOT and t=T2S) may be used to determine an opposite leg (OL) of the tangent of the angle θ° to be determined.

Opposite leg (OL)=DIFF1−DIFF2

An adjacent leg (AL) is also determined. A unit differential (expressed in instrument units) forms an adjacent leg for determining the tangent of the angle θ°. The unit differential for the adjacent leg (AL) may be determined as the vertical distance (in instrument units) between c=x and cT2S, that is cT2S less x (where c=x).

Adjacent leg (AL)=CT2S−x (12)

Accordingly, tangent θ°=(DIFF1−DIFF2)/(CT2Sx) (13)

FIG. 6 illustrates an example of the application of expression (13) where x is represented as x=T2, where DIFF1−DIFF2 is represented as OL (indicated as tm on FIG. 6), and AL=cT2S−Cx, which in this example is IUX (that is, cT2S−cT2). The example therefore illustrates an arrangement where Cx is the absorbance value (in instrument units) at time T2. The tangent of the angle θ° may be determined by taking T2S-T2/IUX.

Alternately, the method may derive an angle θ° using the opposite and adjacent legs formed from a horizontal line segment c=X, where c lies between cT2S and zero, and c=X is The expression may be represented as:

tangent θ°=Tval/IU (11)

where Tval is a time differential between time T2S and a point TcX, where TcX is the time corresponding with the intersection of c=X and L. IU represents the instrument units between cT2S (which is an absorbance value at time T2S) and c=x (which is an absorbance value at time TcX).

Conversely, the line L may be extended above the clotting curve to intersect with the vertical line t=T1 forming the vertex of an angle, with the vertical line t=T2S forming an adjacent leg for determining the tangent of the angle θ₁° (see FIG. 6).

The slope of the clotting curve may be derived by equations described herein and represents the increase in the rate of fibrinogen conversion. The tangent line L also may be determined using the methods and apparatus described herein. Through the use of the clotting curve data, the indicator values indicative of and/or corresponding with a hypercoagulable condition in an individual may be determined.

One or more reference parameters may be established to serve as a measure against which patient samples may be compared. A patient's blood sample is used to react with a clotting activator, and clotting curve data is collected. The methods described herein for reacting the blood sample with a clotting agent that activates the fibrinogen conversion, such as, for example, thromboplastin C Plus, BTP or Innovin may be employed in conjunction with the hypercoagulability determination. The methods and apparatus used to obtain a clotting curve for a sample may be applied in order to facilitate the hypercoagulable determination. The hypercoagulability condition is assigned by the evaluation of an indicative expression ascertained through the sample data.

Referring to FIGS. 7 and 8, the angle θ° may be used to provide an indicator of a prothrombotic condition. According to the preferred embodiments, the prothrombotic condition indicated is a hypercoagulable condition. A indication that is considered positive for a hypercoagulable condition is a reported angle deviation. The angle deviation may be defined as a deviation from the angle ascertained for blood samples of persons having presumed normal coagulation conditions (such as a mean normal angle derived from a group of people with presumed normal coagulation). The test according to the present method and apparatus may be used to determine whether a hypercoagulable condition is present in the person whose blood sample produces'the corresponding angle deviation. Conversely, the results of the angle deviation test may be applied to exclude persons who are indicated not to have a hypercoagulable condition (where testing fails to show a sufficient angle deviation). For example, the exclusion of persons from a class or category of potential or known hypercoagulable condition candidates may be used to determine a course of treatment or testing of that person, including the exclusion from further testing or treatment that is not deemed to be necessary. The method facilitates administering treatment or further testing, or potentially both, to a more particularized group, rather than the entire patient class. The test according to the present methods and apparatus may be carried out to provide results of whether hypercoagulable condition is indicated, preferably prior to the time frame where further testing (or even treatment) is required to be given to a patient who, prior to the present hypercoagulability condition test, was suspected of one or more prothrombotic conditions. The test results achieved with the present methods and apparatus may be realized within a matter of seconds.

According to embodiments of the method and apparatus, the angle deviation in its simplest expression may be considered to be a statistically significant deviation from the angle determined for the clotting data: of blood samples of persons presumed to have a normal coagulation condition. These presumed normal coagulation condition patients may be patients who do not exhibit hypercoagulable or hypocoagulable conditions, as may have been previously determined through more expensive and time consuming tests, or by presenting a symptom confirming the condition. The angle deviation may be circumscribed to be a percent deviation from a mean normal angle value (e.g., that obtained for samples from presumed normal coagulation condition persons). The angle deviation, according to some embodiments, may be a measure of standard deviations from the mean normal angle value. If one standard deviation is within the sampling or instrumentation error or range, then a different value may be selected. For example, two standard deviations may be used to categorize test results for angles which are considered to be discrepant or considered an angle deviation (indicative of a hypercoagulable condition for that sample). According to alternate embodiments, angles equal to or greater than three standard deviations may be used to categorize test results that correspond with a condition considered to be a hypercoagulable condition.

According to preferred embodiments, a standard may be established. The blood or blood component samples from presumed normal persons may be run through the test in order to derive a standard for the angle. The standardization angle data (SAD) may be stored. A computer with, or operating linked to, suitable storage means, such as a hard drive, or other suitable apparatus, including those described herein, may store the standardization angle data (SAD). In addition, a standardized sample solution, which may be a blood sample, or other sample containing fibrinogen, may be provided as an instrument calibration standard. The standard may be used to provide a standard angle for a particular instrument. The standard angle may be used as a reference measurement against which angle deviations of patient samples undergoing the hypercoagulability test may be determined. Alternately, or in conjunction with the calibration standard samples, the standard data may be stored on a device, such as a computer memory element. The data may be stored in electronic or other digital form. Though the method may be carried out through a physical or manual manipulation and comparison of the clot slope data, including the expression of that data in the form of a clotting curve. A module may be provided that contains data and may include software with instructions for instructing a processor to record and compare data from a sample analysis. For example, the method for determining a hypercoagulable condition may be carried out using the clotting curve determination, as described herein.

Alternately, an angle deviation from a presumed normal coagulation sample may be derived through the use of high standard samples which correspond with the elevated levels of one or more blood components consistent with a hypercoagulable condition. For example, high standard solutions containing elevated levels of fibrinogen and Factor VIII may be used. Fibrinogen activity conversion data may be recorded for the high standard. The angle θ° may be determined using the methods described herein. The angle ascertained for the high reference standards may serve as a reference against which to make hypercoagulability determinations for blood samples of individuals (i.e., those being tested) by comparing the angles. An angle θ° correspondence with a high standard angle (θ°_{HS}) which is derived from the clotting data for a test run with the sample of a person may be used as a positive indicator of a hypercoagulable condition for that person. Correspondence and deviations from a test sample specimen data, in particular the angle, with the high standard data, may be circumscribed to assign a sample (and essentially the individual of that sample) within or without a classification or category of a prothrombotic disorder, and, in particular, a hypercoagulable condition.

Examples are set forth wherein high standard solutions were prepared and compared with samples of persons having presumed normal coagulation. According to the following examples, the presence of hypercoagulable conditions was determined for patient samples. Samples were prepared as follows. High standard samples were prepared which contained elevated levels of fibrinogen and Factor VIII. Cryoprecipitate was used in conjunction with the sample preparation. Cryoprecipitate is a blood product prepared from plasma and contains concentrations of proteins, including von Willebrand factor, fibrinogen, factor VIII and fibronectin. Cryoprecipitate may be obtained, for example, by a slow thawing of fresh frozen plasma at a low temperature, such as at about 4° C., and centrifuging at a low temperature to precipitate the aforementioned proteins, including fibrinogen and Factor VIII. Cryoprecipitate may be quantified in units, with each unit being defined as that amount or portion obtained from 250 ml plasma (which essentially is the amount of one single fresh frozen plasma (or FFP)). One unit of cryoprecipitate (CPP) contains about at least 80 IU (international units) Factor VIII and about 250 mg of fibrinogen. According to some measurements, for example, each 15 ml unit of cryoprecipitate may contain about 100 IU of factor VIII and about 350 mg of fibrinogen, von Willebrand factor, factor XIII, and fibronectin.

The cryoprecipitate is concentrated from the original plasma volume to a volume of about 10 to 15 ml. The cryoprecipitate may be stored, preferably at a temperature of about 0 C. After storage, the cryoprecipitate is reconstituted. In this example, about 10 ml of a saline solution was used to make up the cryoprecipitate concentrate to 25 ml total volume. The concentration of Factor VIII and of fibrinogen in the 25 ml sample was:

80 IU/25 ml=3.2 IU Factor VIII/ml CPP

250 mg/25 ml=10 mg FBG/ml CPP

A high standard solution was prepared using normal plasma and CPP. The high standard contained Factor VIII in an amount greater than the plasma of a person with presumed normal coagulation (that is neither hypocoagulable nor hypercoagulable). Normal plasma has about 1 IU Factor VIII per 1 ml. A high standard (HS) was prepared based on mixing CPP in a 1:1 ratio by volume with normal plasma, which results in the following:

(3.2 IU Factor VIII+1.0 IU Factor VIII)/2=2.1 IU Factor VIII/ml in the high standard.

The high standard (HS) contained an amount of Factor VIII which was about 210% greater than the Factor VIII content in plasma of a person with presumed normal coagulation. The high standard (HS) also contained an amount of fibrinogen which was greater than the fibrinogen of the plasma of a person with presumed normal coagulation. The fibrinogen content of the high standard was calculated as follows:

The CPP, as considered above, contains about 10 mg/ml fibrinogen (or 1000 mg/di). Plasma of a person with presumed normal coagulation contains about 300 mg/dl of fibrinogen. Considering the fibrinogen content in the high standard:

((1000+300) mg/dl)/2=650 mg/dl FBG in the high standard (HS) or 650/300=2.17 or, expressed in other terms, 217% greater than the FBG content of presumed normal plasma.

The high standard prepared contained about 200% greater levels of Factor VIII found in the plasma of persons with presumed normal coagulation. Factor VIII is associated with a shortened prothrombin time (PT), which is the period of time calculated from the addition of a reagent used to activate the clotting process (e.g., thromboplastin-calcium) to a point where the conversion of fibrinogen to fibrin begins (i.e., the formation of the first clot).

In accordance with the clotting curves illustrated in FIGS. 2-5, the PT is shown, and is represented by T1. The clotting curve in FIG. 7 illustrates a representative curve for a blood sample of a person with a hypercoagulable condition (illustrated for example as a high standard reference). The PT of a person with a hypercoagulable condition generally is shorter than a PT for example of a person presumed to have normal coagulability (that is, for this example, a person who has neither a hypocoagulable nor a hypercoagulable condition). However, this does not have to always be the case, and therefore, mere consideration of the PT with respect to high standard comparison or analysis is not determinative of the presence of a hypercoagulable condition. The analysis was conducted to include determining an angle for the slope of the clotting curve as a normal line representing time t=T2S, and paralleling the y-axis, and the angle determined by the line taken from T2S and extending to XT (which in other words is the time to maximum acceleration (T2 in FIG. 6)). FIG. 6 illustrates an example of the clotting curve and the line (L) whose slope was used to determine the indicating angle θ°.

The testing of samples was carried out, and time and absorbance data, including the PT and XT times, were recorded. The test included twenty-two samples. High standards were prepared to contain an amount of a clotting factor at a level which is greater than that contained in the blood or blood component of a person considered to have a clinically normal coagulation. According to the example, two high standards containing the higher amounts of a clotting factor (here containing higher amounts of Factor VIII) were also analyzed. The twenty-two samples were run with three different clotting agents, and two high standard samples were prepared and run for each of the three clotting agents. The data from the analysis is presented in Table 12. The twenty-two samples were from the blood of persons presumed to have normal coagulation. The time to maximum acceleration (XT), the point at which the angle θ° is completed, ranged, for the samples evaluated, from 12.2 seconds to 14.6 seconds (for the Dade TPC coagulation agent). The information utilized to determine an indicator for hypercoagulable condition may be obtained within about 14 seconds. Accordingly, a determination of hypercoagulability, may be completed within about thirty seconds. In accordance with the evaluation, two high standard samples, HSx1 and HSx2 were included for each clotting agent. HSx1 and HSx2 represent high standard samples and are included on the results in Table 12 as respective references, HSTPC1 and HSTPC2 (for the Dade TPC clotting agent).

Analyses were conducted using three different clotting agents. One was TPC (Dade thromboplastin C Plus, which is a thromboplastin with calcium). Each of the twenty-two samples also was run with this clotting agent added (see the results identified on Table 12 as “Tp 1”). Another clotting agent was used, namely, BTP, or bovine thrombin, which is obtained from bovine plasma and is a clotting enzyme that facilitates the formation of fibrin clots from fibrinogen. BTP is a serine protease and functions by cleaving Arginine-Glycine bonds in fibrinogen. Fibrin and fibrinpeptide A and B result from the cleaving. Each of the twenty-two samples was run using the BTP clotting agent (identified on Table 12 as “Tp 2”). A third clotting agent, Innovin, also was used (see Table 12, “Tp 3”).

The angle obtained for the samples in the analysis was determined by using the slope data to obtain a value corresponding to a trigonometric function. According to a preferred embodiment, the measurements were used to correspond with the tangent of the angle. The tangent was determined using the clot slope data obtained from the clotting analysis. The following expressions were used in conjunction with a tangent determination.

tc=XT−T2S (14)

Tm=TEOT−T2S (15)

tan θ=Tm/IUT (16)

In accordance with the expressions (14) (15) and (16), tc/IUX and Tm/IUT are equivalent (see FIG. 6). XT is identified as T2 in FIG. 6.

An apparatus according to the invention may be constructed to include spectrophotometric means for spectroscopically analyzing a sample. For example, a spectrophotometer as described herein may be used to record changes in the absorbance values for fibrinogen during the sample analysis. The clotting curves illustrated in FIGS. 2-8 correspond with fibrinogen transformation. The apparatus may record the values of absorbance in units (which according to preferred embodiments may be instrument units) for each time interval or frequency. The recording frequency may, for example, involve taking the absorbance value of the sample every 1/100^{th} of a second. Other frequencies may be used (e.g., 1/10^{th} of a second). The apparatus may include or be linked with a processor, such as a computer, for handling the data. The data may be stored and processed according to instructions. The instructions may be provided through software programs which are configured to process the data by comparing the data to thresholds of angle deviations from angles obtained for presumed normal coagulation samples or correlations with angles corresponding to high standard data, or ranges corresponding thereto.

According to the method, angle indicator values were determined for a sampling of individuals. Samples were obtained from individuals and run with three different clotting agents, including Dade Thromboplastin C, Dade Innovin and Biopool TP. Each sample was placed into a cuvette and placed into a spectrophotometer. Readings were taken of absorbance values throughout the test. The clotting agent was added to the sample contents as the absorbance values were being recorded. A wavelength of about 660 nm was used for the absorbance analysis. The data was collected and stored for each sample. Twenty-two samples were run and are represented in the analysis. A computer was programmed to manipulate the data to determine angles for each corresponding sample. According to the plot on FIG. 6, the base line T0 to T1 represents 100% of the light on the detector cell after the injection of the clotting agent. As fibrin forms from the fibrinogen in the sample, less light reaches the detector and hence reduces the cell's voltage output. The method was carried out using a configuration in spectrophotometer (i.e., POTENS) which contained instructions to subtract the output from a constant reference value and record the result every 100^{th} of a second. According to preferred embodiments, the spectrophotometer employed has a linear-based photo-optical configuration.

Referring to FIGS. 6-8, angle θ corresponds with the sample's thrombin activity as well as characteristics of the thromboplastin. Optical density on the y axis was recorded in instrument units (IU), and time is on the x axis. The instrument unit ratio (ftr) is subtracted from “2” and is used as the exponent to the “XR”. Each exponent is generated by each individual sample (or specimen) and this value varies depending on the specific sample being tested (and not on the thromboplastin-instrument combination as in the case with the INR).

The data for the twenty-two individual samples and the high standards is reported in Table 12.

TABLE 12

| | | | | | | | | | | |
|---|---|---|---|---|---|---|---|---|---|---|
| | | | | | | | | | | Angle |
| Tp | ID | Pt | Xt | Fg | INR | INRz | Iux | tc | Tc/Iux | θ° |

Dade Thromboplastin C

| | | | | | | | | | | |
|---|---|---|---|---|---|---|---|---|---|---|
| 1 | HSTPC1 | 9.2 | 13.7 | 707 | 0.8 | 1.0 | 27 | 130 | 4.82 | 71.44 |
| 1 | HSTPC2 | 9.0 | 13.4 | 707 | 0.7 | 1.0 | 27 | 131 | 4.85 | 71.52 |
| 1 | 1 | 10.3 | 13.3 | 227 | 0.9 | 1.0 | 6 | 69 | 11.50 | 77.61 |
| 1 | 2 | 9.2 | 12.4 | 202 | 0.8 | 0.9 | 6 | 87 | 14.50 | 78.55 |
| 1 | 3 | 9.4 | 12.5 | 193 | 0.8 | 0.9 | 6 | 91 | 15.17 | 78.70 |
| 1 | 4 | 9.5 | 12.2 | 198 | 0.8 | 0.8 | 5 | 61 | 12.20 | 77.87 |
| 1 | 5 | 10.7 | 14.1 | 143 | 1.0 | 1.1 | 5 | 125 | 25.00 | 80.06 |
| 1 | 6 | 9.5 | 13.2 | 210 | 0.8 | 0.9 | 9 | 129 | 14.33 | 78.50 |
| 1 | 7 | 9.0 | 12.5 | 181 | 0.8 | 0.9 | 7 | 114 | 16.29 | 78.94 |
| 1 | 8 | 10.1 | 13.7 | 227 | 0.9 | 1.0 | 8 | 108 | 13.50 | 78.28 |
| 1 | 9 | 9.9 | 13.2 | 219 | 0.9 | 0.9 | 9 | 112 | 12.44 | 77.95 |
| 1 | 10 | 10.0 | 13.3 | 185 | 0.9 | 1.0 | 7 | 113 | 16.14 | 78.91 |
| 1 | 11 | 9.9 | 13.2 | 206 | 0.9 | 1.0 | 7 | 93 | 13.29 | 78.22 |
| 1 | 12 | 10.5 | 13.7 | 185 | 1.0 | 1.0 | 6 | 99 | 16.50 | 78.98 |
| 1 | 13 | 9.9 | 13.8 | 202 | 0.9 | 1.0 | 8 | 125 | 15.63 | 78.80 |
| 1 | 14 | 9.4 | 12.5 | 214 | 0.8 | 0.9 | 7 | 100 | 14.29 | 78.49 |
| 1 | 15 | 10.7 | 13.9 | 160 | 1.0 | 1.0 | 6 | 108 | 18.00 | 79.24 |
| 1 | 16 | 11.4 | 14.6 | 147 | 1.1 | 1.1 | 5 | 93 | 18.60 | 79.34 |
| 1 | 17 | 9.9 | 13.8 | 227 | 0.9 | 1.0 | 10 | 125 | 12.50 | 77.97 |
| 1 | 18 | 9.8 | 13.2 | 252 | 0.9 | 1.0 | 10 | 111 | 11.10 | 77.45 |
| 1 | 19 | 8.8 | 12.7 | 273 | 0.7 | 0.9 | 10 | 108 | 10.80 | 77.32 |
| 1 | 20 | 9.4 | 12.6 | 181 | 0.8 | 0.9 | 6 | 99 | 16.50 | 78.98 |
| 1 | 21 | 10.8 | 13.5 | 172 | 1.0 | 1.0 | 4 | 71 | 17.75 | 79.20 |
| 1 | 22 | 9.9 | 13.2 | 177 | 0.9 | 1.0 | 6 | 113 | 18.83 | 79.37 |
| average | | 9.9 | 13.2 | | | | | | | |

Dade Innovin

| | | | | | | | | | | |
|---|---|---|---|---|---|---|---|---|---|---|
| 2 | HSINN1 | 7.9 | 11.4 | 707 | 0.8 | 0.9 | 13 | 97 | 7.46 | 75.18 |
| 2 | HSINN2 | 7.7 | 11.3 | 707 | 0.8 | 0.9 | 12 | 100 | 8.33 | 75.90 |
| 2 | 1 | 8.4 | 11.9 | 178 | 0.8 | 1.0 | 5 | 123 | 24.60 | 80.02 |
| 2 | 2 | 7.6 | 10.6 | 160 | 0.7 | 0.8 | 4 | 144 | 36.00 | 80.69 |
| 2 | 3 | 8.6 | 11.5 | 151 | 0.8 | 1.0 | 4 | 156 | 39.00 | 80.81 |
| 2 | 4 | 8.3 | 11.2 | 132 | 0.8 | 0.9 | 3 | 106 | 35.33 | 80.67 |
| 2 | 5 | 8.9 | 12.1 | 105 | 0.9 | 1.0 | 2 | 104 | 52.00 | 81.14 |
| 2 | 6 | 8.2 | 11.0 | 151 | 0.8 | 0.9 | 3 | 65 | 21.67 | 79.73 |
| 2 | 8 | 8.1 | 11.5 | 178 | 0.8 | 1.0 | 4 | 122 | 30.50 | 80.43 |
| 2 | 10 | 9.1 | 11.9 | 160 | 0.9 | 1.0 | 3 | 123 | 41.00 | 80.87 |
| 2 | 11 | 8.4 | 11.2 | 169 | 0.8 | 0.9 | 3 | 103 | 34.33 | 80.62 |
| 2 | 12 | 9.4 | 12.3 | 160 | 0.9 | 1.0 | 4 | 143 | 35.75 | 80.68 |
| 2 | 13 | 8.4 | 11.6 | 169 | 0.8 | 1.0 | 5 | 135 | 27.00 | 80.21 |
| 2 | 14 | 8.2 | 11.0 | 169 | 0.8 | 0.9 | 4 | 140 | 35.00 | 80.65 |
| 2 | 16 | 10.5 | 13.6 | 105 | 1.0 | 1.2 | 3 | 171 | 57.00 | 81.23 |
| 2 | 17 | 8.4 | 11.1 | 187 | 0.8 | 0.9 | 4 | 99 | 24.75 | 80.03 |
| 2 | 19 | 7.9 | 10.9 | 196 | 0.8 | 0.9 | 5 | 148 | 29.60 | 80.38 |
| 2 | 20 | 7.8 | 10.5 | 141 | 0.8 | 0.8 | 3 | 122 | 40.67 | 80.86 |
| 2 | 21 | 8.9 | 12.4 | 141 | 0.9 | 1.1 | 4 | 148 | 37.00 | 80.73 |
| 2 | 22 | 8.5 | 11.4 | 141 | 0.8 | 0.9 | 4 | 146 | 36.50 | 80.71 |
| Average | | 8.5 | 11.5 | | | | | | | |

BioPool

| | | | | | | | | | | |
|---|---|---|---|---|---|---|---|---|---|---|
| 3 | HSBPT1 | 10.9 | 14.9 | 707 | 1.1 | 1.5 | 19 | 112 | 5.90 | 73.36 |
| 3 | HSBPT2 | 11.2 | 15.3 | 707 | 1.1 | 1.6 | 19 | 123 | 6.47 | 74.13 |
| 3 | 1 | 11.3 | 14.6 | 212 | 1.1 | 1.4 | 7 | 118 | 16.86 | 79.05 |
| 3 | 2 | 9.9 | 13.7 | 188 | 0.9 | 1.2 | 7 | 150 | 21.43 | 79.71 |
| 3 | 3 | 10.8 | 14.0 | 154 | 1.0 | 1.3 | 5 | 144 | 28.80 | 80.33 |
| 3 | 4 | 11.2 | 14.4 | 158 | 1.1 | 1.3 | 6 | 131 | 21.83 | 79.75 |
| 3 | 5 | 12.2 | 15.4 | 129 | 1.3 | 1.5 | 5 | 148 | 29.60 | 80.38 |
| 3 | 6 | 11.0 | 14.3 | 188 | 1.1 | 1.3 | 6 | 132 | 22.00 | 79.77 |
| 3 | 7 | 10.5 | 13.4 | 178 | 1.0 | 1.2 | 4 | 84 | 21.00 | 79.66 |
| 3 | 8 | 11.1 | 14.8 | 207 | 1.1 | 1.4 | 7 | 120 | 17.14 | 79.10 |
| 3 | 9 | 11.1 | 14.6 | 183 | 1.1 | 1.4 | 7 | 136 | 19.43 | 79.46 |
| 3 | 10 | 11.3 | 14.7 | 168 | 1.1 | 1.4 | 7 | 150 | 21.43 | 79.71 |
| 3 | 11 | 10.6 | 14.3 | 192 | 1.0 | 1.3 | 6 | 131 | 21.83 | 79.75 |
| 3 | 12 | 11.5 | 14.6 | 158 | 1.2 | 1.4 | 4 | 103 | 25.75 | 80.12 |
| 3 | 13 | 10.8 | 14.5 | 188 | 1.0 | 1.4 | 6 | 105 | 17.50 | 79.16 |
| 3 | 14 | 10.5 | 13.7 | 183 | 1.0 | 1.3 | 4 | 77 | 19.25 | 79.43 |
| 3 | 15 | 11.3 | 14.6 | 149 | 1.1 | 1.4 | 5 | 137 | 27.40 | 80.24 |
| 3 | 16 | 12.6 | 15.9 | 129 | 1.4 | 1.6 | 4 | 140 | 35.00 | 80.65 |
| 3 | 17 | 10.9 | 14.3 | 217 | 1.1 | 1.3 | 6 | 87 | 14.50 | 78.55 |
| 3 | 18 | 10.8 | 14.0 | 222 | 1.0 | 1.3 | 6 | 94 | 15.67 | 78.81 |
| 3 | 19 | 10.2 | 14.0 | 241 | 0.9 | 1.3 | 9 | 134 | 14.89 | 78.64 |
| 3 | 20 | 10.1 | 13.5 | 173 | 0.9 | 1.2 | 5 | 96 | 19.20 | 79.42 |
| 3 | 21 | 11.2 | 14.6 | 163 | 1.1 | 1.4 | 5 | 122 | 24.40 | 80.00 |
| 3 | 22 | 11.2 | 14.2 | 168 | 1.1 | 1.3 | 4 | 82 | 20.50 | 79.60 |
| average | | 11.0 | 14.4 | | | | | | | |

Table 12 lists two high standard values HSx1 and HSx2 for each clotting agent used. A total of six high standard values are reported in Table 12 for three different clotting agents. The high standard values are listed as HSx1 and HSx2, where x corresponds to the clotting agent, e.g., x=TPC for Dade Thromboplastin C Plus, x=INN for Dade Innovin, and x=BPT for BioPool Thromboplastin. Each of the high standards (HSx) shows significantly smaller (or more acute) angles θ when compared with the angles (θ) for the presumed normal coagulation condition patient samples. Turning to the sample run for the first clotting agent Dade thromboplastin C Plus, the smallest angle θ of the twenty two samples run, was sample ID 19, whose corresponding angle θ° was 77.32°. Considering the high standards (HSTPC1 and HSTPC2), the deviation was about 8% (percent) of the individual sample having the smallest angle θ (which is sample ID 19) and the high standard having the largest angle (HSTPC2). Each of the other clotting agents was also considered. For the TP 2, which is the Dade Innovin, sample ID 6 produced the smallest angle θ of the samples run. (It is noted that samples 7, 9, 15 and 18 were not obtained for the Tp2—Dade Innovin.) When the TP2 sample having the smallest angle was compared with the high standard having the largest angle θ, there was about a 4.8% (percent) difference. Similarly, there was a difference of about 5.6% (percent)) between the sample having the smallest angle θ (which is sample 17 for the Tp 3—Biopool TPC) and the high reference sample (HSBPT2) (which was the high standard having the largest angle). The data in Table 12 illustrates a relationship between the angle and the blood or blood components of an individual. Angles approximating the high standards (HSx) may be considered to be indicative of a hypercoagulable condition. Average values are also included in Table 12 for the PT and XT values for the individual patients (not inclusive of the high standards). According to preferred embodiments, the angle indicators, angle θ, which are determined for each sample, serve as a means for comparison of that sample to a reference standard, such as, for example the high standards listed in Table 12. For example, where an angle determined for a sample is within a particular proximity to an angle corresponding with a high standard, that may be used to assign the sample in a hypercoagulation condition category. For example, the proximity for the angle deviation may be within 3% of the reference, within 5% of the reference, or another number. Preferably, the coagulation agent used for the sample coagulation analysis is compared with a high standard derived for the same clotting agent.

There also may be provided a normal coagulation standard, where individuals having presumed normal coagulation are sampled and their angle values compared as a reference. Conversely, reference angles for samples of individuals known to have a hypercoagulable condition may be determined and used as a reference against which to compare angles derived from the testing of samples from other individuals.

The angle determinations discussed herein in their broadest sense provide a relationship of a clotting condition. More particularly, the relationship is one which may be determinative of a prothrombotic abnormality, such as, for example, a hypercoagulable condition. The present method and apparatus enable the use of clotting agents with a blood sample or blood component sample to derive a indicator of a hypercoagulable condition.

While the invention has been described with reference to specific embodiments, the description is illustrative and is not to be construed as limiting the scope of the invention. The sample container used to contain the sample may comprise a vial, or cuvette, including, for example, the sample container disclosed in our U.S. Pat. No. 6,706,536. For example, although described in connection with body fluids of a human, the present invention has applicability to veterinary procedures, as well, where fluids are to be measured or analyzed. Various modifications and changes may occur to those skilled in the art without departing from the spirit and scope of the invention described herein and as defined by the appended claims.

What is claimed is:

1. A method for determining a prothrombotic condition in a living being comprising:
determining an angle value for a reference standard in a coagulation study of at least one sample having presumed normal coagulation or at least one standard having coagulation which is considered to have coagulation which is not normal;
assigning the angle value of said at least one reference standard as a reference value;
obtaining an angle value for the sample of an individual;
comparing the angle value of the individual sample with said reference value;
assigning a status based on the results of the comparison.

2. The method of claim 1, wherein assigning a value comprises obtaining time and absorbance values for a sample undergoing clotting activity, determining from said time and absorbance values a slope, and obtaining from said data an indicator used to signify the presence of a prothrombotic condition.

3. The method of claim 1, wherein the prothrombotic condition comprises a hypercoagulable condition.

4. The method of claim 1, wherein said indicator comprises an angle defined at least in part by said slope representing time and absorbance values.

5. The method of claim 1, wherein said angle is defined by said slope and a line taken at the time approximating the start of the acceleration of fibrinogen conversion in a coagulation reaction.

6. The method of claim 5, wherein the time approximating the start of the acceleration of fibrinogen conversion in a coagulation reaction is a time T2S.

7. The method of claim 6, wherein the angle is formed at the intersection of t=T2S and the slope.

8. The method of claim 1, wherein the angle is defined by the slope and at least one line wherein c=x, where c represents absorbance value plotted against time, and wherein x is less than the concentration represented by the absorbance value at cT2S, and greater than c=0.

9. The method of claim 1, wherein the reference standard is based on at least one sample having presumed normal coagulation.

10. The method of claim 1, wherein the reference sample is based on at least one sample having an increased amount of at least one clotting component.

11. The method of claim 10, wherein the at least one clotting component is Factor VIII and the sample has an increased level of Factor VIII, relative to the Factor VIII content of a sample of a person with presumed normal coagulation.

12. The method of claim 1, wherein a plurality of samples from individuals having presumed normal coagulation are used to obtain a standard reference angle.

13. The method of claim 10, wherein a high standard sample containing an increased amount or at least one clotting component is used to obtain a high standard reference angle.

14. The method of claim 1, wherein the status is the presence of a hypercoagulable condition.

15. A method for determining a prothrombotic condition in a living being comprising:
conducting a clotting reaction for a sample of blood or blood components by adding a reagent to the blood or blood component,
recording values for time and absorbance during the clotting reaction;
determining an indicator for a prothrombotic condition based on a trigonometric function using the time and absorbance values for the sample.

16. A method for determining a hypercoagulable condition in a human comprising:
a. determining a slope value of a zero order kinetic line representing the reaction rate of the transformation of fibrinogen in a blood sample to fibrin, by reacting a blood sample of a human with a coagulant and monitoring optical density changes associated with the fibrinogen transformation;
b. comparing the slope value for the said zero order kinetic line with a predetermined range of slope values which correspond with a state of hypercoagulability.

17. The method of claim 16, wherein said determination is carried out within a duration of no longer than about 45 seconds.

18. The method of claim 16, wherein the coagulant reacted with the blood sample is thromboplastin.

19. The method of claim 17, wherein the coagulant reacted with the blood sample is thromboplastin.

20. The method of claim 16, wherein the coagulant reacted with the blood sample is innovin.

21. The method of claim 20, wherein said determination is carried out within a duration of no longer than about 30 seconds.

22. The method of claim 16, wherein said zero order kinetic line representing the reaction rate of the transformation of fibrinogen in a blood sample to fibrin is derived by determining, upon the addition of a coagulant to a blood sample containing fibrin, a concentration value cT2S corresponding with a time to start (T₂S) of the simulated zero order kinetic to the concentration value cT2 corresponding with a last highest absorbance value (T₂).

23. The method of claim 16, wherein said slope value of a zero order kinetic line representing the fibrinogen transformation for a blood sample is derived by monitoring with a spectrophotometer the percent transmittance of light passing through the sample over the time during which fibrinogen in the sample is being transformed to fibrin.

24. The method of claim 23, wherein said slope value corresponds with a tangent of a maximum acceleration region of the plot of the time value of the reaction against a value based on the percent transmittance.

25. The method of claim 1, wherein the deviation of an angle value obtained for a sample of an individual is considered to correspond with a prothrombotic condition where the comparison results in a percentage deviation of about 5% or greater from an angle obtained from a sample of an individual considered to have normal coagulation.

26. The method of claim 16, wherein the slope value that corresponds with a state of hypercoagulability is at least two or more standard deviations from a reference slope value.

27. The method of claim 16, wherein the slope value corresponding with a state of hypercoagulability is equal to or greater than about three standard deviations from the value of a reference standard angle value.

28. A method for determining a hypercoagulable condition in a human comprising:
a. developing a series of analog electrical voltage signals having voltage amplitudes, proportional to an optical density of a liquid sample containing fibrinogen;
b. converting the developed analog voltage signals into a series of digital voltage value signals;
c. adding a coagulant into the liquid sample, thereby producing an abrupt change in the optical density of the liquid sample, said abrupt change producing an abrupt change in the amplitude of the electrical analog signals which, in turn, produces an abrupt change in the value of said digital voltage signals, the value of said digital voltage signals being directly indicative of fibrinogen concentration in the liquid sample;
d. recording an instant time T₀ of said abrupt change in said value of said digital voltage signal;
e. monitoring said voltage digital signal values for coagulant activity;
f. recording an instant time T₁ corresponding to the start of clot formation;
g. monitoring said voltage digital signal values for further fibrinogen concentration quantities;
h. recording an instant time T₂S which corresponds to a starting point of a simulated zero order kinetic and recording the value of the voltage digital signal of a fibrinogen concentration C_{T2S};
i. recording an instant time T₂ and the value of the voltage digital signal of a predetermined fibrinogen concentration quantity C_{T2}, wherein T₂ corresponds with the point where the maximum acceleration of the conversion of fibrinogen to fibrin occurs;
j. recording an elapsed time between T₀ and T₂ which defines a time to maximum acceleration of the conversion of fibrinogen to fibrin (TX) from coagulant injection in step (c);
k. monitoring for a differential change in the voltage digital signal values that include said predetermined fibrinogen concentration quantity C_{T2};
l. wherein said fibrinogen concentration quantity C_{T2} and said time T₂ define a maximum acceleration point (MAP) and a time to maximum acceleration of the conversion of fibrinogen to fibrin from coagulant injection (TX), wherein TX is measured as the elapsed time from the time of the coagulant injection T₀ to the time to maximum acceleration T₂;
m. monitoring voltage digital signal values at times T₂S and T₂ for respective predetermined fibrinogen concentration quantities C_{T2S} and C_{T2}, with the difference between quantities C_{T2S} and C_{T2} being a first differential IUX, and with the difference between times T2S and T2 being a second differential tc;
n. comparing a value based on IUX/tc with a predetermined range, of values which correspond with a state of hypercoagulability.

29. The method of claim 28, wherein comparing said zero order fibrinogen transformation rate with a predetermined range of slope values which correspond with a state of hypercoagulability includes determining an indicator angle based on a tangent derived from the expression IUX/tc, and wherein the predetermined range of values correspond with angle values.

30. The method of claim 1, wherein the method is carried out within about thirty seconds.

31. The method of claim 28, wherein the deviation of a slope value obtained for a sample of an individual is considered to correspond with a prothrombotic condition where the comparison results in a percentage deviation of about 5% or greater from a slope value obtained from a sample of an individual considered to have normal coagulation.

32. The method of claim 28, wherein the slope value that corresponds with a state of hypercoagulability is at least two or more standard deviations from a reference slope value.

33. The method of claim 28, wherein the slope value corresponding with a state of hypercoagulability is equal to or greater than about three standard deviations from the value of a reference standard angle value.

34. An apparatus for determining a prothrombotic condition, said apparatus having a processor, and a computer chip preprogrammed with a set of instructions for cooperating with the output of a photodetection device which provides electrical data to said processor as a function of the optical density for a sample being analyzed, said apparatus having input means and storage means for storing data, said set of instructions including instructions for determining the presence of a hypercoagulable condition based on the steps set forth in claim 1.

35. The method of claim 1, further comprising an article for determining the presence of a hypercoagulable condition, the article including storage media with stored instructions which can be read and processed with a processor to determine whether a slope value corresponds to a value indicative of a hypercoagulable condition.

36. The method of claim 35, wherein the slope value comprises an angle derived from a tangent of the clot slope curve.
